(19) United States
(12) Patent Application Publication
(10) Pub. No.: US 2024/0366729 A1
(43) Pub. Date: Nov. 7, 2024

(54) COMBINATION OF A NURR1 AGONIST WITH AT LEAST ONE OF AN ALDOSTERONE ANTAGONIST, AN INSULIN MODULATOR AND A SULFONYLUREA

(71) Applicant: GENESIS PHARMA SA, Athens (GR)

(72) Inventor: George VOGIATZIS, Athens (GR)

(21) Appl. No.: 18/684,454

(22) PCT Filed: Aug. 26, 2022
(86) PCT No.: PCT/IB2022/058008

§ 371 Date: Feb. 16, 2024

(30) Foreign Application Priority Data
Aug. 27, 2021 (GR) 20210100572

(51) Int. Cl. A61K 38/26; A61K 31/4706; A61K 31/569; A61K 31/64; A61K 45/06; A61P 9/10; A61P 25/28

(52) U.S. Cl. CPC A61K 38/26; A61K 31/4706; A61K 31/569; A61K 31/64; A61K 45/06; A61P 9/10; A61P 25/28

(57) ABSTRACT

The present invention provides a combination comprising: (a) a first component which is a Nurr1 agonist; and (b) at least one additional component selected from: (i) an aldosterone antagonist; (ii) an insulin modulator; and (iii) a sulfonylurea. Said combinations are suitable for the treatment of stroke and other neurodegenerative and neuroinflammatory disorders and for treating and/or preventing ischemia and/or reperfusion injury in various vital organs, including the brain and the heart. Further aspects of the invention relate to pharmaceutical products and pharmaceutical compositions comprising said combinations according to the invention, and methods of treatment using the same.

### FIELD OF INVENTION

The present invention provides a combination suitable for the treatment of one or more of stroke, a neurodegenerative disorder, neuroinflammation, a neuroinflammatory disorder, and for treating and/or preventing ischemia and/or reperfusion injury in various vital organs, including the brain and the heart. More specifically, the combinations of the invention comprise a Nurr1 agonist.

Further aspects of the invention relate to pharmaceutical products and pharmaceutical compositions comprising said combinations, and methods of treatment using the same.

### BACKGROUND

Stroke is a prominent cause of serious, long-term disability and the third leading cause of death in the United States. Stroke is caused by lack of blood flow in the brain (ischemic stroke) or by bleeding in the brain (haemorrhagic stroke) and both conditions result in brain cell death. Ischemic strokes comprise over 88% of all strokes, making them the most common type of cerebrovascular injury. Ischemic conditions in the brain cause neuronal death, leading to permanent sensorimotor deficits. As well as leading to significant physical disabilities, stroke is also associated with memory loss and depression.

Stroke is the second most important cause of death globally, accounting for about 6 million deaths in 2016 according to the World Health Organisation. The high burden of stroke worldwide suggests that primary prevention strategies are either not widely implemented or not sufficiently effective. Guidelines are available for the management of acute ischemic stroke (Powers W J, et al. Stroke. 2019; 50: e344-e418). Interestingly, the guidelines conclude that at present, no pharmacological or non-pharmacological treatments with putative neuroprotective actions have demonstrated efficacy in improving outcomes after ischemic stroke, and therefore, other neuroprotective agents are not recommended. Guidelines also exist for the management of haemorrhagic stroke; however, they do not recommend therapies for the management of the neurodegenerative consequences of haemorrhagic stroke other than rehabilitation (Hemphill J C 3rd, et al. Stroke. 2015; 46: 2032-2060).

The data above clearly indicate that presently effective pharmacologic treatments of ischemic or haemorrhagic stroke are lacking and that there is a need for treatments that are neuroprotective in patients with stroke. An effective treatment of the reperfusion injury that is associated with stroke has the potential to offer neuroprotection. However, attempts so far to develop effective neuroprotective treatments for stroke patients, which are based on decreasing the reperfusion injury have not been successful (Savitz S I, et al. Stroke. 2017; 48: 3413-3419; Patel R A G, et al. Prog Cardiovasc Dis. 2017; 59: 542-548). Previous studies in the area of cardioprotection and reperfusion injury have revealed the surprising finding that combination therapies, not indicated for the treatment of cardiovascular disorders, if used at dose levels that are lower than the ones indicated for these other conditions can result in significant synergistic effects that protect against myocardial reperfusion injury (WO 2017/077378; U.S. Pat. No. 10,172,914; Genesis Pharma SA). Earlier studies by the Applicant have shown that combination therapy using a sulfonyl urea and a second active component has potential therapeutic applications in the treatment of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection against neurotoxic drugs (WO 2021/005147; Genesis Pharma SA). In particular, studies by the Applicant demonstrated that a combination of glibenclamide and exenatide and/or potassium canrenoate can reduce the extent of cerebral infarction and/or improve neurological severity score and/or improve motor performance.

Treatments showing neuroprotective effects are also expected to be useful in the treatment of neurodegenerative disorders. Neurodegenerative disorders are due to a progressive loss of structure or function of neurons, which eventually leads to the death of neurons. They include diseases such as Parkinson's disease, Alzheimer's disease, Huntington's disease, amyotrophic lateral sclerosis (ALS) and vascular dementia that are currently incurable. Neuroinflammatory pathways are also understood to play an important role in neurodegenerative diseases (Chen et al 2016; Liddelow et al 2017). Vascular dementia is dementia caused by problems in the supply of blood to the brain, typically a series of minor strokes, leading to worsening cognitive decline that occurs step by step. The term refers to a syndrome consisting of a complex interaction of cerebrovascular disease and risk factors that lead to changes in the brain structures due to strokes and lesions, and resulting changes in cognition. Currently, there are no medications that have been approved specifically for the prevention or treatment of vascular dementia. The currently approved therapies for Alzheimer's disease provide only modest benefits (Atri A. Med Clin North Am. 2019; 103: 263-293) and robust evidence of their efficacy is lacking. A number of pharmacologic treatments are available for managing the motor and non-motor symptoms in Parkinson's disease, but they are essentially symptomatic treatments and eventually induce dyskinesias while none of them provides neuroprotection (Chaudhuri K R, et al. Parkinsonism Relat Disord. 2016; 33 (Suppl 1): S2-S8). There are currently only two approved two drugs (riluzole and edaravone) that slow down the progress of amyotrophic lateral sclerosis, albeit modestly, and there is no approved therapy for Huntington's disease.

There is therefore a clear need for further and better treatments that offer neuroprotection, particularly in the context of treating stroke and neurodegenerative diseases, and/or treatments that can inhibit neuroinflammation.

### STATEMENT OF INVENTION

The present invention provides combinations which are suitable for the prevention or treatment of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for use in providing cardioprotection against cardiotoxic drugs, or for use in providing neuroprotection.

Advantageously, the presently claimed combinations and other aspects of the invention provide a treatment which is more efficacious and provides superior clinical outcomes compared to therapies that employ a single active pharmaceutical agent. In particular, the presence of a Nurr1 agonist brings into play an additional and distinct mechanism of action, which is believed to be particularly relevant to neurodegeneration, as compared to the combinations described in WO 2017/077378 and WO 2021/005147. Moreover, in certain embodiments, the presently claimed combination enables the constituent components to be used in lower dosages than those taught in the literature.

A first aspect of the invention relates to a combination comprising:
(a) a first component which is a Nurr1 agonist; and
(b) at least one additional component selected from:
   (i) an aldosterone antagonist;
   (ii) an insulin modulator; and
   (iii) a sulfonylurea.

A second aspect of the invention relates to a pharmaceutical composition comprising a combination as described above and a pharmaceutically acceptable carrier, diluent or excipient.

A third aspect of the invention relates to a pharmaceutical product comprising:
(a) a first component which is a Nurr1 agonist; and
(b) at least one additional component selected from:
   (i) an aldosterone antagonist;
   (ii) an insulin modulator; and
   (iii) a sulfonylurea

A fourth aspect of the invention relates to a combination or pharmaceutical composition or pharmaceutical product as described above, for use in the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for use in providing cardioprotection against cardiotoxic drugs, or for use in providing neuroprotection.

A fifth aspect of the invention relates to a method of treating and/or preventing one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection, said method comprising simultaneously, sequentially or separately administering to a subject in need thereof:
(a) a first component which is a Nurr1 agonist; and
(b) at least one additional component selected from:
   (i) an aldosterone antagonist;
   (ii) an insulin modulator, and
   (iii) a sulfonylurea.

A sixth aspect of the invention relates to the use of:
(a) a first component which is a Nurr1 agonist; and
(b) at least one additional component selected from:
   (i) an aldosterone antagonist;
   (ii) an insulin modulator, and
   (iii) a sulfonylurea;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

A seventh aspect of the invention relates to the use of a combination comprising:
(a) a first component which is a Nurr1 agonist; and
(b) at least one additional component selected from:
   (i) an aldosterone antagonist;
   (ii) an insulin modulator, and
   (iii) a sulfonylurea;
for treating and/or preventing ischemia and/or reperfusion injury in an ex vivo organ prior to or during transplantation.

### DETAILED DESCRIPTION

The preferred embodiments set out below are applicable to any of the above-mentioned aspects of the invention as appropriate.

As used herein, a structural analogue, also known as a chemical analogue, is a compound having a structure similar to that of another compound, but differing from it in respect to a certain component. It can differ in one or more atoms, functional groups, or substructures, which are replaced with other atoms, groups, or substructures. A structural analogue can be imagined to be formed, at least theoretically, from the other compound. Structural analogues are often isoelectronic.

As used herein, functional analogues are chemical compounds that have similar physical, chemical, biochemical, or pharmacological properties to that of another compound. Functional analogues are not necessarily structural analogues with a similar chemical structure.

### Nurr1 Agonists

The combinations, pharmaceutical compositions and pharmaceutical products described herein comprise a Nurr1 agonist in combination with at least one second active agent.

The Nurr1 receptor, also known as the nuclear receptor 4A2 (NR4A2; nuclear receptor subfamily 4 group A member 2) is a protein that in humans is encoded by the NR4A2 gene. NR4A2 is a member of the nuclear receptor family of intracellular transcription factors and plays a key role in the maintenance of the dopaminergic system of the brain (Sacchetti P, et al, 2006). Mutations in this gene have been associated with disorders related to dopaminergic dysfunction, including Parkinson's disease and schizophrenia. Nurr1 is also understood to play a role in the neuroinflammatory pathways associated with certain neurodegenerative disorders, for example, ALS and Alzheimer's (Valsecchi et al. Dis. Model Mech. 2020; 13(5):dmm043513; Jeon et al. Aging Dis., 2020; 11(3):705-724).

In the CNS, inflammation can result from activated microglia and other pro-inflammatory factors, such as bacterial lipopolysaccharide (LPS). LPS binds to toll-like receptors (TLR), which induces inflammatory gene expression by promoting signal-dependent transcription factors. NR4A2 has been shown to protect dopaminergic neurons from LPS-induced inflammation, by reducing inflammatory gene expression in microglia and astrocytes. When a short hairpin for NR4A2 was expressed in microglia and astrocytes, these cells produced inflammatory mediators, such as TNFα, NO synthase and IL-1p, supporting the conclusion that reduced NR4A2 promotes inflammation and leads to cell death of dopaminergic neurons. NR4A2 interacts with the transcription factor complex NF-κB-p65 on the inflammatory gene promoters. However, NR4A2 is dependent on other factors to be able to participate in these interactions. NR4A2 needs to be sumoylated and its co-regulating factor, glycogen synthase kinase 3, needs to be phosphorylated for these interactions to occur. Sumolyated NR4A2 recruits CoREST, a complex made of several proteins that assembles chromatin-modifying enzymes. The NR4A2/CoREST complex inhibits transcription of inflammatory genes (Saijo K. et al; 2009).

Nurr1 agonists have been shown to improve behavioral deficits in an animal model of Parkinson's disease (Kim et al. 2015). Post mortem studies showed that Nurr1 expression is diminished in both aged and Parkinson's Disease (PD) post mortem brains (Chu Y et al. 2002; Chu Y, et al. 2006). Furthermore, functional mutations/polymorphisms of Nurr1 have been identified in rare cases of familial late-onset forms of PD (Le W D, et al. 2003). In addition, Nurr1 heterozygous null mice behave like an animal model of PD, as they exhibit a significant decrease in both rotarod performance and locomotor activities associated with decreased levels of dopamine (DA) in the striatum and decreased number of A9 DA neurons (Jiang C, et al. 2005). Taken together, these findings strongly suggest that disrupted function/expression of Nurr1 is related to neurodegeneration of DA neurons and its activation may improve the pathogenesis of PD (Glass C K et al. 2010).

Kim et al (ibid) successfully identified Nurr1 agonists sharing an identical chemical scaffold, 4-amino-7-chloroquinoline, suggesting a critical structure-activity relationship. In particular, amodiaquine and chloroquine were found to stimulate the transcriptional function of Nurr1 through physical interaction with its ligand binding domain (LBD).

Remarkably, these compounds were able to enhance the contrasting dual functions of Nurr1 by further increasing transcriptional activation of mDA-specific genes and further enhancing transrepression of neurotoxic proinflammatory gene expression in microglia. Importantly, these compounds significantly improved behavioral deficits in 6-hydroxydopamine lesioned rat model of PD without any detectable signs of dyskinesia-like behavior. These findings offer proof of principle that small molecules targeting the Nurr1 LBD can be used as a mechanism based and neuroprotective strategy for PD. Suitable Nurr1 agonists can be identified using known assays (see, for example, as described in Kim 2015). In one preferred embodiment, the Nurr1 agonist in the combinations of the invention is selected from amodiaquine, chloroquine, hydroxychloroquine and glafenine, and pharmaceutically acceptable salts thereof:

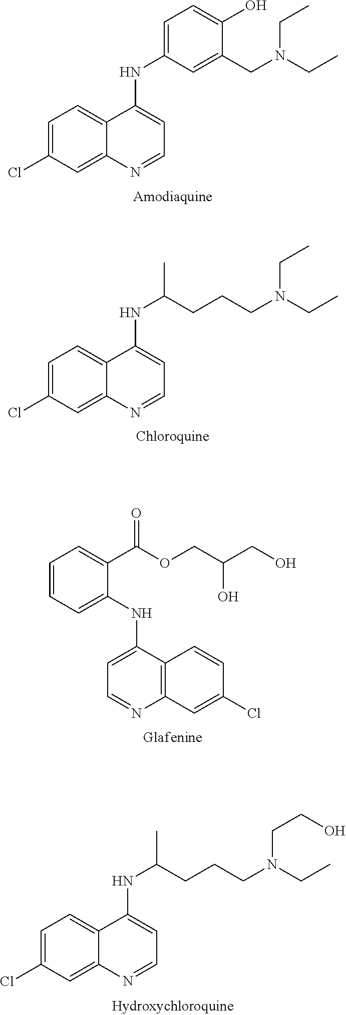 [SMILES: CCN(CC)Cc1cc(Nc2ccnc3cc(Cl)ccc23)ccc1O ; CCN(CC)CCCC(C)Nc1ccnc2cc(Cl)ccc12 ; O=C(OCC(O)CO)c1ccccc1Nc1ccnc2cc(Cl)ccc12 ; CCN(CCO)CCCC(C)Nc1ccnc2cc(Cl)ccc12]

### Amodiaquine

In one particularly preferred embodiment, the Nurr1 agonist in the combination of the invention is amodiaquine, or a pharmaceutically acceptable salt thereof. In one preferred embodiment, the Nurr1 agonist is amodiaquine hydrochloride. More preferably, the Nurr1 agonist is amodiaquine.

Amodiaquine is the compound 4-[(7-chloroquinolin-4-yl)amino]-2[(diethylamino)methyl]-phenol having the structure shown above. Amodiaquine is an alternative first-line drug for uncomplicated malaria that has been shown to induce vasorelaxation in rat superior mesenteric arteries (Oluwatosin et al. 2010). Studies have also shown that amodiaquine attenuates inflammatory events and neurological deficits in a mouse model of intracerebral haemorrhage (ICH) (Kinoshita et al. 2019).

In particular, Kinoshita demonstrated that Nurr1 (NR4A2) was expressed prominently in microglia/macrophages and astrocytes in the perihematomal region in the striatum of mice after ICH. Daily administration of amodiaquine (40 mg/kg, i.p.) from 3 h after ICH induction diminished perihematomal activation of microglia/macrophages and astrocytes. Amodiaquine also suppressed ICH-induced mRNA expression of IL-1β, CCL2 and CXCL2, and ameliorated motor dysfunction of mice, suggesting that Nurr1 serves a novel target for ICH therapy.

Advantageously, the dosages of amodiaquine for use in the present combinations are significantly lower than reported in the literature (e.g. in Kinoshita et al. 2019).

### Sulfonylureas

In certain embodiments, the combinations or pharmaceutical products or pharmaceutical compositions of the invention comprise a sulfonylurea.

Sulfonylureas are a class of oral hypoglycaemic agents that are mainly used in the management of type 2 diabetes and certain forms of monogenic diabetes. They reduce blood glucose levels by stimulating insulin secretion from pancreatic β-cells. Their primary target is the sulfonylurea receptor (SUR1) subunit of the ATP-sensitive potassium (K_{ATP}) channel in the β-cell plasma membrane (Proks P, et al. Diabetes. 2002; 51 (Suppl 3): S368-76; Gribble F M and Reimann F. Diabetologia. 2003; 46: 875-891).

Sulfonylureas are traditionally classified into two generations, consistent with the time of their introduction in the clinic, with differences mainly in their disposition, which allows for a less frequent administration of the drugs that belong to the second generation (Sola D, et al. Arch Med Sci. 2015; 11: 840-8):
the first generation includes chlorpropamide, tolbutamide, acetohexamide, carbutamide, glycyclamide, tolhexamide, metahexamide, and tolazamide; however, these are no longer used in clinical practice;
the second generation includes glibenclamide (glyburide), glibornuride, gliclazide, glipizide, glimepiride, gliquidone, glisoxepide and glyclopyramide. Modified/extended release formulations exist for some of the second-generation sulfonylureas (gliclazide, glipizide).

Preferably, the sulfonylurea in the combination of the invention is a second generation sulfonyl urea. Current guidelines recommend the use of second generation sulfonylureas as second-line therapy in combination with metformin, when inadequate control was achieved with metformin alone, and second generation sulfonylureas may also be used in a three-drug combination treatment if no adequate glycemic control has been achieved with a two-drug combination (Garber A J, et al. Endocr Pract. 2019; 25: 69-100; Inzucchi S E, et al. Diabetes Care. 2015; 38: 140-9). The decision to use a sulfonylurea should take into account patient characteristics and potential adverse events that have been associated with sulfonylureas (Cordiner R L M, Pearson E R. Diabetes Obes Metab. 2019; 21: 761-771).

In one particularly preferred embodiment, the sulfonylurea is a Sur-1 receptor antagonist. Suitable Sur-1 receptor antagonists can be identified using known assays.

In one particularly preferred embodiment, the sulfonylurea is a SUR1-TRPM4 channel antagonist. Suitable SUR1-TRPM4 channel antagonists can be identified using known assays.

The invention also encompasses structural or functional analogues of the sulfonylureas, particularly those that are modified so as to extend the half-life of the agent, for example, conjugates of sulfonylureas.

In one preferred embodiment, the sulfonylurea is selected from glibenclamide (glyburide), glibornuride, gliclazide, glipizide, glimepiride, gliquidone, glisoxepide and glyclopyramide.

In one highly preferred embodiment, the sulfonylurea is selected from glibenclamide, and structural and functional analogues thereof.

Preferably, the sulfonylurea is selected from acylhydrazone, sulfonamide and sulfonylthiourea derivatives of glibenclamide, glimepiride, glipizide and gliclazide.

In one preferred embodiment, the sulfonylurea is glimepiride, which has the structure shown below:

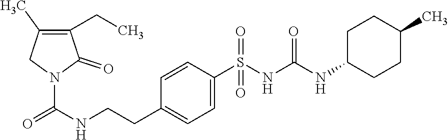

In one preferred embodiment, the sulfonylurea is gliclazide, which has the structure shown below:

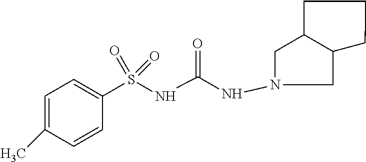 [SMILES: Cc1ccc(S(=O)(=O)NC(=O)NN2CC3CCCC3C2)cc1]

In one preferred embodiment, the sulfonylurea is glipizide, which has the structure shown below:

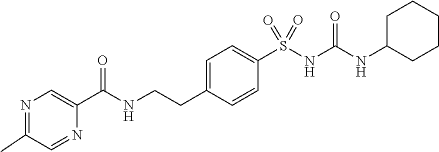 [SMILES: Cc1cnc(C(=O)NCCc2ccc(S(=O)(=O)NC(=O)NC3CCCCC3)cc2)cn1]

In one particularly preferred embodiment, the sulfonylurea is glibenclamide.

Glibenclamide has systematic (IUPAC) name 5-chloro-N-[2-[4-(cyclohexylcarbamoyl-sulfamoyl)phenyl]ethyl]-2-methoxybenzamide (chemical formula C₂₃H₂₈CIN₃O₅S) and its molecular weight is 494; it has the following chemical structure:

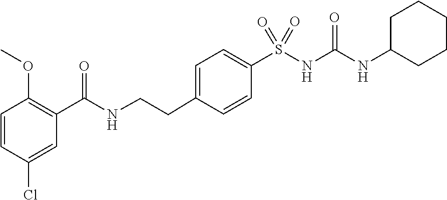 [SMILES: COc1ccc(Cl)cc1C(=O)NCCc1ccc(S(=O)(=O)NC(=O)NC2CCCCC2)cc1]

The invention also encompasses structural and functional analogues of the glibenclamide, particularly those that are modified so as to extend the half-life of the agent, for example, conjugates of glibenclamide.

Glibenclamide (also known as glyburide) is a sulfonylurea receptor-1 (Sur-1) receptor antagonist that is used as a hypoglycaemic agent to treat diabetes. Glibenclamide is being explored as a treatment to reduce oedema after brain injuries, such as ischemic stroke, traumatic brain injury, and subarachnoid haemorrhage, but the results so far are inconsistent (Wilkinson C M, et al. PLoS One. 2019; 14: e0215952; Xu F, et al. Brain Behav. 2019; 9(4): e01254; King Z A, et al. Drug Des Devel Ther. 2018; 12: 2539-2552). The present inventors investigated the role of glibenclamide as part of a combination therapy aiming to reduce reperfusion injury and leading potentially to a neuroprotective effect.

Glibenclamide is available as a generic and is sold in doses of 1.25, 2.5 and 5 mg under many brand names including Gliben-J, Daonil, Diabeta, Euglucon, Gilemal, Glidanil, Glybovin, Glynase, Maninil, Micronase and Semi-Daonil. Glibenclamide is used orally for the treatment of Type 2 diabetes, as a tablet formulation (for adults) or as an oral suspension (for children). The defined daily dose (DDD) of glibenclamide for the treatment of Type 2 diabetes is 7 mg for the micronized formulation, which has higher bioavailability and 10 mg for the conventional formulation. The defined daily dose (DDD) is the assumed average maintenance dose per day for a drug used for its main indication in adults, as defined in accordance with the WHO Collaborating Centre for Drug Statistics Methodology. The DDD is a unit of measurement and does not necessarily reflect the recommended or Prescribed Daily Dose. Therapeutic doses for individual patients and patient groups will often differ from the DDD as they will be based on individual characteristics (such as age, weight, ethnic differences, type and severity of disease) and pharmacokinetic considerations. The DDD value for glibenclamide is obtained from WHO Collaborating Centre for Drug Statistics Methodology (see http://www.whocc.no/atc_ddd_index/?code=A10BB01&show description=yes). The usual starting dose of glibenclamide (micronized formulation) as initial therapy is 2.5 to 5 mg daily and the usual maintenance dose is in the range of 1.25 to 20 mg daily, which may be given as a single dose or in divided doses, administered with breakfast or the first main meal (in accordance with the FDA label for Micronase® glyburide tablets). This corresponds to a maintenance dose of about 18 μg/kg to about 285 μg/kg for a 70 kg adult.

Several studies in animal models have demonstrated a protective role of glibenclamide in inflammation-associated injury including reduced adverse neuroinflammation and improved behavioral outcomes following central nervous system injury (Zhang G, et al. Mediators Inflamm. 2017; 2017: 3578702) or ischemic and hemorrhagic stroke (Caffes N, et al. Int J Mol Sci. 2015; 16: 4973-84). In a traumatic brain injury model in rats, glibenclamide was administered as loading dose of 10 μg/kg intraperitoneally followed by an infusion of 200 ng/hr for 7 days (Patel A D, et al. J Neuropathol Exp Neurol. 2010; 69: 1177-90), while in a mouse model of traumatic brain injury the dose of glibenclamide was 10 μg for three days after a controlled cortical impact injury (Xu Z M, et al. J Neurotrauma. 2017; 34: 925-933). In rodent models of brain ischemia and reperfusion injury, glibenclamide was shown to be effective at doses of 1 mg/kg administered 10 min before reperfusion (Abdallah D M, et al. Brain Res. 2011; 1385: 257-62). In rodent models of subarachnoid hemorrhage, glibenclamide was shown to be effective when administered as a loading dose of 10 μg/kg intraperitoneally followed by an infusion of 200 ng/hr for 24 hours (Simard, J. M, et al. Journal of Cerebral Blood Flow and Metabolism. 2009; 29; 317-330) or for one week (Tosun C, et al. Stroke. 2013; 44: 3522-8). Glibenclamide administered as a continuous infusion (75 ng/h) reduced cerebral edema, infarct volume and mortality by 50%, with the reduction in infarct volume being associated with cortical sparing, at 7 days post middle cerebral artery occlusion in a rat thromboembolic model of stroke (Simard J M, et al. Nat Med. 2006; 12: 433-40). Glibenclamide, administered at a dose of 10 μg either before or at 2 h after experimental intracerebral hemorrhage in mice, was shown to alleviate cerebral edema, disrupted BBB, and neurological deficit (Xu F, et al. Brain Behav. 2019; 9: e01254) and similar findings were obtained in another study (Jiang B, et al. Transl Stroke Res. 2017; 8: 183-193), but the widely-used glibenclamide dose which has been shown to be effective in other studies (10 μg/kg loading dose followed by 200 ng/h for up to 7 days) was shown to be not effective when the intracerebral hemorrhage was produced by intra-striatal injection of collagenase (Wilkinson C M, et al. PLoS One. 2019; 14(5): e0215952).

Glibenclamide was shown to exert beneficial effects in stroke patients also in some clinical trials. In the Glyburide Advantage in Malignant Edema and Stroke (GAMES) clinical trials in patients with large hemispheric infarctions, glyburide was administered intravenously (RP-1127) as a 0.13 mg bolus intravenous injection for the first 2 min, followed by an infusion of 0.16 mg/h for the first 6 h and then 0.11 mg/h for the remaining 66 h and revealed promising findings with regard to brain swelling (midline shift), MMP-9, functional outcomes and mortality (King Z A, et al. Drug Des Devel Ther. 2018; 12: 2539-2552). In an exploratory study with oral glibenclamide administration in patients with acute hemispheric infarction, treatment was shown to be safe, but it did not substantially improve 6-month functional outcome, although it was associated with lighter brain edema and a slight trend towards less severe disability and death was observed (Huang K, et al. Acta Neurol Scand. 2019 May 29). Retrospective analysis of data on diabetic patients who were not on a sulfonylurea to those who were during the days following acute ischemic strokes found a strong association between sulfonylurea treatment and improved survival, greater functional independence, lower NIH stroke scale scores, and less hemorrhagic transformation (Kunte H, et al. Ann Neurol. 2012; 72: 799-806).

The above preclinical and clinical findings are likely to be related to the upregulation of SUR1-TRPM4 channels after brain injuries such as ischemia (Woo S K, et al. J Biol Chem. 2013; 288: 3655-67; Mehta R I et al. J Neuropathol Exp Neurol. 2015; 74: 835-49).

Neuroprotective effects in animal models of ischemia and reperfusion injury have been reported also for other sulfonylureas, for example for gliclazide (Tan F, et al. Brain Res. 2014; 1560: 83-90), as well as protective effects in animal models of ischemia and reperfusion injury in other tissues, such as the myocardium for glimepiride (Nishida H et al. J Pharmacol Sci. 2009; 109: 251-6).

Some other drugs have insulin-secretagogue effects like the sulfonylureas; examples include the glinides (such as repaglinide, nateglinide and mitiglinide). Furthermore, other compounds, such as resveratrol, have been shown to bind to the sulfonylurea receptor (Hambrock A, et al. J Biol Chem. 2007; 282: 3347-56) and to have neuroprotective effects in stroke and traumatic CNS injury (Lopez M S, et al. Neurochem Int. 2015; 89: 75-82).

Studies by the Applicant, and described in more detail in the accompanying examples, have shown that administering a sulfonyl urea (e.g. glibenclamide) in combination with amodiaquine and an aldosterone antagonist (e.g. potassium canrenoate) leads to a clinical benefit effect, even when the sulfonyl urea is administered at only very low doses.

Insulin Modulator In certain embodiments, the combination or pharmaceutical composition or pharmaceutical product of the invention comprises an insulin modulator.

As used herein the term “insulin modulator” refers to an agent that is capable of directly or indirectly increasing or decreasing the activity of insulin, which in turn may increase or decrease the insulin-mediated physiological response.

In one embodiment, the insulin modulator is selected from GLP-1 agonists, DPP-4 inhibitors, PPAR agonists, insulin and analogues thereof.

Examples of GLP-1 agonists include exenatide, lixisenatide, albiglutide, semaglutide, liraglutide, taspoglutide and dulaglutide (LY2189265) and pharmaceutically acceptable salts thereof.

Examples of DPP-4 inhibitors include sitagliptin, vildagliptin, saxagliptin, linagliptin anagliptin, teneligliptin, alogliptin, trelagliptin, gemigliptin, dutogliptin and omarigliptin (MK-3102) and pharmaceutically acceptable salts thereof.

Examples of PPAR agonists include clofibrate, gemfibrozil, ciprofibrate, bezafibrate, fenofibrate, saroglitazar, aleglitazar, muraglitazar and tesaglitazar and pharmaceutically acceptable salts thereof.

Examples of insulin analogues include insulin lispro, insulin aspart, insulin glulisine, insulin detemir, insulin degludec, insulin glargine and pharmaceutically acceptable salts thereof.

Accordingly, in one embodiment the insulin modulator is selected from exenatide, lixisenatide, albiglutide, semaglutide, liraglutide, taspoglutide, dulaglutide (LY2189265), sitagliptin, vildagliptin, saxagliptin, linagliptin anagliptin, teneligliptin, alogliptin, trelagliptin, gemigliptin, dutogliptin, omarigliptin (MK-3102), clofibrate, gemfibrozil, ciprofibrate, bezafibrate, fenofibrate, saroglitazar, aleglitazar, muraglitazar tesaglitazar, insulin lispro, insulin aspart, insulin glulisine, insulin detemir, insulin degludec, insulin glargine and pharmaceutically acceptable salts thereof.

In one embodiment, the insulin modulator is a GLP-1 agonist selected from exenatide, lixisenatide, albiglutide, semaglutide, liraglutide, taspoglutide, dulaglutide (LY2189265) and pharmaceutically acceptable salts thereof. Preferably, the GLP-1 agonist is exenatide.

### Exenatide

In one particularly preferred embodiment, the insulin modulator is selected from exenatide and structural and functional analogues thereof, and pharmaceutically acceptable salts thereof.

In one preferred embodiment, the exenatide is in the form of a pharmaceutically acceptable salt, more preferably, exenatide acetate. In another preferred embodiment, the exenatide is in free base form.

As used herein, the term “exenatide” refers to a 39-mer peptide of the following sequence:

H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂

Exenatide (synonym is exendin 4) is originally isolated from the saliva of the Gila monster, Heloderma suspectum, by Eng in 1992. It is an insulin secretagogue with glucoregulatory effects similar to the human peptide glucagon-like peptide-1 (GLP-1).

Exenatide mimics human glucagon-like peptide 1 (GLP-1), a gut incretin hormone that is release in response to nutrient intake (Goke et al., J. Biol. Chem., 1993, 268: 19650-19655). It exerts insulinotropic and insulinomimetic properties via the GLP-1 receptor. GLP-1 receptor is widely expressed in many organs, including heart and vascular endothelium (Bullock et al., Endocrinology, 1996, 137: 2968-2978; Nystrom et al., Am J Physiol Endocrinol Metab, 2004, 287: E1209-E1215). Currently, exenatide is approved as an anti-diabetic drug for the treatment of patients with diabetes mellitus type 2. The recommended dose in this indication is initially 5 μg (μg) twice daily, increasing to 10 μg twice daily after 1 month based on clinical response.

GLP-1 is ineffective as a therapeutic agent as it has a very short circulating half-life (less than 2 minutes) due to rapid degradation by dipeptidyl peptidase-4. Exenatide is 50% homologous to GLP-1, but has a 2.4 hour half-life in humans as the dipeptidyl peprtidase-4 cleavage site is absent.

Exenatide enhances glucose-dependent insulin secretion by the pancreatic beta-cell, suppresses inappropriately elevated glucagon secretion, and slows gastric emptying. Exenatide is extremely potent, having a minimum effective concentration of 50 μg/mL (12 pM) in humans. Current therapies with exenatide involve twice-daily injections (Byetta®). Also, a slow-release formulation (Bydureon®) has been approved for once-weekly injection.

As used herein a functional analogue of exenatide refers to a compound having a similar structure, but differing from it in a respect of certain aspects (e.g. it can differ in one or more atoms, functional groups, amino acids residues, or substructures, which are replaced with others). Functional analogues display similar pharmacological properties and may be structurally related.

In one embodiment, the structural or functional analogue of exenatide is a form of exenatide that is modified so as to extend the half-life, for example, conjugates of exenatide.

In one preferred embodiment, the structural or functional analogue of exenatide is PEGylated exenatide. For example, in one preferred embodiment, the structural or functional analogue is exenatide mono-PEGylated with 40 kDa PEG. PEGylated exenatide can be prepared by methods known in the art. By way of example, PEGylated forms of exenatide are described in WO 2013/059323 (Prolynx LLC), the contents of which are hereby incorporated by reference. Exenatide can also be conjugated to other molecules, e.g. proteins.

In one particularly preferred embodiment, the structural or functional analogue of exenatide is an extended release form, for example, that marketed under the tradename Bydureon®. In another preferred embodiment, the structural or functional analogue of exenatide is in the form of multilayer nanoparticles for sustained delivery, for example, as described in Kim J Y et al, Biomaterials, 2013; 34:8444-9, the contents of which are hereby incorporated by reference.

In another particularly preferred embodiment, the exenatide is in an injectable form such as that marketed under the tradename Byetta®.

Functional analogues of exenatide include GLP receptor agonists. Suitable functional analogues of exenatide include lixisenatide, albiglutide, semaglutide, liraglutide, taspoglutide and dulaglutide (LY2189265).

In one embodiment, functional analogues of exenatide include exenatide modified wherein one or more amino acid residues has been exchanged for another amino acid residue and/or wherein one or more amino acid residues have been deleted and/or wherein one or more amino acid residues have been added and/or inserted.

In one embodiment, a functional exenatide analogue comprises less than 10 amino acid modifications (substitutions, deletions, additions (including insertions) and any combination thereof) relative to exenatide, alternatively less than 9, 8, 7, 6, 5, 4, 3 or 2 modifications relative to exenatide.

In one embodiment, a functional exenatide analogue comprises 10 amino acid modifications (substitutions, deletions, additions (including insertions) and any combination thereof) relative to exenatide, alternatively 9, 8, 7, 6, 5, 4, 3, 2 or 1 modifications relative to exenatide.

Structural and functional analogues of exenatide also include salts, isomers, enantiomers, solvates, polymorphs, prodrugs and metabolites thereof.

### Aldosterone Antagonist

In certain embodiments, the combination or pharmaceutical composition or pharmaceutical product of the invention comprises an aldosterone antagonist.

Acute myocardial infarction and its subsequent hemodynamic changes lead to complex neurohormonal activation. The renin-angiotensin-aldosterone pathway is one cornerstone of such neurohormonal activation. Aldosterone, which is at its highest levels at presentation after acute myocardial infarction, is reported to promote a broad spectrum of deleterious cardiovascular effects including acute endothelial dysfunction, inhibition of NO activity, increased endothelial oxidative stress, increased vascular tone, inhibition of tissue recapture of catecholamines, rapid occurrence of vascular smooth muscle cell and cardiac myocyte necrosis, collagen deposition in blood vessels, myocardial hypertrophy, and fibrosis (Struthers, Am Heart J, 2002, 144: S2-S7; Zannad and Radauceanu, Heart Fail Rev, 2005, 10: 71-78). Furthermore, it has been found to predict poor outcomes (Beygui et al, Circulation, 2006, 114: 2604-2610). An aldosterone antagonist or an antimineralocorticoid, is a diuretic drug which antagonizes the action of aldosterone at mineralocorticoid receptors. This group of drugs is often used for the management of chronic heart failure. Members of this class are also used in the management of hyperaldosteronism (including Conn's syndrome) and female hirsutism (due to additional antiandrogen actions). Most antimineralocorticoids are steroidal spirolactones.

Antagonism of mineralocorticoid receptors inhibits sodium resorption in the collecting duct of the nephron in the kidneys. This interferes with sodium/potassium exchange, reducing urinary potassium excretion and weakly increasing water excretion (diuresis). In congestive heart failure, aldosterone antagonists are used in addition to other drugs for additive diuretic effect, which reduces edema and the cardiac workload.

Current guidelines recommend the use of mineralocorticoid receptor antagonists, in patients presenting with heart failure post myocardial infarction, based on the results of the EPHESUS trial.

Several studies in animal models of acute myocardial infarction and in the clinic have shown the benefit of aldosterone blockade in the prevention of reperfusion injury and improving heart function in STEMI patient. There are indications in the literature that mineralocorticoid receptor antagonists may have beneficial effects in the cerebral vasculature and during stroke (Dinh Q N, et al. Neural Regen Res. 2016; 11:1230-1).

Examples of aldosterone antagonists for use in the combinations of the invention include spironolactone (the first and most widely used member of this class), eplerenone (much more selective than spironolactone on target, but somewhat less potent and efficacious), canrenone and potassium canrenoate, finerenone (non-steroidal and more potent and selective than either eplerenone or spironolactone) and prorenone. Some drugs also have antimineralocorticoid effects secondary to their main mechanism of actions. Examples include progesterone, drospirenone, gestodene, and benidipine.

In one particularly preferred embodiment, the aldosterone antagonist is potassium canrenoate.

The invention also encompasses structural and functional analogues of aldosterone antagonists, particularly those that are modified so as to extend the half life of the agent, for example, conjugates of aldosterone antagonists.

### Potassium Canrenoate

Potassium canrenoate or canrenoate potassium also known as the potassium salt of canrenoic acid, is an aldosterone antagonist of the spirolactone group. Like spironolactone, it is a prodrug, which is metabolized to canrenone in the body.

Potassium canrenoate is typically given intravenously at doses ranging between 200 mg/day and 600 mg/day for the treatment of hyperaldosteronism or hypokaliaemia.

Potassium canrenoate has the systematic (IUPAC) name potassium 3-[(8R,9S,10R,13S,14S,17R)-17-hydroxy-10,13-dimethyl-3-oxo-2,8,9,11,12,14,15,16-octahydro-1H-cyclopenta[a]phenanthren-17-yl]propanoate, formula C₂₂H₂₉KO₄ and the following chemical structure:

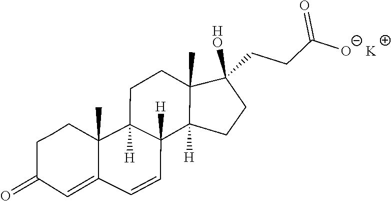

### Combinations

In one aspect, the present invention relates to a combination comprising, or consisting essentially of, or consisting of:
(a) a first component which is a Nurr1 agonist; and
(b) at least one additional component selected from:
   (i) an aldosterone antagonist;
   (ii) an insulin modulator; and
   (iii) a sulfonylurea.

The preferred embodiments described below apply mutatis mutandis to other aspects of the invention, including methods, uses, products and compositions.

In one embodiment, the insulin modulator is defined according to any of the above-mentioned embodiments of an insulin modulator.

In one embodiment, the aldosterone antagonist is defined according to any of the above mentioned embodiments of an aldosterone antagonist.

In one embodiment, the sulfonylurea is defined according to any of the above mentioned embodiments of a sulfonylurea.

In one embodiment, the present invention relates to a combination comprising, or consisting essentially of, or consisting of:
amodiaquine, or a pharmaceutically acceptable salt thereof;
an aldosterone antagonist; and
at least one of the following components: an insulin modulator, and a sulfonylurea.

In one embodiment, the present invention relates to a combination comprising, or consisting essentially of, or consisting of:
amodiaquine, or a pharmaceutically acceptable salt thereof;
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
at least one of exenatide, lixisenatide, albiglutide, semaglutide, liraglutide, taspoglutide and dulaglutide (LY2189265) or pharmaceutically acceptable salts thereof, and
at least one of potassium canrenoate, canrenone, spironolactone, eplerenone, finerenone and prorenone or pharmaceutically acceptable salts thereof, where applicable.

In another embodiment, the present invention relates to a combination comprising, or consisting essentially of, or consisting of:
amodiaquine, or a pharmaceutically acceptable salt thereof;
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
at least one of potassium canrenoate, canrenone, spironolactone, eplerenone, finerenone and prorenone or pharmaceutically acceptable salts thereof where applicable.

In one embodiment, the present invention relates to a combination comprising, or consisting essentially of, or consisting of:
amodiaquine, or a pharmaceutically acceptable salt thereof;
at least one of exenatide, lixisenatide, albiglutide, semaglutide, liraglutide, taspoglutide and dulaglutide (LY2189265) or pharmaceutically acceptable salts thereof, and
at least one of potassium canrenoate, canrenone, spironolactone, eplerenone, finerenone and prorenone or pharmaceutically acceptable salts thereof, where applicable.

In one embodiment, the present invention relates to a combination comprising, or consisting essentially of, or consisting of:
amodiaquine, or a pharmaceutically acceptable salt thereof; and
at least one of exenatide, lixisenatide, albiglutide, semaglutide, liraglutide, taspoglutide and dulaglutide (LY2189265) or pharmaceutically acceptable salts thereof.

In one embodiment, the present invention relates to a combination of, or consisting of, or consisting essentially of, or comprising:
amodiaquine, or a pharmaceutically acceptable salt thereof; and
exenatide or a pharmaceutically acceptable salt thereof.

In one embodiment, the present invention relates to a combination of, or consisting of, or consisting essentially of, or comprising:
amodiaquine, or a pharmaceutically acceptable salt thereof;
potassium canrenoate, and
at least one of glibenclamide glibornuride, gliclazide, glipizide, glimepiride, gliquidone, glisoxepide, glyclopyramide, chlorpropamide, tolbutamide, acetohexamide, carbutamide, glycyclamide, tolhexamide, metahexamide, and tolazamide.

In one embodiment, the present invention relates to a combination of, or consisting of, or consisting essentially of, or comprising:
amodiaquine, or a pharmaceutically acceptable salt thereof;
potassium canrenoate, and
at least one of glibenclamide, glibornuride, gliclazide, glipizide, glimepiride, gliquidone, glisoxepide and glyclopyramide.

In one embodiment, the present invention relates to a combination of, or comprising:
amodiaquine, or a pharmaceutically acceptable salt thereof;
at least one of potassium canrenoate, canrenone, spironolactone, eplerenone, finerenone and prorenone or pharmaceutically acceptable salts thereof where applicable, and
glibenclamide.

In one embodiment, the present invention relates to a combination of, or consisting of, or consisting essentially of, or comprising:
amodiaquine;
potassium canrenoate; and
glibenclamide.

In one embodiment, the present invention relates to a combination comprising: amodiaquine, or a pharmaceutically acceptable salt thereof;
at least one of exenatide, lixisenatide, albiglutide, semaglutide, liraglutide, taspoglutide and dulaglutide (LY2189265) or pharmaceutically acceptable salts thereof, and
at least one of potassium canrenoate, canrenone, spironolactone, eplerenone, finerenone and prorenone or pharmaceutically acceptable salts thereof, where applicable.

In one embodiment, the present invention relates to a combination comprising:
amodiaquine, or a pharmaceutically acceptable salt thereof;
at least one of exenatide, lixisenatide, albiglutide, semaglutide, liraglutide, taspoglutide and dulaglutide (LY2189265) or pharmaceutically acceptable salts thereof; and
potassium canrenoate.

In one embodiment, the present invention relates to a combination comprising:
amodiaquine, or a pharmaceutically acceptable salt thereof;
exenatide or a pharmaceutically acceptable salt thereof; and
at least one of potassium canrenoate, canrenone, spironolactone, eplerenone, finerenone and prorenone or pharmaceutically acceptable salts thereof, where applicable.

In one embodiment, the present invention relates to a combination of, or comprising:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

In one embodiment, the present invention relates to a combination of, or consisting of, or consisting essentially of, or comprising:
amodiaquine, or a pharmaceutically acceptable salt thereof;
exenatide or a pharmaceutically acceptable salt thereof; and
glibenclamide.

In one embodiment, the present invention relates to a combination of, or consisting of, or consisting essentially of, or comprising:
amodiaquine, or a pharmaceutically acceptable salt thereof;
exenatide or a pharmaceutically acceptable salt thereof; and
potassium canrenoate,

In one embodiment, the present invention relates to a combination of, or consisting of, or consisting essentially of, or comprising:
amodiaquine, or a pharmaceutically acceptable salt thereof;
exenatide or a pharmaceutically acceptable salt thereof
potassium canrenoate; and
glibenclamide.

In one aspect of the invention, for each of the above embodiments, the combination consists of the Nurr1 agonist (e.g. amodiaquine or pharmaceutically acceptable salt thereof) and the sulfonyl urea and/or the aldosterone antagonist and/or insulin modulator, i.e. these are the only active agents present. In another (alternative) aspect, the combination may further comprise one or more additional active agents as described hereinafter.

In each of the above embodiments, in addition to the active agents, the combination optionally further comprises one more pharmaceutically acceptable diluents, carriers or excipients.

As used herein, the term “consisting essentially of” means that specific further components can be present, namely those not materially affecting the essential characteristics of the combination or composition.

In one preferred embodiment, no other pharmaceutically active agents are present in the combination or pharmaceutical composition or pharmaceutical product according to the invention, i.e. the only pharmaceutically active agents present are a Nurr1 agonist, and at least one additional component selected from: an aldosterone antagonist; an insulin modulator; and a sulfonylurea. The combination, may however, optionally further comprise other inactive ingredients, for example, one or more pharmaceutically acceptable diluents, carriers or excipients.

Thus, in one preferred embodiment, the invention relates to a pharmaceutical product or a pharmaceutical composition consisting of:
(a) a first component which is a Nurr1 agonist; and
(b) at least one additional component selected from:
   (i) an aldosterone antagonist;
   (ii) an insulin modulator; and
   (iii) a sulfonylurea; and
(c) a pharmaceutically acceptable diluent, excipient or carrier.

Preferred embodiments apply mutatis mutandis as for the first aspect, for example, in relation to the choice and permutation of actives.

The effect of drug combinations is inherently unpredictable and there is often a propensity for one drug to partially or completely inhibit the effects of the other. The present invention demonstrates that a combination comprising a Nurr1 agonist (e.g. amodiaquine) and at least one additional active component selected from a sulfonylurea (e.g. glibenclamide), an insulin modulator (e.g. exenatide) and an aldosterone antagonist (e.g. potassium canrenoate), when administered simultaneously, separately or sequentially, does not lead to any significant or dramatic adverse interaction between the two agents. The unexpected absence of any such antagonistic interaction is critical for clinical applications of the combination.

Moreover, preferred combinations according to the invention surprisingly demonstrate a potentiation of the effect of the individual components, such that the optimal doses for the agents is lower than the doses recommended in the approved indications for these agents, and/or also lower than the doses reported in the literature.

In one embodiment, the combinations of the active agents of the present invention produce an enhanced effect as compared to each drug administered alone.

Thus, in one preferred embodiment, the combination is synergistic, i.e. at least two of the actives interact in a synergistic (i.e. greater than additive) manner.

By way of illustration, studies by the Applicant have shown that the preferred doses of glibenclamide in the context of the presently claimed combinations are significantly lower than the doses previously reported in the literature for blood glucose lowering (e.g. diabetes mellitus). In fact, the preferred doses of glibenclamide used in the presently claimed combinations are approximately ˜20 to 285-fold less than the recommended maintenance dose of glibenclamide (micronized formulation) for treating diabetes mellitus (for the micronized formulation of glibenclamide, the recommended maintenance daily dose is 1.25 to 20 mg, which corresponds to 18 μg/kg to 285 μg/kg for a 70 kg adult—contrast with the preferred doses of glibenclamide required in the presently claimed combination treatment, which can be as low as 1 μg/kg body weight). Advantageously, using glibenclamide in these preferred lower doses avoids any effect on blood glucose levels which could otherwise lead to adverse side effects. Studies by the Applicant have also shown that clinically effective doses of glibenclamide as a double or triple combination according to the invention with low doses of exenatide and/or potassium canrenoate are also significantly lower than the doses of glibenclamide shown to be neuroprotective (continuous infusions of 0.16 or 0.11 mg/h, that is 3.84 mg or 2.64 mg daily) in clinical studies published in the literature (see King Z A et al).

Furthermore, in another embodiment, the combinations of the active agents of the present invention produce unexpected synergistic effects as demonstrated by a rat model of transient middle cerebral artery occlusion.

A combination of two or more drugs may lead to different types of drug interaction. A drug interaction is said to be additive when the combined effect of two drugs equals the sum of the effect of each agent given alone. A drug interaction is said to be synergistic if the combined effect of the two drugs exceeds the effects of each drug given alone (Goodman and Gilmans “The Pharmacological Basis of Therapeutics”, 12th Edition).

Combination therapy is an important treatment modality in many disease settings, including cardiovascular disease, cancer and infectious diseases. Recent scientific advances have increased the understanding of the pathophysiological processes that underlie these and other complex diseases. This increased understanding has provided further impetus to develop new therapeutic approaches using combinations of drugs directed at multiple therapeutic targets to improve treatment response, minimize development of resistance, or minimize adverse events. In settings in which combination therapy provides significant therapeutic advantages, there is growing interest in the development of new combinations of two or more drugs.

Advantageously, a synergistic combination may allow for lower doses of each component to be present, thereby decreasing the toxicity of therapy, whilst producing and/or maintaining the same therapeutic effect or an enhanced therapeutic effect. Thus, in a particularly preferred embodiment, each component of the combination is present in a sub-therapeutic amount. The term “sub-therapeutically effective amount” means an amount that is lower than that typically required to produce a therapeutic effect with respect to treatment with each agent alone.

In one embodiment, the present invention relates to a synergistic combination comprising, or consisting of, a Nurr1 agonist or a pharmaceutically acceptable salt thereof and an insulin modulator, optionally further comprising one or more pharmaceutically acceptable diluents, excipients and/or carriers.

In one embodiment, the present invention relates to a synergistic combination comprising, or consisting of, amodiaquine or a pharmaceutically acceptable salt thereof and an insulin modulator, optionally further comprising one or more pharmaceutically acceptable diluents, excipients and/or carriers.

In one embodiment, the present invention relates to a synergistic combination comprising, or consisting of, a Nurr1 agonist or a pharmaceutically acceptable salt thereof, and a sulfonyl urea, optionally further comprising one or more pharmaceutically acceptable diluents, excipients and/or carriers.

In one embodiment, the present invention relates to a synergistic combination comprising, or consisting of, amodiaquine or a pharmaceutically acceptable salt thereof, and a sulfonyl urea, optionally further comprising one or more pharmaceutically acceptable diluents, excipients and/or carriers.

In another embodiment, the invention relates to a synergistic combination comprising, or consisting of, a Nurr1 agonist, a sulfonyl urea, and an aldosterone antagonist, optionally further comprising one or more pharmaceutically acceptable diluents, excipients and/or carriers.

In another embodiment, the invention relates to a synergistic combination comprising, or consisting of, amodiaquine or a pharmaceutically acceptable salt thereof, a sulfonyl urea, and an aldosterone antagonist, optionally further comprising one or more pharmaceutically acceptable diluents, excipients and/or carriers.

In another embodiment, the invention relates to a synergistic combination comprising, or consisting of, a Nurr1 agonist, an insulin modulator, and an aldosterone antagonist, optionally further comprising one or more pharmaceutically acceptable diluents, excipients and/or carriers.

In another embodiment, the invention relates to a synergistic combination comprising, or consisting of, amodiaquine or a pharmaceutically acceptable salt thereof, an insulin modulator, and an aldosterone antagonist, optionally further comprising one or more pharmaceutically acceptable diluents, excipients and/or carriers.

In another embodiment, the invention relates to a synergistic combination comprising, or consisting of, a Nurr1 agonist, an insulin modulator, a sulfonyl urea, and an aldosterone antagonist, optionally further comprising one or more pharmaceutically acceptable diluents, excipients and/or carriers.

In another embodiment, the invention relates to a synergistic combination comprising, or consisting of, amodiaquine or a pharmaceutically acceptable salt thereof, an insulin modulator, a sulfonyl urea, and an aldosterone antagonist, optionally further comprising one or more pharmaceutically acceptable diluents, excipients and/or carriers.

In one embodiment, the insulin modulator is defined according to any of the above mentioned embodiments of an insulin modulator.

In one embodiment, the aldosterone antagonist is defined according to any of the above mentioned embodiments of an aldosterone antagonist.

In one embodiment, the sulfonyl urea is defined according to any of the above mentioned embodiments of sulfonyl urea.

In one embodiment, the present invention relates to a synergistic combination comprising, or consisting of, amodiaquine or a pharmaceutically acceptable salt thereof, a sulfonylurea, and at least one of potassium canrenoate, canrenone, spironolactone, eplerenone, finerenone and prorenone or pharmaceutically acceptable salts thereof, where applicable.

In one embodiment, the present invention relates to a synergistic combination comprising, or consisting of:
amodiaquine or a pharmaceutically acceptable salt thereof;
at least one of glibenclamide, glibornuride, gliclazide, glipizide, glimepiride, gliquidone, glisoxepide, and glyclopyramide;
at least one of exenatide, lixisenatide, albiglutide, semaglutide, liraglutide, taspoglutide and dulaglutide (LY2189265) or a pharmaceutically acceptable salt thereof; and
potassium canrenoate.

In one embodiment, the present invention relates to a synergistic combination comprising, or consisting of:
amodiaquine or a pharmaceutically acceptable salt thereof;
at least one of glibenclamide, glibornuride, gliclazide, glipizide, glimepiride, gliquidone, glisoxepide, and glyclopyramide; and
exenatide, or a pharmaceutically acceptable salt thereof; and
potassium canrenoate.

In one embodiment, the present invention relates to a synergistic combination comprising, or consisting of:
amodiaquine or a pharmaceutically acceptable salt thereof;
at least one of potassium canrenoate, canrenone, spironolactone, eplerenone, finerenone and prorenone or pharmaceutically acceptable salts thereof;
exenatide or a pharmaceutically acceptable salt thereof; and
at least one of glibenclamide, glibornuride, gliclazide, glipizide, glimepiride, gliquidone, glisoxepide, and glyclopyramide.

In one embodiment, the present invention relates to a synergistic combination comprising, or consisting of:
amodiaquine or a pharmaceutically acceptable salt thereof;
at least one of potassium canrenoate, canrenone, spironolactone, eplerenone, finerenone and prorenone or pharmaceutically acceptable salts thereof; and
exenatide or a pharmaceutically acceptable salt thereof.

In one embodiment, the present invention relates to a synergistic combination comprising, or consisting of:
amodiaquine or a pharmaceutically acceptable salt thereof;
potassium canrenoate; and
at least one of exenatide, lixisenatide, albiglutide, semaglutide, liraglutide, taspoglutide and dulaglutide (LY2189265) or a pharmaceutically acceptable salt thereof.

In one embodiment, the present invention relates to a synergistic combination comprising, or consisting of:
amodiaquine or a pharmaceutically acceptable salt thereof;
potassium canrenoate; and
at least one of glibenclamide, glibornuride, gliclazide, glipizide, glimepiride, gliquidone, glisoxepide, and glyclopyramide.

In one embodiment, the present invention relates to a synergistic combination comprising, or consisting of:
amodiaquine or a pharmaceutically acceptable salt thereof;
at least one of potassium canrenoate, canrenone, spironolactone, eplerenone, finerenone and prorenone or pharmaceutically acceptable salts thereof; and
glibenclamide.

In one embodiment, the present invention relates to a synergistic combination comprising, or consisting of:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

In one embodiment, the present invention relates to a synergistic combination comprising, or consisting of, amodiaquine or a pharmaceutically acceptable salt thereof, and exenatide or a pharmaceutically acceptable salt thereof.

In one embodiment, the present invention relates to a synergistic combination comprising, or consisting of, amodiaquine or a pharmaceutically acceptable salt thereof, exenatide or a pharmaceutically acceptable salt thereof, and potassium canrenoate.

In one embodiment, the present invention relates to a synergistic combination comprising, or consisting of, amodiaquine or a pharmaceutically acceptable salt thereof, potassium canrenoate and glibeclamide.

In one embodiment, the present invention relates to a synergistic combination comprising, or consisting of: amodiaquine, or a pharmaceutically acceptable salt thereof, exenatide or a pharmaceutically acceptable salt thereof, and glibenclamide.

In one embodiment, the present invention relates to a synergistic combination comprising, or consisting of, amodiaquine or a pharmaceutically acceptable salt thereof, potassium canrenoate, glibeclamide and exenatide or a pharmaceutically acceptable salt thereof.

### Additional Active Pharmaceutical Ingredients

In one embodiment, the above described combinations, pharmaceutical compositions and pharmaceutical products comprise at least one further active pharmaceutical ingredient (API).

In one embodiment, the above described combinations may further comprise at least one further API selected from a beta blocker, a renin-angiotensin inhibitor, a statin (HMG-CoA reductase inhibitor), an inhibitor of platelet activation or aggregation, a phosphodiesterase-3 inhibitor, a calcium sensitizer, an antioxidant, and an anti-inflammatory agent.

Examples of beta-blockers include propranolol, metoprolol, bucindolol, carteolol, carvedilol, labetalol, nadolol, oxprenolol, penbutolol, pindolol, sotalol and timolol.

Renin-angiotensin inhibitors include angiotensin converting enzyme inhibitors, angiotensin AT₁ receptor inhibitors and renin inhibitors.

Examples of angiotensin converting enzyme inhibitors include captopril, zofenopril, enalapril, ramipril, quinapril, perindopril, lisinopril, benazepril, imidapril, trandolapril, cilazapril, and fosinopril.

Examples of angiotension AT₁ receptor antagonists include losartan, irbesartan, olmesartan, candesartan, valsartan, fimasartan and telmisartan.

Examples of renin inhibitors include remikiren and aliskiren.

Examples of calcium sensitizers include levosimendan and analogues thereof.

Examples of statins include atorvastatin, lovastatin, pravastatin, rosuvastatin and simvastatin.

Examples of platelet activation or aggregation inhibitors include prostacyclin (epoprostenol) and structural and functional analogues thereof (eg. treprostinil, iloprost), irreversible cyclooxygenase inhibitors (e.g. Aspirin, Triflusal), adenosine diphosphate (ADP) receptor inhibitors (e.g. Clopidogrel, Prasugrel, Ticagrelor, Ticlopidine), phosphodiesterase inhibitors (e.g. Cilostazol), protease-activated receptor-1 (PAR-1) antagonists (e.g. Vorapaxar), glycoprotein IIB/IIIA inhibitors (e.g. Abciximab, Eptifibatide, Tirofiban), adenosine reuptake inhibitors (e.g. Dipyridamole), and thromboxane inhibitors, including thromboxane synthase inhibitors and thromboxane receptor antagonists (e.g. Terutroban).

Examples of phosphodiesterase-3 (PDE-3) inhibitors include amrinone, milrinone, and analogues thereof.

Examples of antioxidants include ascorbic acid, lipoic acid, glutathione, melatonin and resveratrol.

Examples of anti-inflammatory agents include COX-2 inhibitors (e.g. celecoxib), glucocorticoids (e.g. hydrocortisone), and non-steroidal anti-inflammatory drugs (e.g. ibuprofen).

In one embodiment, the above combinations comprise at least one further API selected from propranolol, metoprolol, bucindolol, carteolol, carvedilol, labetalol, nadolol, oxprenolol, penbutolol, pindolol, sotalol, timolol, captopril, zofenopril, enalapril, ramipril, quinapril, perindopril, lisinopril, benazepril, imidapril, trandolapril, cilazapril, fosinopril, losartan, irbesartan, olmesartan, candesartan, valsartan, fimasartan, telmisartan, remikiren, aliskiren, melatonin and resveratrol.

In another embodiment, the above combinations comprise at least one further API selected from carvedilol, metoprolol, losartan, irbesartan, olmesartan, candesartan, valsartan, fimasartan telmisartan. captopril, zofenopril, enalapril, ramipril, quinapril, perindopril, lisinopril, benazepril, imidapril, trandolapril, cilazapril, fosinopril, remikiren aliskiren, melatonin and resveratrol.

In another embodiment, the above combinations comprise at least one further API selected from carvedilol, metoprolol, melatonin and resveratrol.

### Pharmaceutically Acceptable Salts

The active pharmaceutical agents of the present invention can be present as pharmaceutically acceptable salts.

Pharmaceutically acceptable salts of the agents of the invention include suitable acid addition or base salts thereof. A review of suitable pharmaceutical salts may be found in Berge et al., J Pharm Sci, 66, 1-19 (1977). Salts are formed, for example with strong inorganic acids such as mineral acids, e.g. sulphuric acid, phosphoric acid or hydrohalic acids (e.g. HCl, HBr); with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl- or aryl-sulfonic acids which are unsubstituted or substituted (for example, by a halogen) such as methane- or p-toluene sulfonic acid.

### Enantiomers/Tautomers

The invention also includes where appropriate all enantiomers and tautomers of the active pharmaceutical agents. The person skilled in the art will recognise compounds that possess optical properties (one or more chiral carbon atoms) or tautomeric characteristics. The corresponding enantiomers and/or tautomers may be isolated/prepared by methods known in the art.

### Stereo and Geometric Isomers

Some of the active pharmaceutical agents of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the active pharmaceutical agents or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as 2H, 3H, 13C, 14C, 15N, 17O, 18O, 31P, 32P, 35S, 18F and 36Cl, respectively. Certain isotopic variations of the agent and pharmaceutically acceptable salts thereof, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability.

Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes solvate forms of the active pharmaceutical agents of the present invention. The terms used in the claims encompass these forms.

### Polymorphs

The invention furthermore relates to active pharmaceutical agents of the present invention in their various crystalline forms, polymorphic forms and (an)hydrous forms. It is well established within the pharmaceutical industry that chemical compounds may be isolated in any of such forms by slightly varying the method of purification and or isolation from the solvents used in the synthetic preparation of such compounds.

### Pharmaceutical Compositions

In another aspect, the present invention relates to a pharmaceutical composition comprising a combination according to the invention as described above and a pharmaceutically acceptable carrier, diluent or excipient.

Even though the compounds of the present invention (including their pharmaceutically acceptable salts) can be administered alone, they will generally be administered in admixture with a pharmaceutical carrier, excipient or diluent, particularly for human therapy. The pharmaceutical compositions may be for human or non-human animal usage in human and veterinary medicine respectively.

Examples of such suitable excipients for the various different forms of pharmaceutical compositions described herein may be found in the “Handbook of Pharmaceutical Excipients”, 2nd Edition, (1994), edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). The carrier, or, if more than one be present, each of the carriers, must be acceptable in the sense of being compatible with the other ingredients of the formulation and not deleterious to the recipient. Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. Examples of routes of administration include parenteral (e.g., intravenous, intramuscular, intradermal, intraperitoneal or subcutaneous), oral, inhalation, transdermal (topical), intraocular, iontophoretic, and transmucosal administration.

The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation.

The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

In one embodiment, the pharmaceutical composition is for oral administration. Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

The active compounds may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility the active compounds may be in the form of a solution or suspension for use in an atomizer or nebuliser whereby an accelerated airstream or ultrasonic agitation is employed to produce a fine droplet mist for inhalation.

In another embodiment, the pharmaceutical composition is for intranasal administration. Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein into conjunction or association with a pharmaceutically or veterinarily acceptable carrier or vehicle.

In one embodiment, the pharmaceutical composition is for parenteral administration (e.g., intravenous, intraarterial, intrathecal, intramuscular, intradermal, intraperitoneal or subcutaneous). Preferably, the compositions are prepared from sterile or sterilisable solutions.

In another embodiment, the pharmaceutical composition is for intravenous, intramuscular, or subcutaneous administration.

In another embodiment, the pharmaceutical composition is for intravenous administration.

Solutions or suspension used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl-alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. The pH can be adjusted with acids or bases, such as hydrochloric acid or sodium hydroxide.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™, or phosphate buffered saline (PBS). In all cases, a composition for parenteral administration must be sterile and should be fluid to the extent that easy syringeability exists. It should be stable under the conditions of manufacture and storage and must be preserved against the contaminating action of microorganisms such as bacteria and fungi.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compounds into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof. The invention also encompasses liposomal and nanoparticulate formulations comprising the active agents.

Such formulations, along with methods for their preparation, will be familiar to a person of ordinary skill in the art.

### Pharmaceutical Products

In another aspect, the present invention relates to a pharmaceutical product comprising, or consisting of:
(a) a first component which is a Nurr1 agonist; and
(b) at least one additional component selected from:
   (i) an aldosterone antagonist;
   (ii) an insulin modulator; and
   (iii) a sulfonylurea.

In one preferred embodiment, the present invention relates to a pharmaceutical product comprising:
amodiaquine, or a pharmaceutically acceptable salt thereof;
an aldosterone antagonist; and
at least one of the following components: an insulin modulator, and a sulfonylurea.

In one preferred embodiment, the present invention relates to a pharmaceutical product consisting of, or consisting essentially of:
amodiaquine, or a pharmaceutically acceptable salt thereof;
an aldosterone antagonist; and
at least one of the following components: an insulin modulator, and a sulfonylurea.

In another aspect, the present invention relates to a pharmaceutical product comprising:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

In another aspect, the present invention relates to a pharmaceutical product consisting of, or consisting essentially of:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

In one preferred embodiment, the present invention relates to a pharmaceutical product comprising:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof.

In one preferred embodiment, the present invention relates to a pharmaceutical product consisting of, or consisting essentially of:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof.

In one preferred embodiment, the present invention relates to a pharmaceutical product comprising:
amodiaquine, or a pharmaceutically acceptable salt thereof;
potassium canrenoate, or a structural or functional analogue thereof;
exenatide, or a structural or functional analogue thereof, or a pharmaceutically acceptable salt thereof; and
glibenclamide, or a structural or functional analogue thereof, or a pharmaceutically acceptable salt thereof.

In one preferred embodiment, the present invention relates to a pharmaceutical product consisting of, or consisting essentially of:
amodiaquine, or a pharmaceutically acceptable salt thereof;
potassium canrenoate, or a structural or functional analogue thereof;
exenatide, or a structural or functional analogue thereof, or a pharmaceutically acceptable salt thereof; and
glibenclamide, or a structural or functional analogue thereof, or a pharmaceutically acceptable salt thereof.

In one preferred embodiment, the present invention relates to a pharmaceutical product comprising:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

In one preferred embodiment, the present invention relates to a pharmaceutical product consisting of, or consisting essentially of:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

In one preferred embodiment, each of the components of the pharmaceutical product is for separate administration.

In one preferred embodiment, each of the components is combined into a single formulation.

In one embodiment, the pharmaceutical product is a kit of parts containing all necessary equipment (e.g., vials of drug, vials of diluent, syringes and needles) for a treatment course.

The components of the kit and pharmaceutical product are as defined above. In a preferred embodiment, each component of the kit or pharmaceutical product is admixed with one or more pharmaceutically acceptable diluents, excipients and/or carriers.

In one embodiment, the kit comprises separate containers for each active agent. Said containers may be ampoules, disposable syringes or multiple dose vials.

In another embodiment, the kit comprises a container which comprises a combined preparation of each active agent.

The kit may further comprise instructions for the treatment and/or prevention of reperfusion injury.

### Therapeutic Uses

The present invention further relates to the above described combination, pharmaceutical product or pharmaceutical composition for use in treating various therapeutic disorders as detailed below, and methods of treatment relating to the same.

One aspect of the invention relates to a combination or a pharmaceutical composition or a pharmaceutical product as described herein for use in the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for use in providing cardioprotection against cardiotoxic drugs, or for use in providing neuroprotection.

In one preferred embodiment, the combination or pharmaceutical composition or pharmaceutical product as described herein is for use in treating or preventing stroke.

In one preferred embodiment, the combination or pharmaceutical composition or pharmaceutical product as described herein is for use in treating or preventing the neurodegenerative consequences of stroke.

Stroke is when poor blood flow to the brain results in cell death. There are two main types of stroke: ischemic, due to lack of blood flow, and haemorrhagic, due to bleeding. They result in part of the brain not functioning properly. Signs and symptoms of a stroke may include an inability to move or feel on one side of the body, problems understanding or speaking, feeling like the world is spinning, or loss of vision to one side among others. An ischemic stroke is typically caused by blockage of a blood vessel. Ischemic stroke treatment includes surgery to open up (reperfusion) the arteries to the brain in those with problematic narrowing. An ischemic stroke, if detected within three to four and half hours, may be treatable with a medication that can break down the clot. In 2013, stroke was the second most frequent cause of death after coronary artery disease, accounting for 6.4 million deaths (12% of the total).

Ischemic stroke and acute myocardial infarction require emergency reperfusion in order to improve functional outcome (Patel and Saver, 2013, Stroke, 44: 94-98). Intravenous tissue-type plasminogen activator has long been the only reperfusion therapy with proven clinical benefit in patients with acute ischemic stroke. As it happens in acute myocardial infarction, endovascular methods restoring reperfusion in acute ischemic stroke may expose patients to increased ischemic/reperfusion injury, thereby hampering the benefit of recanalization by promoting haemorrhagic transformation and severe vasogenic oedema both considering as markers of reperfusion injury (Bai and Lyden. 2015; Int J Stroke, 10: 143-152). Experimental evidence indicates that brain ischemic reperfusion injury (as happens in myocardial reperfusion injury) may be attenuated by ischemic pre- and post-conditioning. Glibenclamide was shown to enhance the therapeutic benefits of early hypothermia after severe stroke in rats (Zhu S, et al. Aging Dis. 2018; 9: 685-695). In addition, in a clinically relevant rat model of stroke (middle cerebral artery occlusion) reperfusion was initiated 4.5 h later and concomitantly was administered recombinant tissue plasminogen activator followed by administration of glibenclamide (10 μg/kg IP loading dose plus 200 ng/h by constant subcutaneous infusion) beginning 4.5 h or 10 h after onset of ischemia; glibenclamide significantly reduced hemispheric swelling at 24 h and 48-h mortality and improved Garcia scores at 48 h suggesting that the treatment window for glibenclamide extends to 10 h after onset of ischemia. This finding is consistent with observations in retrospective clinical studies suggesting that the use of sulfonylureas are beneficial in the context of rt-PA-aided recanalization/reperfusion following acute ischemic stroke (Simard, J M, et al. Ann. N. Y. Acad. Sci. 2012; 1268: 95-107).

In one preferred embodiment, the combination or pharmaceutical composition or pharmaceutical product as described herein is for use in treating or preventing intracerebral haemorrhage (ICH).

In one preferred embodiment, the stroke is a haemorrhagic stroke.

In another preferred embodiment, the stroke is ischemic stroke. Ischemic stroke is one of the most common clinical indications of reperfusion injury.

Studies by the Applicant using a rat model of transient middle cerebral artery occlusion have shown that treatment with combinations according to the invention improve neurological score and significantly reduce infarct size relative to treatment with the vehicle control when administered 20 minutes before perfusion and twice a day thereafter for 7 days (see Example 2). Moreover, the advantageous effects associated with the treatment are observed for an extended period of time. For example, when treated immediately after perfusion and twice daily thereafter for 28 days, triple combination therapy significantly improved neurological score and improved performance in the stepping test and forelimb placement test compared to the vehicle control. Treatment with combinations according to the invention also significantly improved anxiety in the elevated plus maze test during week 2 of the study. Furthermore, after 28 days, improved cognitive function was observed in the object recognition cognitive test, as well as a significant reduction in infarct size (see Example 3).

Prolonged treatment with low dosages of combinations according to the invention therefore provides a new therapeutic approach to the treatment of stroke and other chronic disorders compared to the currently approved short term treatments that are available. Since lesion formation does not cease after stroke, but continues even after the circulation resumes, the long term low dose administration of combinations according to the invention is therapeutically advantageous. At the same time, prolonged low dose administration can minimise the side effects typically associated with conventional short term high dose treatments. The treatment of stroke with combinations according to the present invention is understood to be a two step process. The first step involves minimizing the harmful effects of reperfusion injury, and the second step concerns the immune response after stroke, thereby improving the restoration of brain function.

In one preferred embodiment, the combination or pharmaceutical composition or pharmaceutical product as described herein is for use in treating or preventing neuroinflammation or a neuroinflammatory disorder.

Neuroinflammation is defined as inflammation of neural tissue and may be triggered by a variety of different stimuli. Neuroinflammation is widely regarded as chronic, as opposed to acute, inflammation of the central nervous system (Streit W J et al, July 2004, Journal of Neuroinflammation; 1(1):14). Chronic inflammation involves the sustained activation of glial cells and recruitment of other immune cells into the brain. Common triggers of chronic neuroinflammation include: toxic metabolites, autoimmunity, aging, microbes, viruses, traumatic brain injury, spinal cord injury, air pollution and passive smoke.

Microglia are the innate immune cells of the central nervous system (Gendelman H E, December 2002, Journal of Neurovirology; 8 (6): 474-9) and actively survey their environment through, and change their cell morphology significantly in response to neural injury (Garden G A, October 2013, Neurotherapeutics. 10 (4): 782-8). Astrocytes are glial cells that are involved in maintenance/support of neurons and compose a significant component of the blood-brain barrier. Astrocytes are abundant in the CNS and provide trophic support for neurons, promote formation and function of synapses, and prune synapses by phagocytosis, in addition to fulfilling a range of other homeostatic maintenance functions. After insult to the brain, such as traumatic brain injury, astrocytes may become activated in response to signals released by injured neurons or activated microglia (Mayer C L et al, Headache. 53 (9): 1523-30; Ebert S E et al, Eur J Neurol 2019. doi:10.1111/ene.13971). Once activated, astrocytes may release various growth factors and undergo morphological changes.

Astrocytes are understood to play both a protective and harmful role. Liddelow et al (Nature, 2017, January 26; 541(7638): 481-487) distinguished between two different types of reactive astrocytes termed “A1” and “A2” respectively. Reactive astrocytes induced by ischemia (termed A2 astrocytes) are understood to promote CNS recovery and repair, whilst astrocytes induced by activated microglia in neuroinflammation (termed A1 reactive astrocytes) lose their normal astrocyte function and become neurotoxic.

Liddelow et al disclosed that neurotoxic astrocytes play a key role in the pathological response of the CNS to neuroinflammation, acute CNS injury and many neurodegenerative diseases. After brain injury, or in certain diseases, astrocytes undergo a dramatic transformation called “reactive astrocytosis”, up-regulating many genes and forming a glial scar. A1 reactive astrocytes are induced by activated microglia, losing most of their normal astrocyte functions and gaining a new neurotoxic function, rapidly killing neurons and mature differentiated oligodendrocytes. Liddelow demonstrated that A1s rapidly form in vivo after CNS injury, contributing to neuron death after acute CNS injury. Liddelow further demonstrated that inhibition of A1 reactive astrocyte formation after acute CNS injury was able to prevent death of axotomized neurons.

Histopathological studies by the Applicant have shown that treatment with the triple combination of amodiaquine/potassium canrenoate/glibenclamide led to a statistically significant decrease in NG2 oligodendrocyte progenitor cells compared to the control vehicle. This reduction in activated microglial cells is consistent with a decrease in harmful A1 reactive astroctyes. Further studies by the Applicant have shown that treatment with triple combinations according to the invention reduced GFAP markers (representing reactive astrocytes) and also decreased Iba-1 (microglia cells) compared to the control vehicle, again consistent with a decrease in A1 reactive astrocytes. Studies also showed an increase in neurogenesis as measured by Doublecortin staining. Further details of these experiments are set out in the accompanying Examples (see, in particular, Example 3).

Collectively, these studies indicate that combinations according to the invention have an effect on neuroinflammatory pathways. Without wishing to be bound by theory, the presently claimed combinations may to serve protect cells against neurotoxicity (for example, caused by the build up of toxic substances or prevention of their removal) and/or support repair mechanisms. In the light of the mechanistic pathways described in Liddelow (Nature 2017), the above histopathological results indicate that the combinations described herein have therapeutic applications in the treatment of a variety of neuroinflammatory disorders and/or diseases having a neuroinflammatory component.

Neuroinflammation is also known to play a role in neurodegenerative disorders (Chen et al. 2016). Recent data have identified the inflammatory process, and in particular the role of pro-inflammatory cytokines, as being closely linked to multiple neurodegenerative pathways. In particular, A1 reactive astrocytes are known to be present in many neurodegenerative diseases, including Alzheimer's, Huntingdon's, Parkinson's, ALS and MS (Liddelow et al, 2017). The cellular and molecular mechanisms of neuroinflammation are likely to be the same in aging and metabolic diseases, hypertension, diabetes, depression and dementia or after cerebral insult such as stroke. While multiple mechanisms likely contribute to the etiology and progression of neurodegeneration in Alzheimer's Disease (AD), the pathogenic role of neuroinflammation is now well recognised and accepted (Onyango et al, 2021).

Studies by Kinoshita et al have shown that the Nurr1 agonist amodiaquine attenuates inflammatory events and neurological deficits in a mouse model of intracerebral haemorrhage (Kinoshita et al. 2019). More specifically, studies have shown that the daily administration of amodiaquine (40 mg/kg, i.p.) from 3 h after ICH induction diminished perihematomal activation of microglia/macrophages and astrocytes. Amodiaquine also suppressed ICH-induced mRNA expression of IL-1β, CCL2 and CXCL2, and ameliorated motor dysfunction of mice. Administration of amodiaquine not only attenuated inflammatory responses associated with glial cell activation, but also improved neurological outcome after ICH.

In one preferred embodiment, the neuroinflammatory disorder is selected from Alzheimer's disease, Parkinson's disease, multiple sclerosis, Acute disseminated encephalomyelitis (ADEM), Acute Optic Neuritis (AON), Transverse Myelitis and Neuromyelitis Optica (NMO).

In one preferred embodiment, the neuroinflammation is associated with traumatic brain injury, spinal cord injury, aging, schizophrenia, depression, migraine, epilepsy, neuropathic pain, Down Syndrome, autism, preterm infant, glaucoma, or a viral infection.

Neuroinflammation has an important role in the pathophysiology of migraine, and neuroinflammatory pathways, specifically those involving inflammasome proteins, are promising candidates as treatment targets (Kurson et al. 2021).

In one preferred embodiment, the neuroinflammation is associated with aging. Aging is characterized by a progressive increase in neuroinflammation, which contributes to cognitive impairment, associated with aging and age-related neurodegenerative diseases including Alzheimer's.

In one embodiment, the combination or pharmaceutical composition or pharmaceutical product as described herein is for use in treating an aging-associated disease (commonly termed age-related disease or “ARD”). Essentially, aging-associated diseases are complications arising from senescence and are distinguished from the aging process itself. Examples of aging-associated diseases are atherosclerosis and cardiovascular disease, cancer, arthritis, cataracts, osteoporosis, type 2 diabetes, hypertension and Alzheimer's disease. The incidence of all of these diseases increases exponentially with age.

ARDs are most often seen with increasing frequency with increasing senescence, the phenomenon characterized by the cessation of cell division. Senescence is a cellular programme that imposes a stable arrest on damaged or old cells to avoid their replication. As well as growth arrest, senescent cells undergo profound phenotypic changes that include chromatin reorganisation, increase of β-galactosidase activity (referred to as senescence-associated β-galactosidase or SA-β-Gal) and secretion of multiple factors, mainly pro-inflammatory, that are collectively referred to as the senescence-associated secretory phenotype (SASP). Senescent cells accumulate during the aging process and are associated with many diseases, including cancer, fibrosis and many age-related pathologies. Recent evidence suggests that senescent cells are detrimental in multiple pathologies and their elimination confers many advantages, ameliorating multiple pathologies and increasing healthspan and lifespan.

In one preferred embodiment, the combination or pharmaceutical composition or pharmaceutical product as described herein is for use in treating or preventing a neurodegenerative disease, preferably selected from Parkinson's disease, Alzheimer's disease, Amyotrophic lateral sclerosis, Huntington's disease, amyotrophic lateral sclerosis (ALS) and vascular dementia.

In one preferred embodiment, the neurodegenerative disorder is Parkinson's disease.

In one preferred embodiment, the neurodegenerative disorder is amyotrophic lateral sclerosis (ALS).

In one preferred embodiment, the neurodegenerative disorder is vascular dementia. Studies of the effect of combinations according to the invention in a rat model of vascular dementia are described in more detail in the accompanying examples (see Example 1).

In one preferred embodiment, the neurodegenerative disorder is Alzheimer's disease.

In one preferred embodiment, the combination or pharmaceutical composition or pharmaceutical product as described herein is for use in providing neuroprotection, even more preferably for use in providing neuroprotection against neurotoxic drugs.

As used herein, the term “neuroprotection” refers to protecting a neural entity, including the brain, for example, by preventing, reducing or delaying brain damage that may lead to the death of the neurons and to neurodegenerative disorders such as Alzheimer's disease, Parkinson's disease or vascular dementia.

In one preferred embodiment, the combination or pharmaceutical composition or pharmaceutical product as described herein is for use in providing neuroprotection in stroke.

In one preferred embodiment, the combination or pharmaceutical composition or pharmaceutical product as described herein is for use in providing neuroprotection in neurodegenerative disorders.

In one embodiment, the combination or pharmaceutical composition or pharmaceutical product as described herein is for use in providing neuroprotection in a subject against the neurotoxic effects of drugs. Examples of neurotoxic drugs are described by Gouzoulis-Mayfrank and Daumann (Dialogues Clin Neurosci. 2009; 11(3):305-17). Neurotoxic drugs include drugs of abuse (eg. 3,4-methylendioxymethamphetamine, methamphetamine and amphetamine), pesticides (eg. organic phosphorus-based pesticides), certain chemotherapies (eg. platinum), and dopamine.

In one embodiment, the combination or pharmaceutical composition or pharmaceutical product as described herein is for use in providing cardioprotection in a subject against the cardiotoxic effects of drugs (e.g. anthracyclines). Examples of cardiotoxic drugs are described in Bovelli et al (Annals of Oncology 21 (Supplement 5): v277-v282, 2010).

As used herein, the term “cardioprotection” refers to protecting the heart, for example, by preventing, reducing or delaying myocardial injury. Cardiotoxic drugs include drugs associated with cardiac heart failure, drugs associated with ischaemia or thromboembolism, drugs associated with hypertension, drugs associated with other toxic effects such as tamponade and endomyocardial fibrosis, haemorrhagic myocarditis, bradyarrhythmias, Raynaud's phenomenon, autonomic neuropathy, QT prolongation or torsades de pointes, or pulmonary fibrosis. Examples of cardiotoxic drugs include anthracyclines/anthraquinolones, cyclophosphamide, Trastuzumab and other monoclonal antibody-based tyrosine kinase inhibitors, antimetabolites (fluorouracil, capecitabine), antimicrotubule agents (paclitaxel, docetaxel), cisplatin, thalidomide, bevacizumab, sunitinib, sorafenib, busulfan, paclitaxel, vinblastine, bleomycin, vincristine, arsenic trioxide, bleomycin and methotrexate.

In one preferred embodiment, the combination or pharmaceutical composition or pharmaceutical product as described herein is for use in the treatment and/or prevention of ischemia and/or reperfusion injury.

As used herein, the term “reperfusion injury” refers to the damage to tissue caused when blood supply returns to the tissue after a period of ischemia. The absence of oxygen and nutrients from blood creates a condition in which the restoration of circulation results in inflammation, mitochondrial dysfunction and oxidative damage through the induction of oxidative stress rather than restoration of normal function. Reperfusion injury can occur after a spontaneously occurring event, e.g., arterial blockage, or a planned event, e.g., any of a number of surgical interventions. Myocardial reperfusion injury can occur, for example, after myocardial infarction or as a result of heart transplantation. Cerebral reperfusion injury can occur, for example, after ischemic stroke or as a result of neonatal asphyxia.

In one preferred embodiment, the combination or pharmaceutical composition or pharmaceutical product as described herein is for use in the treatment and/or prevention of reperfusion injury in stroke.

In a more preferred embodiment, the ischemia and/or reperfusion injury is ischemia and/or reperfusion injury of the brain, heart, lung, kidney, preferably cerebral ischemia, cerebral reperfusion injury or stroke.

In one preferred embodiment, the combination or pharmaceutical composition or pharmaceutical product as described herein is for use in treating and/or preventing reperfusion injury of the brain, heart, lung, kidney, or other organ/tissue susceptible to reperfusion injury.

In one embodiment, the combination or pharmaceutical composition or pharmaceutical product as described herein is for use in treating and/or preventing ischemia of the brain, heart, lung, kidney, or other organ/tissue susceptible to ischemia.

In one embodiment, the ischemia and/or reperfusion injury is ischemia and/or reperfusion injury of the brain, preferably cerebral ischemia and/or cerebral reperfusion injury.

In one embodiment, the ischemia and/or reperfusion injury is ischemia and/or reperfusion injury of the heart, preferably myocardial ischemia and/or myocardial reperfusion injury.

In another embodiment, the combination or pharmaceutical composition or pharmaceutical product as described herein is for use in treating and/or preventing reperfusion injury of the brain, preferably cerebral reperfusion injury.

In another embodiment, the combination or pharmaceutical composition or pharmaceutical product as described herein is for use in treating and/or preventing ischemia of the heart, preferably myocardial ischemia.

In one particularly preferred embodiment, the combination or pharmaceutical composition or pharmaceutical product as described herein is for use in treating and/or preventing acute myocardial infarction. Acute myocardial infarction is one of the most common clinical indications of reperfusion injury.

In one embodiment, the combination or pharmaceutical composition or pharmaceutical product as described herein is for use in treating a subject with cardiogenic shock.

Cardiogenic shock is a life-threatening medical condition resulting from an inadequate circulation of blood due to primary failure of the ventricles of the heart to function effectively. The condition occurs in 2-10% of patients hospitalized due to myocardial infarction and is the main cause of death among these patients (Holmes et al, 1995, J Am Coll Cardiol, 26: 668-674). More specifically, cardiogenic shock is the result of a complex process with failure of oxygen delivery, generalized ATP deficiency, and multi-organ dysfunction initiated by cardiac pump failure (Okuda, 2006, Shock, 25: 557-570). As this is a type of circulatory shock, there is insufficient perfusion of tissue to meet the demands for oxygen and nutrients. The condition involves increasingly more pervasive cell death from oxygen starvation (hypoxia) and nutrient starvation (e.g. low blood sugar). Because of this, it may lead to cardiac arrest (or circulatory arrest), which is an abrupt stopping of cardiac pump function (as well as stopped respiration and a loss of consciousness). Cardiogenic shock is defined by sustained low blood pressure with tissue hypoperfusion despite adequate left ventricular filling pressure. Signs of tissue hypoperfusion include low urine production (<30 mL/hour), cool extremities, and altered level of consciousness. Several large trials have demonstrated that coronary revascularization is the most important strategy to improve patient survival (Hochman et al, 1999, N Engl J Med, 341: 625-634). However, patients who develop cardiogenic shock despite acute revascularization have a poor prognosis, likely due to reperfusion injury and considered to be associated to the resulted infarct size. Indeed, hypothermia has shown to offer tissue protection in myocardial ischemia, and preclinical studies have shown beneficial results in reducing infarct size in experimentally induced myocardial infarction (Dae et al, 2002, Am J Physiol Heart Circ Physiol, 282: H1584-H1591). Accordingly, in a pig model mild therapeutic hypothermia reduced acute mortality in cardiogenic shock, and improved hemodynamic parameters (Gotberg et al, 2010, Resuscitation, 81: 1190-96)

In one embodiment, the combination or pharmaceutical composition or pharmaceutical product as described herein is for use in treating a subject with cardiac arrest. Cardiac arrest is a sudden stop in effective blood flow due to the failure of the heart to contract effectively. The most common cause of cardiac arrest is coronary artery disease. Treatment for cardiac arrest is immediate cardiopulmonary resuscitation (CPR) and if a shockable rhythm is present, defibrillation. In the United States cardiac arrest outside of hospital occurs in about 13 per 10,000 people per year (326,000 cases). In hospital cardiac arrest occurs in an additional 209,000 (Kronick et al, Circulation, 2015, 132: S397-S413). In addition to providing high quality cardiopulmonary resuscitation, optimizing the management for post-cardiac arrest syndrome is critically important for improving the long term outcome for cardiac arrest patients. Within this syndrome (“post-cardiac arrest syndrome”) there are 3 major areas of emphasis: (1) post-cardiac arrest brain injury; (2) post-cardiac arrest myocardial dysfunction and reperfusion injury; and (3) systemic ischemia-reperfusion response. It is now clear that post-resuscitation care can affect long-term survival and the myocardial and neurological recovery and function of survivors (Kern, 2015, Circ J, 79: 1156-1163).

In one embodiment the subject is at risk of (or susceptible to) vessel occlusion injury or cardiac ischemia-reperfusion injury.

In one embodiment, the combination or pharmaceutical composition or pharmaceutical product as described herein is administered to a donor subject and/or a recipient subject prior to and/or during and/or after heart transplant. For example, in some embodiments the combination may be administered to a first subject from which the heart organ will be removed for transplantation into a second subject. Additionally or alternatively, in some embodiments, the combination is administered to the extracted heart organ, prior to introduction into the second subject. Additionally or alternatively, in some embodiments, the combination therapy is administered to the second subject before, during and/or after heart transplant.

In one preferred embodiment, the combination or pharmaceutical composition or pharmaceutical product as described herein is for use in treating or preventing neonatal asphyxia.

Neonatal asphyxia (or perinatal asphyxia) is the medical condition resulting from deprivation of oxygen to a newborn infant that lasts long enough during the birth process to cause physical harm, usually to the brain. The most common cause of neonatal asphyxia is a drop in maternal blood pressure or other interference to the blood flow to the infant's brain during delivery, for example, due to inadequate circulation or perfusion, impaired respiratory effort, or inadequate ventilation.

Neonatal asphyxia can cause hypoxic damage to most of the infant's organs (heart, lungs, liver, gut, kidneys), but brain damage is of most concern and perhaps the least likely to quickly or completely heal. In more pronounced cases, an infant will survive, but with damage to the brain manifested as either mental, such as developmental delay or intellectual disability, or physical, such as spasticity. An infant suffering severe perinatal asphyxia usually has poor colour (cyanosis), perfusion, responsiveness, muscle tone, and respiratory effort. Extreme degrees of asphyxia can cause cardiac arrest and death. Neonatal asphyxia occurs in 2 to 10 per 1000 newborns that are born at term, and in higher instances for those that are born prematurely. WHO estimates that 4 million neonatal deaths occur yearly due to birth asphyxia, representing 38% of deaths of children under 5 years of age.

In one preferred embodiment, the combination or a pharmaceutical composition or a pharmaceutical product as described herein is formulated for intravenous administration.

In one preferred embodiment, the combination or a pharmaceutical composition or a pharmaceutical product as described herein is formulated for subcutaneous administration.

In one preferred embodiment, the combination or a pharmaceutical composition or a pharmaceutical product as described herein is formulated for oral administration.

In one preferred embodiment, the combination or a pharmaceutical composition or a pharmaceutical product as described herein is formulated for intranasal administration.

Another aspect relates to the use of:
(a) a first component which is a Nurr1 agonist; and
(b) at least one additional component selected from:
   (i) an aldosterone antagonist;
   (ii) an insulin modulator, and
   (iii) a sulfonylurea;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

One preferred embodiment of the invention relates to the use of:
amodiaquine, or a pharmaceutically acceptable salt thereof;
potassium canrenoate, or a structural or functional analogue thereof; and exenatide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

Another preferred embodiment of the invention relates to the use of:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

Another preferred embodiment of the invention relates to the use of:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection Another preferred embodiment of the invention relates to the use of:
amodiaquine, or a pharmaceutically acceptable salt thereof;
potassium canrenoate, or a structural or functional analogue thereof; and exenatide, or a structural or functional analogue thereof, or a pharmaceutically acceptable salt thereof; and
glibenclamide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

Another preferred embodiment of the invention relates to the use of:
amodiaquine, or a pharmaceutically acceptable salt thereof; and
exenatide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

Another aspect relates to a method of treating and/or preventing one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection, said method comprising simultaneously, sequentially or separately administering to a subject in need thereof:
(a) a first component which is a Nurr1 agonist; and
(b) at least one additional component selected from:
   (i) an aldosterone antagonist;
   (ii) an insulin modulator, and
   (iii) a sulfonylurea.

Preferred embodiments of the invention described above apply mutatis mutandis

In one embodiment, the subject is a mammal, more preferably a human.

In one preferred embodiment, the method comprises simultaneously, sequentially or separately administering to a subject in need thereof:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

In one preferred embodiment, the method comprises simultaneously, sequentially or separately administering to a subject in need thereof:
amodiaquine, or a pharmaceutically acceptable salt thereof;
potassium canrenoate, or a structural or functional analogue thereof; and
glibenclamide, or a structural or functional analogue thereof or a pharmaceutically acceptable salt thereof.

In one preferred embodiment, the method comprises simultaneously, sequentially or separately administering to a subject in need thereof:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

In one preferred embodiment, the method comprises simultaneously, sequentially or separately administering to a subject in need thereof:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof; and
glibenclamide, or a structural or functional analogue thereof, or a pharmaceutically acceptable salt thereof.

In one preferred embodiment, the method comprises simultaneously, sequentially or separately administering to a subject in need thereof:
amodiaquine, or a pharmaceutically acceptable salt thereof; and
exenatide, or a structural or functional analogue thereof, or a pharmaceutically acceptable salt thereof.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerine, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

The pharmaceutically active components of the combination can be administered separately or as a combined formulation. Preferably, the pharmaceutically active components are administered separately.

Each component can be administered by the same or different route to the other components.

In another preferred embodiment, the components of the combination are administered by the same route.

In another preferred embodiment, the components of the combination are administered by more than one route. For example, in one preferred embodiment, the insulin modulator is administered subcutaneously, and the other components of the combination are administered orally.

In one preferred embodiment, the components are administered parenterally (e.g., intravenously, intramuscularly, intradermally, intraperitoneally or subcutaneously).

In one preferred embodiment, the components are administered intravenously, intramuscularly, or subcutaneously. More preferably, the components are administered intravenously.

In another preferred embodiment, the components are administered orally.

In one preferred embodiment, the components are administered subcutaneously.

In another preferred embodiment, the components are administered intranasally.

In one preferred embodiment, the components are administered once a day.

In another preferred embodiment, the components are administered twice a day.

In one preferred embodiment, the components are administered by more than one route at different stages of the treatment.

In one preferred embodiment, the components are administered in a first administration phase and at least a second administration phase.

Within the first administration phase, the components of the combination can be administered by the same or different routes of administration.

Within the second administration phase, the components of the combination can be administered by the same or different routes of administration.

In one preferred embodiment, during the first administration phase, the components are administered parenterally, more preferably intravenously. Typically the first administration phase takes place in a clinical environment, e.g. a hospital or clinic.

During the second administration phase, the components of the combination can be administered by the same or a different route to the first administration phase.

In one preferred embodiment, during the second administration phase, the components of the combination are administered by a different route to the first administration phase, for example, orally, intranasally or subcutaneously, more preferably orally. This second administration phase can be termed a chronic administration phase and may last for an extended period, e.g. multiple days, weeks or months.

In one preferred embodiment, the components are administered in a first administration phase and at least one second administration phase, wherein the components in said first administration phase are administered parenterally, and the components in said at least one second administration phase are administered orally.

In one preferred embodiment, the components are administered in a first administration phase and at least one second administration phase, wherein the components in said first administration phase are administered intravenously, and the components in said at least one second administration phase are administered orally.

In one preferred embodiment, the components are administered in a first administration phase and at least one second administration phase, wherein the components in said first administration phase are administered parenterally, and the components in said at least one second administration phase are administered intranasally.

In one preferred embodiment, the components are administered in a first administration phase and at least one second administration phase, wherein the components in said first administration phase are administered intravenously, and the components in said at least one second administration phase are administered intranasally.

In one preferred embodiment, the components are administered in a first administration phase and at least one second administration phase, wherein the components in said first administration phase are administered parenterally, and the components in said at least one second administration phase are administered subcutaneously.

In one preferred embodiment, the components are administered in a first administration phase and at least one second administration phase, wherein the components in said first administration phase are administered intravenously, and the components in said at least one second administration phase are administered subcutaneously.

In one preferred embodiment, the components of the combination are administrated by different routes in the second administration phase. For example, in one preferred embodiment, the insulin modulator is administered subcutaneously and the other components of the combination are administered orally.

In one preferred embodiment, the first administration phase comprises administering 1 or 2 doses of the combination per day for a period of from about 1 to about 7 days, more preferably from about 2 to about 7 days.

In one preferred embodiment, in the first administration phase:
the Nurr1 agonist is administered in a dosage of from 0.05 to 0.5 mg/kg;
the insulin modulator is administered in a dosage of from 0.01 to 0.1 μg/kg;
the aldosterone antagonist is administered in a dosage of from 0.1 to 1 mg/kg;
the sulfonylurea is administered in a dosage of from 0.5 to 5 μg/kg.

Preferably, in the first administration phase the above dosages for the Nurr1 agonist, insulin modulator, aldosterone antagonist and sulfonylurea are administered once or twice a day, for a period of from about 1 to about 7 days, more preferably for a period of from about 2 to about 7 days.

In one preferred embodiment, the second administration phase comprises administering 1 or 2 doses per day for a period of from about 7 to about 90 days after the end of the first administration phase.

In one preferred embodiment, the second administration phase begins immediately after the end of the first administration phase. For example, in one preferred embodiment, the first administration phase ends on one day, and the second administration phase begins on the following day.

In another preferred embodiment, the first and second administration phases are separated by a time period during which none of the components of the inventive combination is administered, i.e. there is a delay between the first and second administration phases. For example, this time period (or delay) can be at least 1, 2, 3, 4,5,6,7, 14, or 21 days.

In one preferred embodiment, the invention comprises a first administration phase comprising 1 or 2 doses per day of a first dosage of the combination for a period of about 1 to about 7 days, more preferably 2 to about 7 days, followed by a second administration phase comprising 1 or 2 doses per day of a second dosage of the combination for a period of 7 to 90 days. Preferably, the first and second dosages are different.

Preferably, the second dosage is lower than the first dosage, i.e. the second administration phase can be considered a “chronic” administration phase. As used herein, “first dosage” and “second dosage” refer in each case to the dosages of the respective components of the combination. Without wishing to be bound by theory, it is believed that administering a higher dosage during a first administration phase can be advantageous in mitigating the harmful effects of reperfusion injury, whereas administering a lower dosage in a second administration phase, for example, over a more prolonged period of time, can be potentially beneficial to the immune response after stroke, thereby improving the restoration of brain function.

In another preferred embodiment, the second administration phase comprises 1 or 2 doses per day of a second dosage of the combination administered for a period of at least 30 days after the end of the first administration phase. In another preferred embodiment, the second administration phase comprises 1 or 2 doses per day of a second dosage of the combination administered for a period of at least 60 days after the end of the first administration phase. In another preferred embodiment, the second administration phase comprises 1 or 2 doses per day of a second dosage of the combination administered for a period of at least 90 days after the end of the first administration phase.

In one preferred embodiment, in the second administration phase:
the Nurr1 agonist is administered in a dosage of from 0.05 to 0.5 mg/kg;
the insulin modulator is administered in a dosage of from 0.01 to 0.1 μg/kg;
the aldosterone antagonist is administered in a dosage of from 0.1 to 1 mg/kg;
the sulfonylurea is administered in a dosage of from 0.5 to 5 μg/kg.

Preferably, in the second administration phase the above doses for the Nurr1 agonist, insulin modulator, aldosterone antagonist and sulfonylurea are administered once or twice a day, for a period of 7 to 90 days after the end of the first administration phase.

In one preferred embodiment, the method comprises simultaneously administering the components to said subject.

In one preferred embodiment, each of the pharmaceutically active components of the combination, pharmaceutical product or pharmaceutical composition is administered separately.

The components of the inventive combination may be for administration simultaneously, sequentially or separately (as part of a dosing regimen).

Amodiaquine, exenatide or structural or functional analogues thereof or pharmaceutically acceptable salts thereof, potassium canrenoate or structural or functional analogues thereof, and glibenclamide or structural or functional analogues or pharmaceutically acceptable salts thereof, may be for administration simultaneously, sequentially or separately (as part of a dosing regimen).

As used herein, “simultaneously” is used to mean that the two agents are administered concurrently.

As used herein, “sequentially” is used to mean that the active agents are not administered concurrently, but one after the other. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction there between, and their respective half-lives.

In contrast to “sequentially”, “separately” is used herein to mean that the gap between administering one agent and the other is significant i.e. the first administered agent may no longer be present in the bloodstream in a therapeutically effective amount when the second agent is administered.

In one embodiment, the components are administered simultaneously.

In one embodiment, the components are administered sequentially or separately.

For a two component combination, the two components can be administered simultaneously or separately, in any order.

For a three component combination, all three components can be administered simultaneously, or any two components can be administered simultaneously, with the third component administered separately or sequentially. Alternatively, all three components can be administered in any order separately or sequentially.

For a four component combination, all four components can be administered simultaneously, or two or three components can be administered simultaneously, with the remaining component(s) administered separately or sequentially. Alternatively, all four components can be administered in any order separately or sequentially.

In one embodiment, the components are each administered in a therapeutically effective amount with respect to the individual components.

As used herein, the term “therapeutically effective amount” refers to a quantity sufficient to achieve a desired therapeutic and/or prophylactic effect, e.g., an amount which results in the prevention of, or a decrease in, ischemia and/or reperfusion injury or one or more symptoms associated with ischemia and/or reperfusion injury.

In the context of therapeutic or prophylactic applications, the amount of a composition administered to the subject will depend on the type and severity of the disease and on the characteristics of the individual, such as general health, age, sex, body, weight and tolerance to drugs. It will also depend on the degree severity and type of disease. The skilled artisan will be able to determine appropriate dosages depending on these and other factors. The composition can also be administered in combination with one or more additional therapeutic agents.

In one embodiment, the components are each administered in a sub-therapeutically effective amount with respect to the individual components.

In one embodiment, the components are administered prior to reperfusion of the subject.

In one embodiment, the components are administered during reperfusion of the subject.

In one embodiment, the components are administered after reperfusion of the subject.

In one embodiment, the components are administered prior to and/or during and/or after reperfusion of the subject.

In some embodiments, one or more of the components are administered continuously before, during, and after reperfusion of the subject, and the remaining components are administered prior to reperfusion or after reperfusion.

In some embodiments, the subject is administered the components continuously before, during, and after reperfusion of the subject.

In some embodiments, additional administration of one or more of the components may occur after reperfusion. Preferably, this repeat administration is carried out at least twice, more preferably from 2 to 100 times, or can be in the form of continuous infusion.

In some embodiments of the method, the subject is administered the components as a bolus dose prior to reperfusion.

In some embodiments of the method, the subject is administered the components as a bolus dose during reperfusion.

In some embodiments of the method, the subject is administered the components as a bolus dose after reperfusion.

As used herein “reperfusion” is the restoration of blood flow to any organ or tissue in which the flow of blood is decreased or blocked. For example, blood flow can be restored to any organ or tissue affected by ischemia or hypoxia. The restoration of blood flow (reperfusion) can occur by any method known to those in the art. For instance, reperfusion of ischemic cardiac tissues may arise from revascularization.

In one embodiment, reperfusion is achieved via a revascularization procedure. In one embodiment, the revascularization procedure is selected from the group consisting of: percutaneous coronary intervention; balloon angioplasty; insertion of a bypass graft; insertion of a stent; directional coronary atherectomy; treatment with a one or more thrombolytic agent(s); and removal of an occlusion.

In one embodiment, the one or more thrombolytic agents are selected from the group consisting of: tissue plasminogen activator; urokinase; prourokinase; streptokinase; acylated form of plasminogen; acylated form of plasmin; and acylated streptokinase-plasminogen complex.

### Dosage

A person of ordinary skill in the art can easily determine an appropriate dose of one of the instant compositions to administer to a subject without undue experimentation.

Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In one highly preferred embodiment of the invention, the dose of the Nurr1 agonist (e.g. amodiaquine) in the combination is generally lower than the dose typically used in monotherapy in the context of its currently approved therapies, and/or lower than the general doses reported in the reperfusion injury literature.

In one highly preferred embodiment of the invention, the dose of the insulin modulator (e.g. exenatide) in the combination is generally lower than the dose typically used in monotherapy in the context of its currently approved therapies, and/or lower than the general doses reported in the reperfusion injury literature.

In one highly preferred embodiment of the invention, the dose of the aldosterone antagonist (e.g. potassium canrenoate) in the combination is generally lower than the dose typically used in monotherapy in the context of its currently approved therapies, and/or lower than the general doses reported in the reperfusion injury literature.

In one highly preferred embodiment of the invention, the dose of the sulfonylurea (e.g. glibenclamide) in the combination is generally lower than the dose typically used in monotherapy in the context of its currently approved therapies, and/or lower than the general doses reported in the reperfusion injury literature.

Each component of the claimed combination may be formulated in unit dosage form, i.e., in the form of discrete portions containing a unit dose, or a multiple or sub-unit of a unit dose. The dosages described herein are applicable to each of the above-described medical uses.

In one preferred embodiment, the amodiaquine, or pharmaceutically acceptable salt thereof, is administered at a dosage of about 0.01 to about 20 mg/kg body weight of the subject, preferably about 0.1 to about 10 mg/kg, more preferably about 0.1 to about 5 mg/kg, even more preferably about 0.1 to about 1 mg/kg. In another preferred embodiment, the amodiaquine, or pharmaceutically acceptable salt thereof, is administered at a dosage of about 1 to about 10 mg/kg or about 1 to about 5 mg/kg. In one preferred embodiment, the amodiaquine, or pharmaceutically acceptable salt thereof, is administered at a dosage of about 2 mg/kg or about 7 mg/kg. In one particularly preferred embodiment, the amodiaquine, or pharmaceutically acceptable salt thereof, is administered at a dosage of about 0.1 to about 0.5 mg/kg, more preferably about 0.1 to about 0.25 mg/kg. Advantageously, the dosages of amodiaquine suitable for use in the present combinations are significantly lower than reported in the literature, for example, 40 mg/kg in the context of treating intracerebral haemorrhage (Kinoshita et al. 2019), and 20 mg/kg in the context of enhancing cognitive function by increasing adult hippocampal neurogenesis (Kim et al. 2016).

In one highly preferred embodiment, the amodiaquine, or pharmaceutically acceptable salt thereof, is administered at a dosage of about 0.5 mg/kg. In one highly preferred embodiment, the amodiaquine, or pharmaceutically acceptable salt thereof, is administered at a dosage of about 0.1 mg/kg or about 0.15 mg/kg.

When used in the presently claimed combinations, the insulin modulator (e.g. exenatide) is preferably administered in a dose of from about 0.001 to about 1.5 μg/kg, more preferably from about 0.005 to about 0.15 μg/kg. In one preferred embodiment, the insulin modulator (e.g. exenatide) is preferably administered in a dose of from about 0.01 to about 1.5 μg/kg, more preferably from about 0.05 to about 1.5 μg/kg. As used herein the insulin modulator dosages are μg/kg body weight (μg=microgram).

In one preferred embodiment, the insulin modulator (e.g. exenatide) is preferably administered in a dose of from about 0.01 to about 0.5 μg/kg, more preferably from about 0.02 to about 0.5 μg/kg, or from about 0.03 to about 0.5 μg/kg, or from about 0.04 to about 0.5 μg/kg, or from about 0.05 to about 0.5 μg/kg, or from about 0.05 to about 0.2 μg/kg, or from about 0.05 to about 0.15 μg/kg.

In one preferred embodiment, the insulin modulator (e.g. exenatide) is preferably administered in a dose of from about 0.01 to about 0.1 μg/kg, more preferably from about 0.02 to about 0.08 μg/kg, or from about 0.03 to about 0.07 μg/kg, or from about 0.04 to about 0.06 μg/kg, or in a dose of about 0.05 μg/kg.

When used in the presently claimed combinations, the aldosterone antagonist (e.g. potassium canrenoate) is preferably administered in a dose of from about 0.03 to about 10 mg/kg, or about 0.1 to about 10 mg/kg or about 0.3 to about 5 mg/kg, or from about 1 to about 10 mg/kg, or from about 1 to about 5 mg/kg, or from about 1 to about 3 mg/kg. As used herein the aldosterone antagonist dosages are in mg/kg body weight.

In one preferred embodiment, the aldosterone antagonist (e.g. potassium canrenoate) is preferably administered in a dose of from about 0.1 to about 3 mg/kg or from about 0.2 to about 2 mg/kg, or from about 0.3 to about 1.5 mg/kg, or from about 0.3 to about 1 mg/kg.

In one preferred embodiment, the aldosterone antagonist (e.g. potassium canrenoate) is preferably administered in a dose of from about 0.1 to about 0.5 mg/kg or from about 0.2 to about 0.5 mg/kg, more preferably, from about 0.2 to about 0.4 mg/kg, even more preferably, about 0.3 to 0.4 mg/kg.

When used in the presently claimed combinations, the sulfonylurea (e.g. glibenclamide) is preferably administered in a dose of from about 0.001 to about 30 μg/kg, more preferably from about 0.01 to about 5 μg/kg, even more preferably from about 0.01 to about 2 μg/kg. As used herein the sulfonylurea dosages are in μg/kg body weight.

In one preferred embodiment, the sulfonylurea (e.g. glibenclamide) is preferably administered in a dose of from about 0.5 to about 20 μg/kg, or from about 0.5 to about 15 μg/kg, or from about 0.5 to about 10 μg/kg, or from about 1 to about 10 μg/kg.

In one preferred embodiment, the sulfonylurea (e.g. glibenclamide) is preferably administered in a dose of from about 0.5 to about 8 μg/kg, or from about 0.5 to about 7 μg/kg, or from about 0.5 to about 6 μg/kg, or from about 0.5 to about 5 μg/kg. In one preferred embodiment, the sulfonylurea (e.g. glibenclamide) is preferably administered in a dose of from about 0.5 to about 3 μg/kg, or from about 0.5 to about 2 μg/kg, or from about 0.5 to about 1.5 μg/kg, or from about 0.8 to about 1.2 μg/kg, or at about 1 μg/kg.

In one highly preferred embodiment, the combination is a fixed dose combination comprising predetermined dosages of the respective pharmaceutically active components e.g. to allow administration to the subject of the above described dosages, for example, about 0.001 to about 1.5 μg/kg exenatide, from about 0.001 to about 30 μg/kg glibenclamide, from about 0.03 to about 10 mg/kg potassium canrenoate, and from about 0.01 to about 20 mg/kg amodiaquine.

Preferably, the fixed dose combination comprises predetermined dosages of the respective pharmaceutically active components to allow administration to the subject of the following doses.

In one highly preferred embodiment, the combination is a fixed dose combination comprising about 0.01 to about 0.5 μg/kg exenatide and from about 0.01 to about 20 mg/kg amodiaquine.

In one highly preferred embodiment, the combination is a fixed dose combination comprising about 0.01 to about 0.1 μg/kg exenatide and from about 0.1 to about 10 mg/kg amodiaquine.

In one highly preferred embodiment, the combination is a fixed dose combination comprising about 0.01 to about 0.1 μg/kg exenatide and from 0.1 to about 5 mg/kg amodiaquine.

In one highly preferred embodiment, the combination is a fixed dose combination comprising about 0.01 to about 0.1 μg/kg exenatide and from about 0.1 to about 1 mg/kg amodiaquine, more preferably, from about 0.1 to about 0.5 mg/kg amodiaquine.

In one highly preferred embodiment, the combination is a fixed dose combination comprising predetermined dosages of the respective components e.g. about 0.03 to about 10 mg/kg potassium canrenoate, from about 0.001 to about 30 μg/kg glibenclamide and from about 0.01 to about 20 mg/kg amodiaquine.

In one highly preferred embodiment, the combination is a fixed dose combination comprising about 0.1 to about 3 mg/kg potassium canrenoate, from about 0.5 to about 20 μg/kg glibenclamide, and from about 0.1 to about 10 mg/kg amodiaquine.

In one highly preferred embodiment, the combination is a fixed dose combination comprising about 0.1 to about 0.5 mg/kg potassium canrenoate, from about 0.5 to about 8 μg/kg glibenclamide, from about 0.1 to about 5 mg/kg amodiaquine.

In one highly preferred embodiment, the combination is a fixed dose combination comprising about 0.1 to about 0.5 mg/kg potassium canrenoate, from about 0.5 to about 1.5 μg/kg glibenclamide, and from 0.1 to about 1 mg/kg amodiaquine, more preferably, from about 0.1 to about 0.5 mg/kg amodiaquine.

In one highly preferred embodiment, the combination is a fixed dose combination comprising predetermined dosages of the respective components e.g. about 0.03 to about 10 mg/kg potassium canrenoate, from about 0.005 to about 0.15 μg/kg exenatide and from about 0.01 to about 20 mg/kg amodiaquine.

In one highly preferred embodiment, the combination is a fixed dose combination comprising about 0.1 to about 3 mg/kg potassium canrenoate, from about 0.01 to about 0.5 μg/kg exenatide, and from about 0.1 to about 10 mg/kg amodiaquine.

In one highly preferred embodiment, the combination is a fixed dose combination comprising about 0.1 to about 0.5 mg/kg potassium canrenoate, from about 0.01 to about 0.1 μg/kg exenatide and from about 0.1 to about 5 mg/kg amodiaquine.

In one highly preferred embodiment, the combination is a fixed dose combination comprising about 0.1 to about 0.5 mg/kg potassium canrenoate, from about 0.01 to about 0.1 μg/kg exenatide, and from about 0.1 to about 1 mg/kg amodiaquine, more preferably, from about 0.1 to about 0.5 mg/kg amodiaquine.

In one highly preferred embodiment, the combination is a fixed dose combination comprising about 0.03 to about 10 mg/kg potassium canrenoate, from about 0.01 to about 0.5 μg/kg exenatide, from about 0.001 to about 30 μg/kg glibenclamide, and from 0.01 to about 20 mg/kg amodiaquine.

In one highly preferred embodiment, the combination is a fixed dose combination comprising about 0.1 to about 3 mg/kg potassium canrenoate, from about 0.01 to about 0.1 μg/kg exenatide, from about 0.5 to about 20 μg/kg glibenclamide, and from 0.1 to about 10 mg/kg amodiaquine.

In one highly preferred embodiment, the combination is a fixed dose combination comprising about 0.1 to about 0.5 mg/kg potassium canrenoate, from about 0.01 to about 0.1 μg/kg exenatide, from about 0.5 to about 8 μg/kg glibenclamide, and from 0.1 to about 5 mg/kg amodiaquine.

In one highly preferred embodiment, the combination is a fixed dose combination comprising about 0.1 to about 0.5 mg/kg potassium canrenoate, from about 0.01 to about 0.1 μg/kg exenatide, from about 0.5 to about 1.5 μg/kg glibenclamide, and from 0.1 to about 1 mg/kg amodiaquine, more preferably, from about 0.1 to about 0.5 mg/kg amodiaquine.

In one highly preferred embodiment, the combination is a fixed dose combination comprising about 0.05 μg/kg exenatide, 1 μg/kg glibenclamide, and about 0.5 mg/kg amodiaquine.

In one highly preferred embodiment, the combination is a fixed dose combination comprising about 0.33 mg/kg potassium canrenoate, about 1 μg/kg glibenclamide, and about 0.5 mg/kg amodiaquine.

In one highly preferred embodiment, the combination is a fixed dose combination comprising about 0.05 μg/kg exenatide, about 0.33 mg/kg potassium canrenoate, about 1 μg/kg glibenclamide and about 0.5 mg/kg amodiaquine.

In one highly preferred embodiment, the combination is a fixed dose combination comprising about 0.05 μg/kg exenatide, and about 0.5 mg/kg amodiaquine.

### Non-Therapeutic Use

In another aspect, the present invention relates to use of a combination comprising, or consisting of:
(a) a first component which is a Nurr1 agonist; and
(b) at least one additional component selected from:
   (i) an aldosterone antagonist;
   (ii) an insulin modulator, and
   (iii) a sulfonylurea;
for treating and/or preventing ischemia and/or reperfusion injury in an ex vivo organ prior to or during transplantation.

In one preferred embodiment, the invention relates to the use of a combination comprising, or consisting of:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof;
for treating and/or preventing ischemia and/or reperfusion injury in an ex vivo organ prior to or during transplantation.

In another preferred embodiment, the invention relates to the use of a combination comprising, or consisting of:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof;
for treating and/or preventing ischemia and/or reperfusion injury in an ex vivo organ prior to or during transplantation.

In another preferred embodiment, the invention relates to the use of a combination comprising, or consisting of:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof;
for treating and/or preventing ischemia and/or reperfusion injury in an ex vivo organ prior to or during transplantation.

In another preferred embodiment, the invention relates to the use of a combination comprising, or consisting of:
amodiaquine, or a pharmaceutically acceptable salt thereof;
potassium canrenoate, or a structural or functional analogue thereof;
exenatide, or a structural or functional analogue thereof, or a pharmaceutically acceptable salt thereof; and
glibenclamide, or a structural or functional analogue thereof, or a pharmaceutically acceptable salt thereof;
for treating and/or preventing ischemia and/or reperfusion injury in an ex vivo organ prior to or during transplantation.

In another preferred embodiment, the invention relates to the use of a combination comprising, or consisting of:
amodiaquine, or a pharmaceutically acceptable salt thereof; and
exenatide, or a structural or functional analogue thereof, or a pharmaceutically acceptable salt thereof;
for treating and/or preventing ischemia and/or reperfusion injury in an ex vivo organ prior to or during transplantation.

An ex vivo (removed from the body) organ can be susceptible to reperfusion injury due to lack of blood flow. Therefore, the combination of the present invention can be used to prevent reperfusion injury in the removed organ. Preferably, the organ is a heart, liver or kidney, more preferably, a heart.

In some embodiments, the removed organ is placed in a standard buffered solution, such as those commonly used in the art, containing the combination of the invention.

For example, a removed heart can be placed in a cardioplegic solution containing amodiaquine, and one or more of exenatide, potassium canrenoate and glibenclamide.

The concentration of amodiaquine, exenatide, potassium canrenoate and glibenclamide useful in the standard buffered solution can be easily determined by those skilled in the art. Such concentrations may be, for example, between about 0.1 nM to about 10 μM, preferably about 1 nM to about 10 μM.

The invention is further described with reference to the accompanying non-limiting examples, and the following figures wherein:

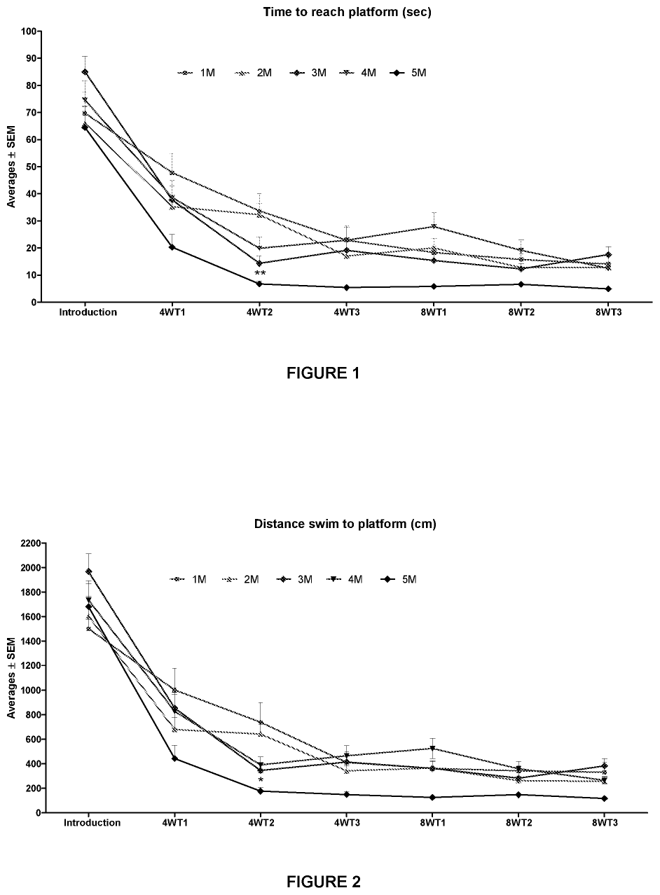

FIG. 1: Time to find the hidden platform (in see; Average±SEM) on Week 4 and Week 8 three trials T1, T2 and T3 on each test. See Example 1. Statistical analysis —**p<0.01 according to two-way ANOVA followed by Bonferroni post-hoc comparisons (3M against 1M). Group 1M: Vehicle treated controls; Group 2M: Exenatide 0.05 μg/kg+Potassium canrenoate 0.33 mg/kg+Glibenclamide 1 μg/kg; Group 3M: Exenatide 0.05 μg/kg+Potassium canrenoate 0.33 mg/kg+Glibenclamide 1 μg/kg+Amodiaquine 20 mg/kg; Group 4M: Exenatide 0.05 μg/kg+Potassium canrenoate 0.33 mg/kg+Amodiaquine 20 mg/kg; Group 5M: Sham operated.

FIG. 2: Distance swam to reach the hidden platform (in cm; Average±SEM on Week 4 and Week 8 three trials T1, T2 and T3 on each test). See Example 1. Statistical analysis—*p<0.05 according to two-way ANOVA followed by Bonferroni post-hoc comparisons (3M against 1M). Group 1M: Vehicle treated controls; Group 2M: Exenatide 0.05 μg/kg+Potassium canrenoate 0.33 mg/kg+Glibenclamide 1 μg/kg; Group 3M: Exenatide 0.05 μg/kg+Potassium canrenoate 0.33 mg/kg+Glibenclamide 1 μg/kg+Amodiaquine 20 mg/kg; Group 4M: Exenatide 0.05 μg/kg+Potassium canrenoate 0.33 mg/kg+Amodiaquine 20 mg/kg; Group 5M: Sham operated.

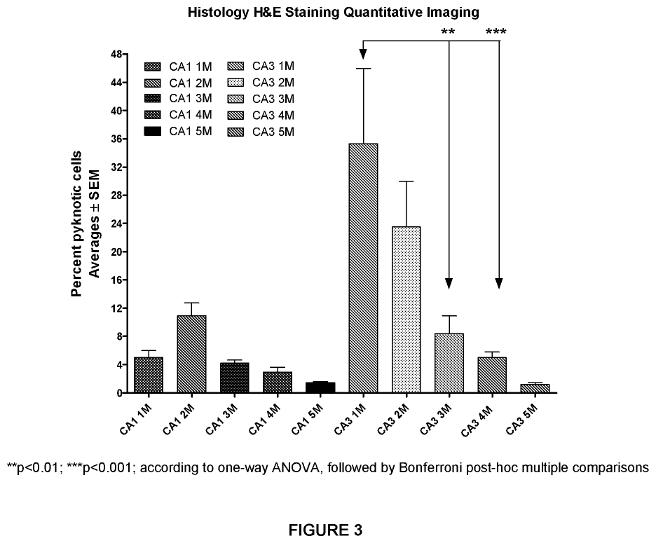

FIG. 3: Hippocampal CA1 and CA3 cells damage upon study termination. See Example 1. Group 1M: Vehicle treated controls; Group 2M: Exenatide 0.05 μg/kg+Potassium canrenoate 0.33 mg/kg+Glibenclamide 1 μg/kg; Group 3M: Exenatide 0.05 μg/kg+Potassium canrenoate 0.33 mg/kg+Glibenclamide 1 μg/kg+Amodiaquine 20 mg/kg; Group 4M: Exenatide 0.05 μg/kg+Potassium canrenoate 0.33 mg/kg+Amodiaquine 20 mg/kg; Group 5M: Sham operated.

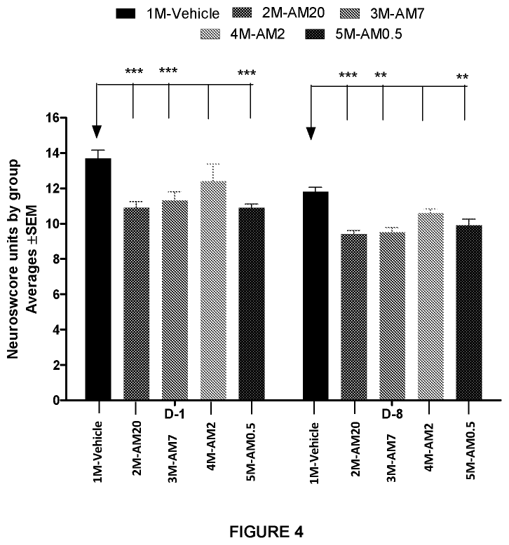

FIG. 4: Neurological scores throughout the study (Amodiaquine monotherapy) (Part 1). See Example 2. AM20=Amodiaquine 20 mg/kg; AM7=Amodiaquine 7 mg/kg; AM2=Amodiaquine 2 mg/kg; AM0.5=Amodiaquine 0.5 mg/kg. Before surgery all animals behaved normally. Statistically significant differences, according to two-way ANOVA followed by Bonferroni post-hoc comparisons, were found between Group 1M (Vehicle) and the drug treated groups 2M Amodiaquine (20 mg/kg), 3M Amodiaquine (7 mg/kg) and 5M Amodiaquine (0.5 mg/kg) on Day 1 and on Day 8. The figure shows averages±SEM; ** indicates p<0.01, *** indicates p<0.001.

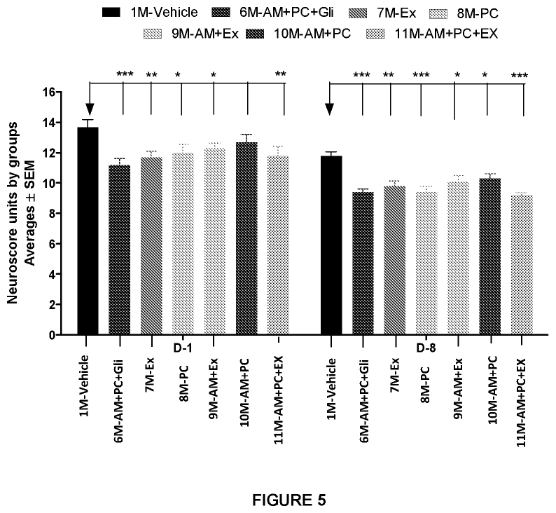

FIG. 5: Neurological scores throughout the study (Combined therapy) (Part II). See Example 2. AM=Amodiaquine; PC=Potassium Canrenoate; Gli=Glibenclamide; Ex=Exenatide. Before surgery all animals behaved normally. Statistically significant differences were found between Group 1M (Vehicle) and the drug treated groups 6M, 7M, 8M, 9M and 11M on Day 1 and for all treated groups on Day 8. The figure shows averages±SEM; * indicates p<0.05, ** indicates p<0.01, *** indicates p<0.001, using two-way ANOVA statistical analysis followed by Bonferroni post-hoc comparisons.

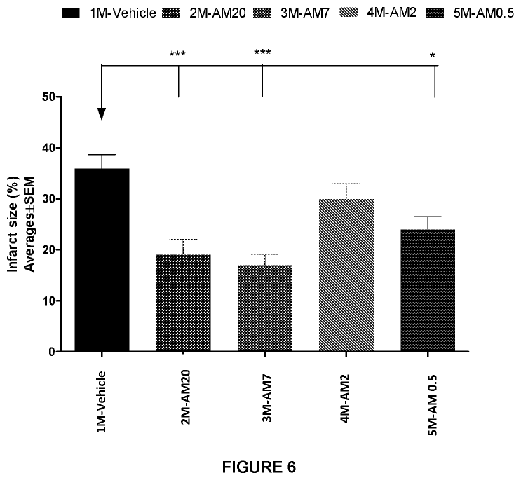

FIG. 6: Brain infarct size (%) at termination (Amodiaquine monotherapy) (Part 1). See Example 2. AM20=Amodiaquine 20 mg/kg; AM7=Amodiaquine 7 mg/kg; AM2=Amodiaquine 2 mg/kg; AM0.5=Amodiaquine 0.5 mg/kg. Statistically significant differences in infarct size were found between Group 1M (Vehicle) and the drug treated groups 2M Amodiaquine 20 mg/kg, 3M Amodiaquine 7 mg/kg and 5M Amodiaquine 0.5 mg/kg. The figure shows averages±SEM; * indicates p<0.05; *** indicates p<0.001 using One-way ANOVA statistical analysis followed by Bonferroni's multiple comparison test.

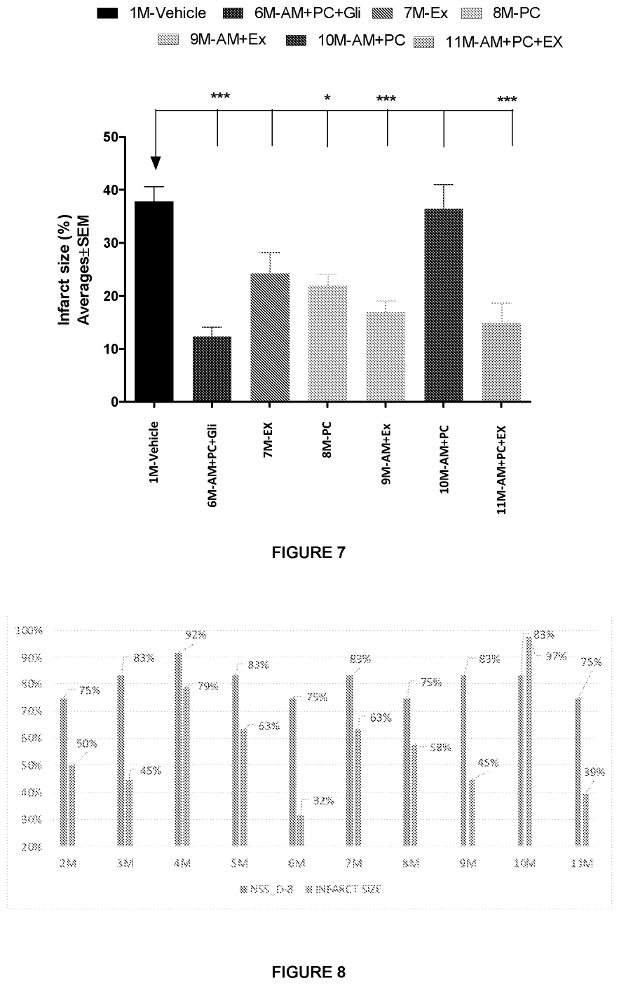

FIG. 7: Brain infarct size (%) at termination (Combined therapy) (Part II). See Example 2. AM=Amodiaquine; PC=Potassium Canrenoate; Gli=Glibenclamide; Ex=Exenatide. Statistically significant differences in infarct size were found between Group 1M (Vehicle) and the drug treated groups 6M, 8M, 9M and 11M. The figure shows Averages±SEM; * indicates p<0.05; *** indicates p<0.001 using one-way ANOVA statistical analysis followed by Bonferroni's multiple comparison test.

FIG. 8: Summary of NSS score and infarct size at Day 8 for Groups 2M-11M expressed as percentage of the control values. See Example 2.

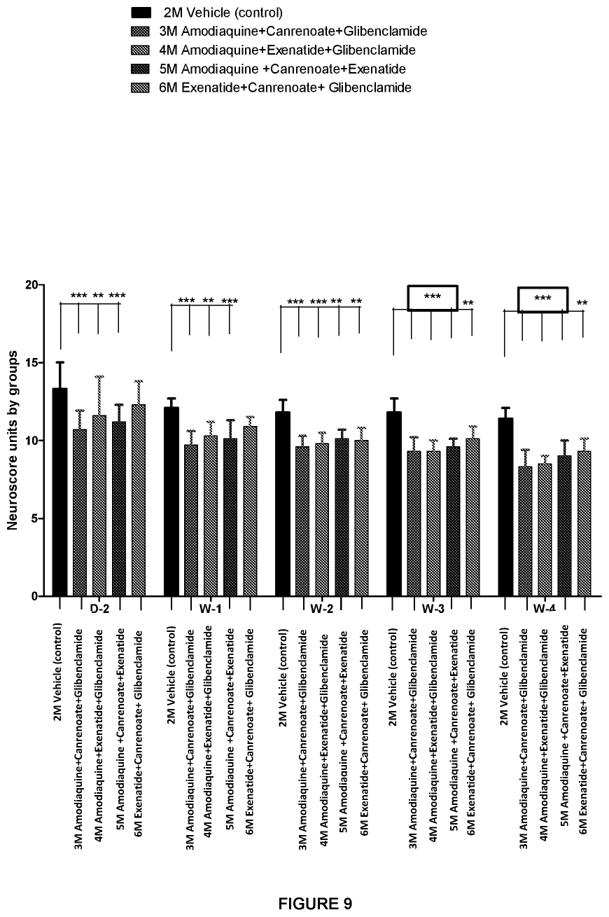

FIG. 9 shows the neurological severity score for selected combinations in a rat model of chronic stroke study as described in Example 3 (Geometric mean±SD). Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

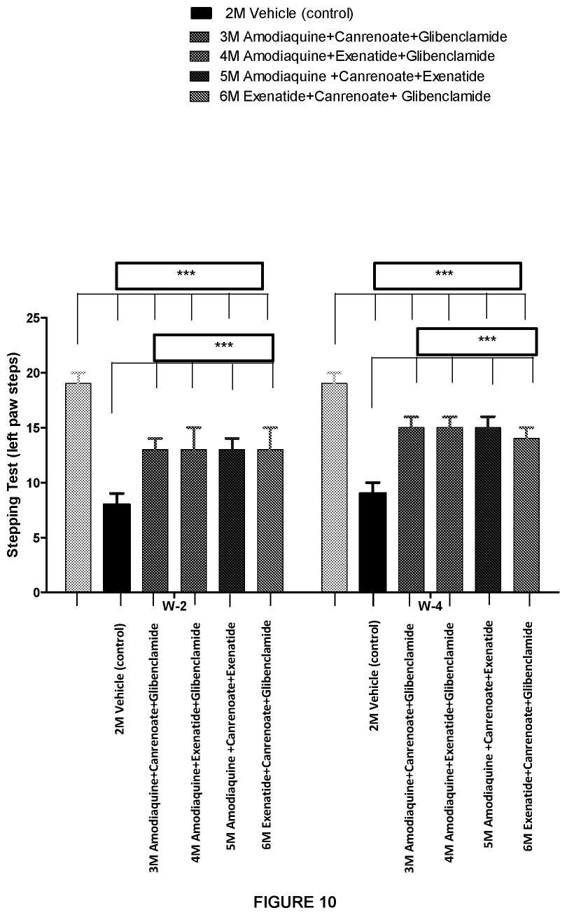

FIG. 10 shows the results of a stepping test for selected combinations in a rat model of chronic stroke study as described in Example 3 (Geometric mean±SD). Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

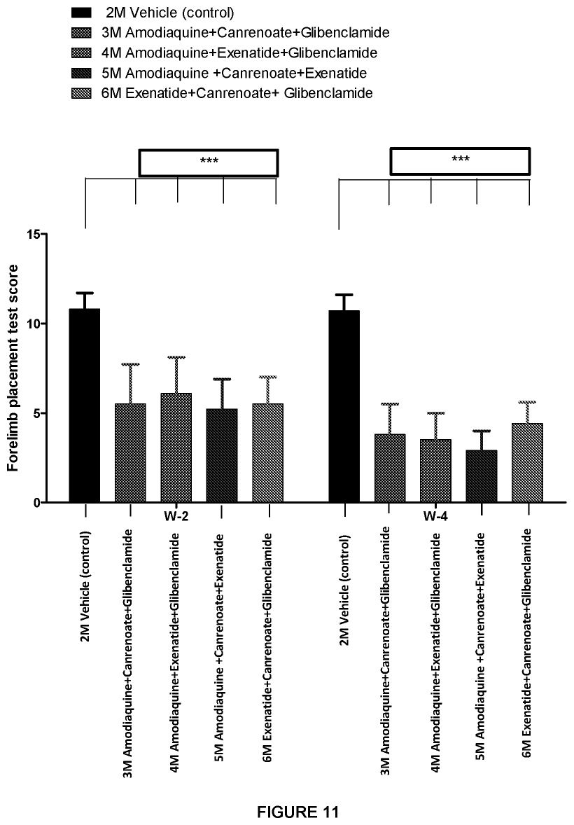

FIG. 11 shows the results of a forelimb placement test for selected combinations in a rat model of chronic stroke study as described in Example 3 (Geometric mean±SD). Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

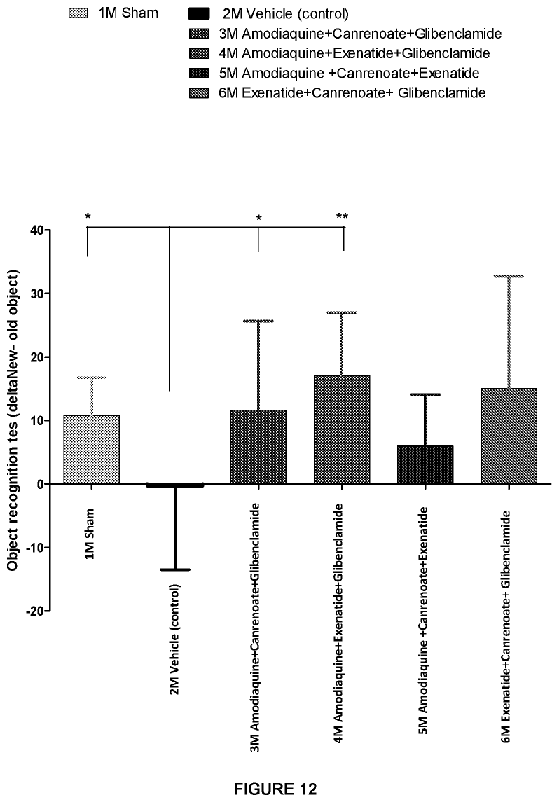

FIG. 12 shows the results of an object recognition test for selected combinations in a rat model of chronic stroke study as described in Example 3 (Geometric mean±SD). Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

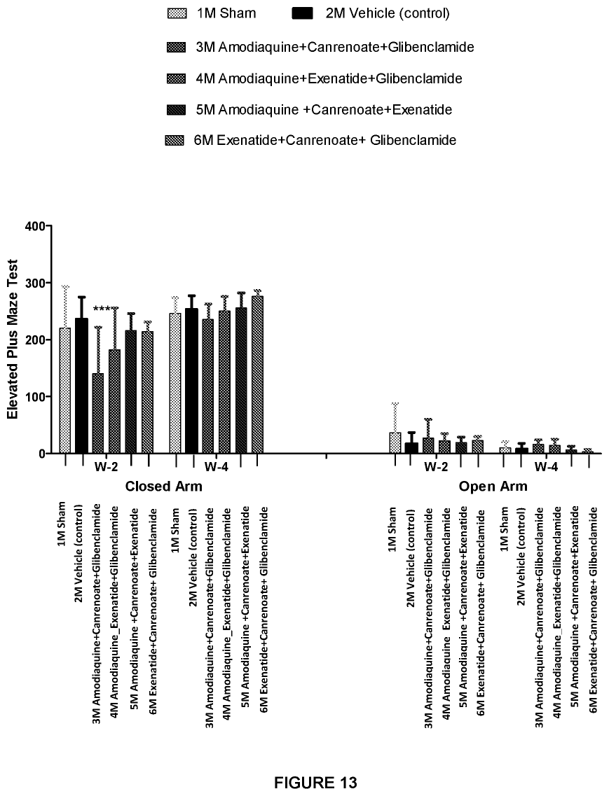

FIG. 13 shows the results of an elevated plus maze test for selected combinations in a rat model of chronic stroke study as described in Example 3 (Geometric mean±SD). Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

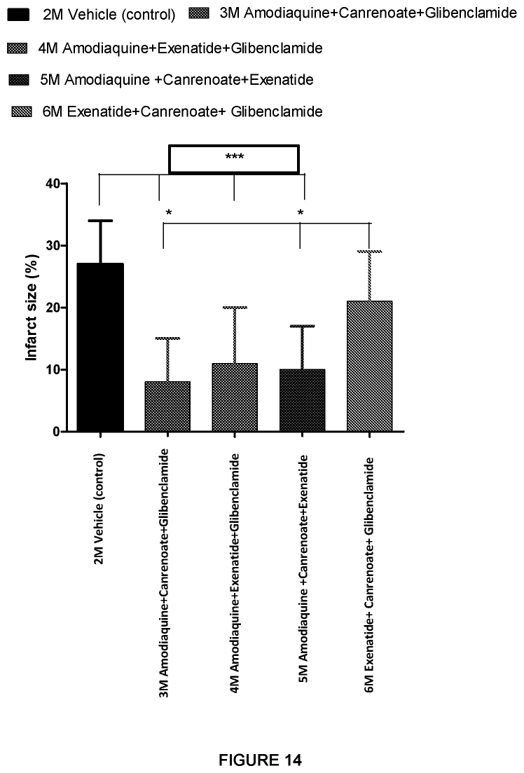

FIG. 14 shows infarct size percentage at 28 days for selected combinations in a rat model of chronic stroke study as described in Example 3. Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

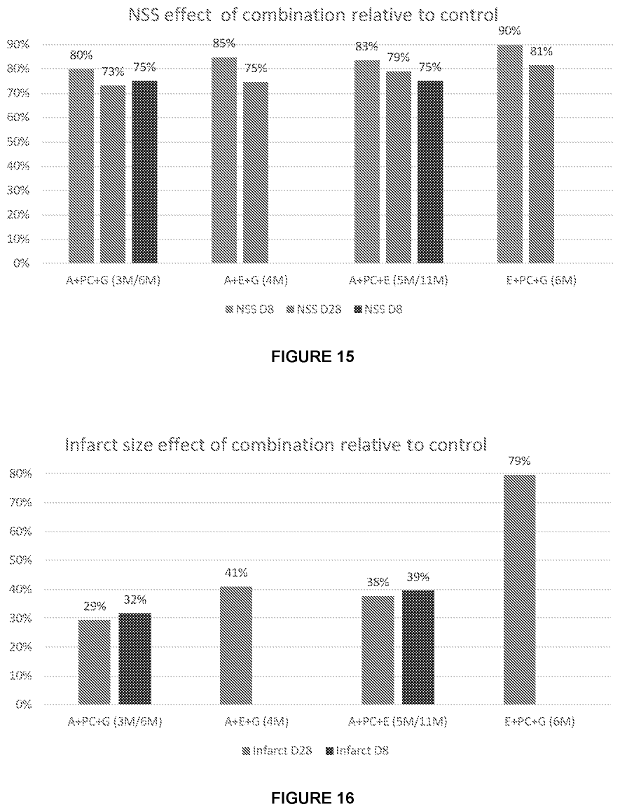

FIG. 15 shows the NSS effect of selected combinations relative to control (%) from Examples 2 and 3 (days 8 and 28).

FIG. 16 shows the infarct size effect of selected combinations relative to control (%) from Examples 2 and 3 (days 8 and 28).

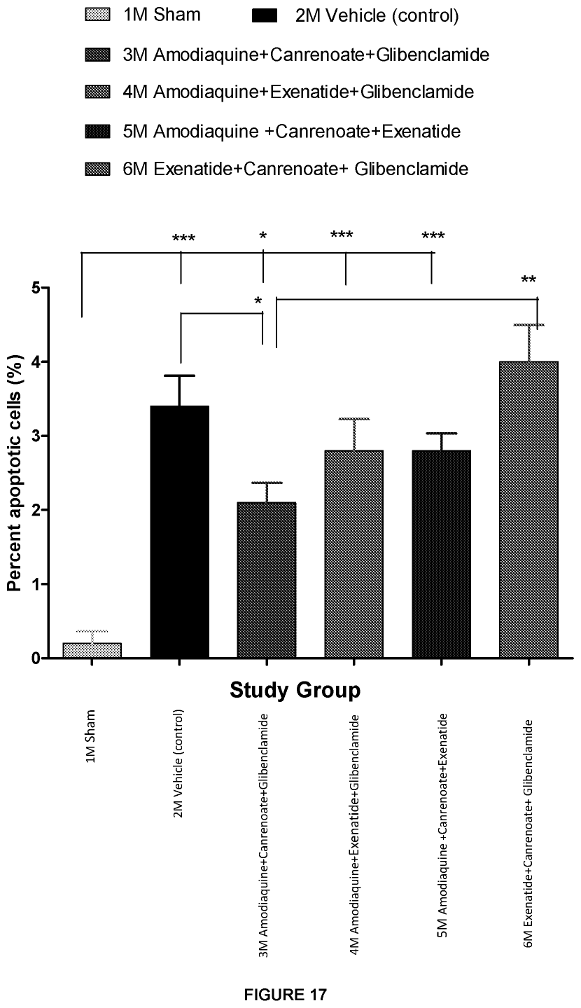

FIG. 17 shows apoptosis (TUNEL staining) in α-penumbra, striatum and dorsal hippocampus areas at study termination (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

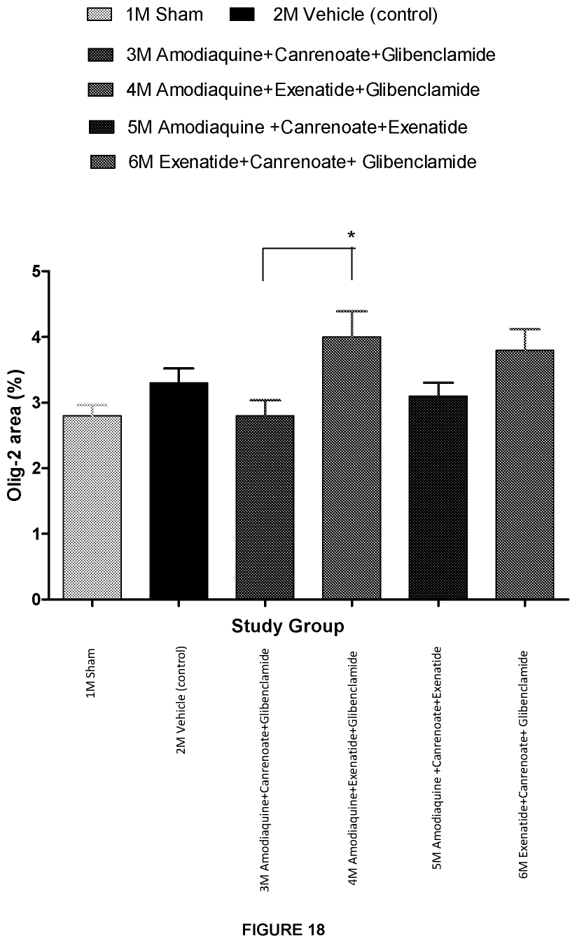

FIG. 18 shows Olig-2 mature oligodendrocytes—percent of activated cells at study termination (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

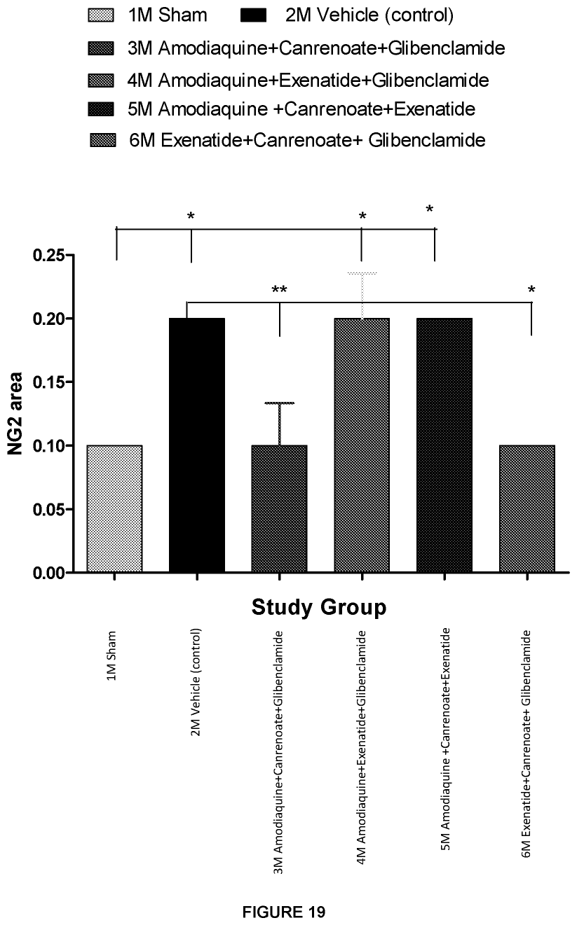

FIG. 19 shows NG2 oligodendrocyte progenitor cells—percent of cells at study termination (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

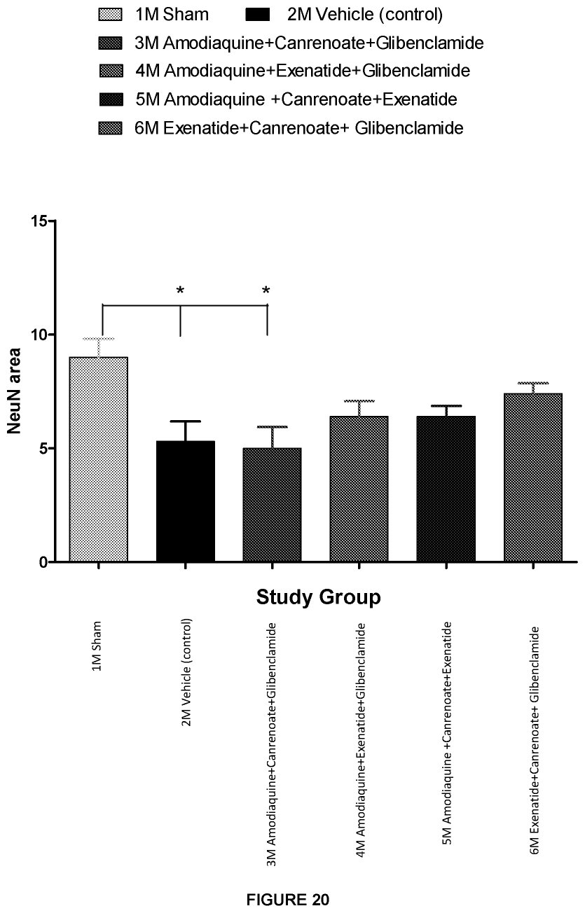

FIG. 20 shows NeuN damage at study termination (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

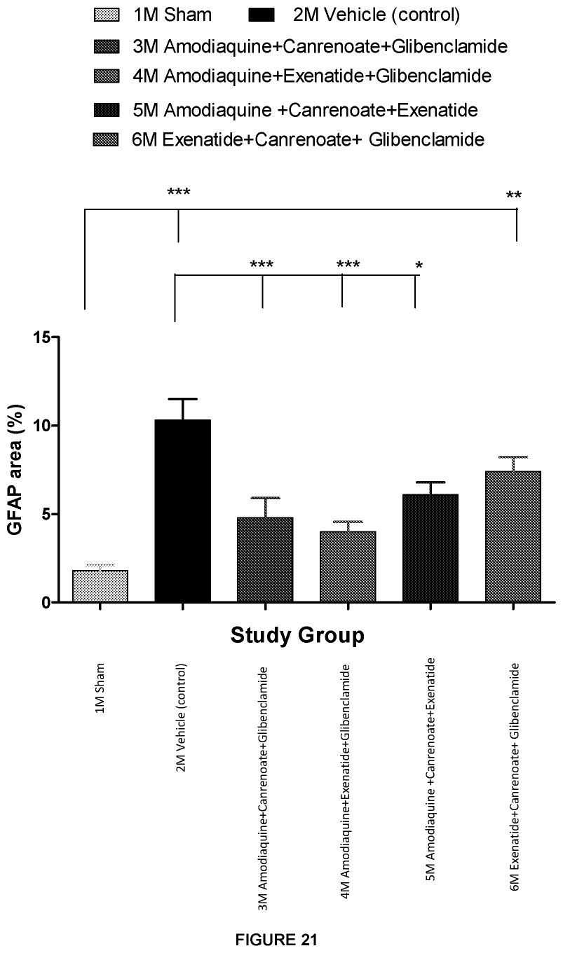

FIG. 21 shows GFAP histology results at study termination (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

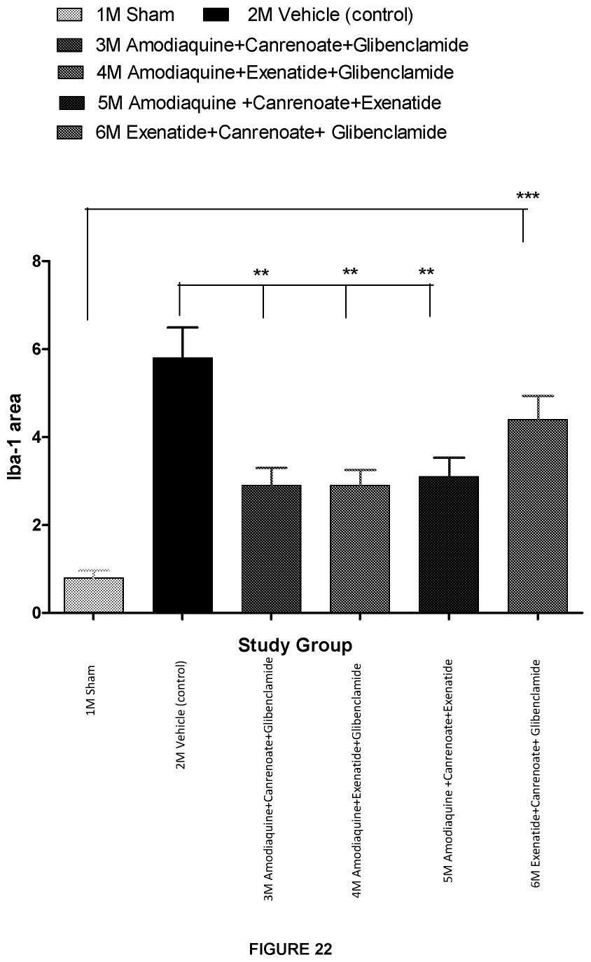

FIG. 22 shows Iba-1 histology results at study termination (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

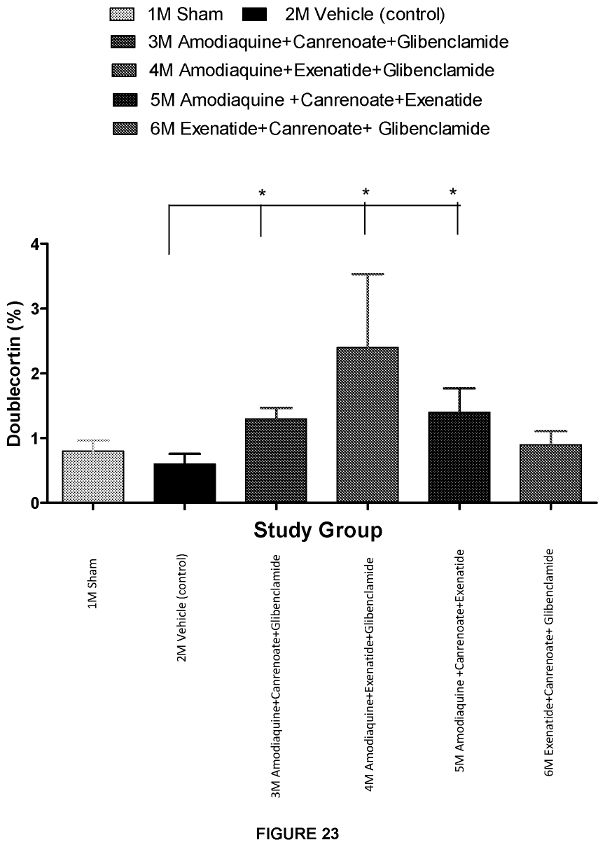

FIG. 23 shows Doublecortin staining percent of activated cells on week 4 (Averages±SEM) at 28 days for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

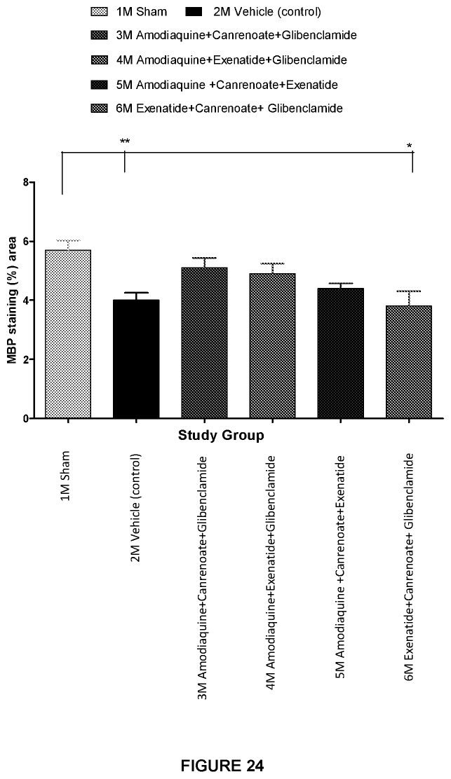

FIG. 24 shows MBP myelin damage at study termination (Averages±SEM) for selected combinations in a rat model of chronic stroke study as described in Example 3. Group 1M: sham; Group 2M: vehicle treated; Group 3M: potassium canrenoate+amodiaquine+glibenclamide; Group 4M: exenatide+amodiaquine+glibenclamide; Group 5M: exenatide+potassium canrenoate+amodiaquine; Group 6M: exenatide+potassium canrenoate+glibenclamide; Doses: amodiaquine (0.5 mg/kg); exenatide (0.05 μg/kg); potassium canrenoate (0.33 mg/kg); glibenclamide (1 μg/kg).

### EXAMPLES

The present invention is further illustrated by the following examples, which should not be construed as limiting in any way.

### Example 1: Study of Amodiaquine Combinations in a Rat Model of Vascular Dementia

### Study Objective

The purpose of the study was to evaluate the neuroprotective efficacy of amodiaquine and various combinations thereof, given intravenously 24 h after both common carotid arteries permanent ligation and then administered twice daily for three weeks, using the Wistar rat Vascular Dementia model.

### Materials

Exenatide acetate salt was obtained from Bachem AG, Switzerland. Potassium canrenoate was obtained from Pfizer, Switzerland. Glibenclamide was obtained from Tocris Bioscience. Amodiaquine was obtained from Sigma. The vehicle was saline obtained from Biological Industries.

### Study Design and Timeline

This study was set to evaluate the neuroprotective effect of various combinations administered at a low dose intravenously twice a day during three weeks in the Wistar rat Vascular Dementia model. The study (sponsored by the Applicant) was performed at Pharmaseed Ltd (Ness-Ziona, Israel) in six cycles, each one containing 12-15 rats. Test compounds were administrated 24 hours after common carotid arteries ligation twice a day for three weeks. On Day 1 both common carotid arteries were permanently ligated. Morris water maze test was performed before common carotid arteries ligation as training for baseline and on Week 4 and Week 8 thereafter. At study termination, at the end of Week 8 after Common Carotid Arteries Occlusion (CCAO) and after MWM test was completed, rats were subjected to transcardial perfusion with 2.5% buffered PFA and brains were harvested and fixed in the same solution and stored at 4° C. Histological analysis was performed according to the plan.

The experimental design and timeline for study are presented in Table 1 and Table 2 respectively.

TABLE 1
Study design

| | | | No. |
|---|---|---|---|
| | | ROA | Animals |
| | | Slow | (at |
| Group # | Treatment Group | Injection | surgery) |
| 1M | Vehicle treated controls | IV | 15* |
| 2M | Exenatide 0.05 μg/kg + | IV | 13* |
| | Potassium canrenoate 0.33 mg/kg + | | |
| | Glibenclamide 1 μg/kg | | |
| 3M | Exenatide 0.05 μg/kg + | IV | 14* |
| | Potassium canrenoate 0.33 mg/kg + | | |
| | Glibenclamide 1 μg/kg + | | |
| | Amodiaquine 20 mg/kg | | |
| 4M | Exenatide 0.05 μg/kg + | IV | 12* |
| | Potassium canrenoate 0.33 mg/kg + | | |
| | Amodiaquine 20 mg/kg | | |
| 5M | Sham operated | IV | 5 |

*An average yield of 70% was anticipated following common carotid artery ligation.
ROA = route of administration

TABLE 2
Study timeline

| | | | | Clinical | | | |
|---|---|---|---|---|---|---|---|
| Study Day/Week | | CCAO | BW | Observation | MWM | Treatment | Termination |
| On the week | | | ✓ | ✓ | ✓ | | |
| before surgery | | | | | (training) | | |
| D 1 | Surgery | ✓ | ✓ | 4 hr post-surgery, | | 24 hours after | |

| W 4 | Twice | twice a day during | ✓ | common carotid | |
|---|---|---|---|---|---|
| | a week | first two days, | | arteries ligation, | |
| | | then twice a week | | twice a day for | |
| | | | | three weeks | |
| W 8 | | | ✓ | | ✓ |

CCAO = Common Carotid Arteries Occlusion;
D = Day;
W = Week;
BW = Body Weight;
MWM = Morris Water Maze.

### Animals

Male Wistar rats were used in the study, weighing 196-320 g at study initiation.

### Animal Management

### Housing

Animal handling was performed according to guideline of the National Institute of Health (NIH) and the Association for Assessment and Accreditation of Laboratory Animal Care (AAALAC). Animals were housed in polyethylene cages (maximum 3 rats/cage) measuring 42.5×26.5×18.5 cm, with stainless steel top grill facilitating pelleted food and drinking water in plastic bottle; bedding: steam sterilized clean paddy husk (Envigo, Sani-chips cat #7090C). Bedding material was changed along with the cage at least twice a week.

### Diet

Animals were fed ad libitum by a commercial rodent diet (Teklad Certified Global 18% Protein Diet cat #: 2018SC). Animals had free access to autoclaved and acidified drinking water (pH between 2.5 and 3.5) obtained from the municipality supply.

### Environment Conditions

Animals were housed under standard laboratory conditions, air conditioned and filtered (HEPA F6/6) with adequate fresh air supply (minimum of 15 air changes/hour). Animals were kept in a climate-controlled environment. Temperature range was 18-24° C. and relative humidity range was 30-70% with 12 hours light and 12 hours dark cycle.

### Randomization

Animals were randomly allocated into cages according to Pharmaseed's SOP #027 “Random allocation of animals”.

### Procedures and Evaluations

### Surgical Procedure: Two Common Carotid Arteries Ligation

On the day of surgery anesthesia was induced on a heating pad with 4% isoflurane in a mixture of 70% N₂O and 30% O₂ and maintained with 1.5-2% isoflurane. Buprenorphine at 0.1 mg/kg was injected subcutaneously. Two common carotid artery occlusions were performed according to the method described by Hyun Joon Lee et al (Citicoline Protects Against Cognitive Impairment in a Rat Model of Chronic Cerebral Hypoperfusion, J Clin Neurol. 2009; 5(1):33-38). Both Common Carotid Arteries (CCA) were exposed through a midline neck incision and carefully dissected free from surrounding nerves and fascia. Both arteries were double ligated with a 4-0 silk suture at 8-10 mm below the visible region of the external carotid artery. The surgical wound was closed and the animals were returned to their cages to recover from anesthesia. Analgesic treatment was given again by the end of the day and twice a day during the next four days.

### Administration of Combination

Treatment started 24 hours after arteries ligation, via intravenous (IV) injection of the all mixtures except amodiaquine. Amodiaquine at 20 mg/kg was administered IP. Treatment was performed twice a day for three consecutive weeks.

### Morris Water Maze Test

The Morris water maze (MWM) test was executed to assess cognitive deficits following the Common Carotid Arteries ligation. The test was performed according to Pharmaseed's SOP 100 (Morris Water Maze Testing V6) and related publications (e.g. Brandeis R, Brandys Y and Yehuda S, “The use of the Morris Water Maze in the study of memory and learning”, Int J Neurosci. 1989; 48(1-2):29-69)

### Pre Surgery Training

Animals were trained and conditioned for one week in the Morris Water Maze, according to Pharmaseed's SOP 100 and the scientific publications (e.g. see Brandeis R et al)). Before MWM, rats' cages were transferred from the animal housing to the behavior testing room for an acclimation of about one hour. The training results on the last day were considered as baseline data for comparison. MWM test has exclusion criteria as follows: failing to escape to the platform in 90 sec. (on Day 3 of training).

### Post Common Carotid Arteries Ligation Testing

Before MWM, rats' cages were transferred from the animal housing to the behavior testing room for an acclimation of about one hour. The MWM test was performed on Week 4 and 8 after the Common Carotid Arteries ligation.

### Termination

Rats were sacrificed 8 weeks following the Common Carotid Arteries Occlusion (CCAO) and after the MWM test was completed. All rats were subjected to transcardial perfusion with 2.5% buffered PFA and brain were harvested and fixed in the same solution and stored at 4° C.

### Histological Analysis

Tissue preparation and trimming (affected hemisphere), X3 accurate cross sections of the striatum (Corpus Callosum) dorsal hippocampus and optical tract per brain. Paraffin block preparation H&E and TUNEL staining, IHC: Double Cortin for neuro-regeneration in brain sub-ventricular zone. MBP, myelin in white matter, Iba-1 for microglia and GFAP for reactive astrocytes. Olig-2 for mature Oligodendrocytes, NG2 for young Oligodendrocytes. Slides evaluation analysis; cell bodies counting at hippocampal CA1 and CA3 regions—three sections per brain, three fields per section. Morphometric analysis of neuronal death count at the hippocampal area and additional MBP, Olig-2 and NG2 morphometry was performed at the optic tract, striatum, and dorsal hippocampus areas. Pictures of the histology slides were obtained.

### Results

Morris Water Maze test

The Morris Water Maze (MWM) test was performed to evaluate vascular dementia-related cognitive impairments following the ischemic hippocampal damages, presented as learning and memory deficits. As expected, a marked decline in cognitive function was observed during testing four weeks after both CCAO in all the groups compared to the SHAM operated group 5M.

The results of the MWM test for the five study groups can be summarized as follows:
a) During the short introduction to the maze, before the surgery, the rats from the five groups performed similarly.
b) Rats that underwent only SHAM operation (5M group) performed in the cognitive test better than all the Vascular Dementia operated rats, reaching the hidden platform faster and exhibiting the effectiveness of the ischemic procedure.
c) The triple combination treated rats (2M and 4M groups) as well as the quadruple combination (3M) performed better than vehicle treated rats (1M group) during acquisition of the maze (the more sensitive learning function). This was exhibited on Week 4 mostly on the first and second trials of learning the task (marked as T1 and T2 in FIG. 1). Later on, during subsequent tests on Week 8, they showed no improvement over the Vehicle group.
d) Ischemic pronounced effect was also exhibited at the distance the animals swam to reach the platform (see FIG. 2). The various treatments presented similar pattern, with group 3M exhibiting statistical difference compared to group 1M (*p<0.05 on Week 4 Trial 2).

Histology Results: The triple combination (Group 2M) significantly reduced the apoptotic number of cells in the hippocampus as measure by Tunnel staining. Group (4M) somewhat reduced the number of apoptotic cells, though less than the Group 2M (*p<0.05). The number of pyknotic cells at the CA1 and CA3 hippocampal regions was evaluated. While CA1 cells are known to be sensitive to various toxic agents, it has been established that CA3 cells are mostly sensitive to the reduction in oxygenation and that their damage is correlated with cognitive dysfunction (T Kadar, M Silbermann, R Brandeis and A Levy, Age-related structural changes in the rat hippocampus: correlation with working memory deficiency, Brain Res 1990; 512(1):113-120; T Kadar, I Arbel, M Silbermann and A Levy, Morphological hippocampal changes during normal aging and their relation to cognitive deterioration, J Neural Transm Suppl. 1994; 44:133-143; B Shukitt-Hale, T Kadar, B E Marlowe, M J Stillman, R L Galli, A Levy, J A Devine, H R Lieberman, Morphological alterations in the hippocampus following hypobaric hypoxia, Hum Exp Toxicol. 1996; 15(4):312-319).

As shown in FIG. 3, the averages for the number of pyknotic cells in the CA1 were relatively low with no statistically significant difference between any of the treatments compared to vehicle control. The pyknotic damage in CA3, as exhibited by the vehicle treated group 1M, was massive. In this parameter the quadruple combination 3M and the triple combination 4M exhibited better significant results compared to vehicle treatment than the original 2M. No statistically significant differences were found in Iba-1, GFAP, Olig-2, NG2, Myelin density and double Cortin cells in all brain regions between all animal's groups.

### Discussion and Conclusions

Chronic cerebral hypoperfusion induced by permanent, Bilateral Common Carotid Arteries Occlusion (BCCAO) in rats, has been shown to lead to significant white matter lesions, learning and memory impairments and hippocampal neuronal damage. Thus, BCCAO in rats provided a useful model for understanding the pathophysiology of chronic cerebrovascular hypoperfusion and for screening drugs with potential therapeutic value for Vascular Dementia. Furthermore, the model is also relevant to Alzheimer's disease, as it is also accompanied by a slow developing reduction in cerebral blood flow.

The present study was carried out to determine whether the proposed treatments can ameliorate cognitive deficits induced by chronic cerebral hypoperfusion, and decrease the neuronal damage in the brain, mostly in CA-1 and CA-3 region of the hippocampus and lesions at white matter areas. The combinations were initially administrated intravenously 24 hours after the surgical procedure and then twice a day during three weeks. As expected, cognitive functional impairment was observed on week 4 after CCCA procedure. Rats that underwent only SHAM operation (5M group) performed in the cognitive test better than all the Vascular Dementia operated rats, reaching the hidden platform faster and exhibiting the effectiveness of the ischemic procedure. The triple combination treated rats (2M and 4M groups) as well as the quadruple combination (3M) performed better than vehicle treated rats (1M group) during acquisition of the maze (the more sensitive learning function). The treatment, starting 24 hours after the surgery, then given twice a day for three weeks, led to the protection against the hippocampal cell damage. The new triple combination (4M) also reduced the apoptotic cells number. Advantageously, the combinations containing amodiaquine (Groups 3M and 4M) exhibit improved activity against pyknosis and hippocampal cell death.

### Example 2: Efficacy Study of Amodiaquine Combinations in a Rat Model of Transient Middle Cerebral Artery Occlusion

### Objective

The purpose of the current study was to evaluate the neuroprotective efficacy of: a) Amodiaquine at four doses compared to Vehicle control, and b) evaluate the efficacy of Amodiaquine combinations with two other Test Items (Exenatide and Canrenoate) compared to their performance alone and compared to Vehicle control. One combination also included Glibenclamide.

Several animal models have been used to study cerebral ischemia in effort to understand its pathophysiology and to identify therapeutic strategies for minimizing the severity of ischemic damage. Focal ischemia brings about a localized brain infarction and usually induced by transient middle cerebral artery occlusion (t-MCAO) in the rat. It has gained increasing acceptance as a model for hemispheric infarction in humans. After MCAO a cortical and striatal infarct with temporal and spatial evolution occurs within the vascular region supplied by the middle cerebral artery.

### Materials

Exenatide acetate salt was obtained from Bachem AG, Switzerland. Potassium canrenoate was obtained from Pfizer, Switzerland. Glibenclamide was obtained from Tocris Bioscience. Amodiaquine was obtained from Sigma. The vehicle was saline obtained from Biological Industries.

### Animals

Male Sprague Dawley (SD) rats were used in the study, weighing 300-410 g at study initiation.

### Animal Management

### Housing

Animals' handling was performed according to guidelines of the National Institute of Health (NIH) and the Association for Assessment and Accreditation of Laboratory Animal Care (AAALAC). Animals were housed in polyethylene cages (3/cage) measuring 42.5×26.5×18.5 cm, with stainless steel top grill facilitating pelleted food and drinking water in plastic bottle; bedding: steam sterilized clean paddy husk (Envigo, Sani-chip, Cat #: 7090C) was used and bedding material was changed along with the cage at least twice a week.

### Diet

Animals were fed ad libitum a commercial rodent diet (Teklad Certified Global 18% Protein Diet, Envigo cat #2018SC). Animals had free access to standard tap drinking water obtained from the municipality supply and treated according to Pharmaseed's SOP No. 214: “Water system”. Animal feed arrived with a certificate of analysis and the water was autoclaved prior to use.

### Environment Conditions

Animals were fed ad libitum by a commercial rodent diet (Teklad Certified Global 18% Protein Diet cat #: 2018SC). Animals had free access to autoclaved and acidified drinking water (pH between 2.5 and 3.5) obtained from the municipality supply.

### Randomization

Animals were allocated into cages upon arrival according to Pharmaseed's SOP #027 “Random allocation of animals”.

### Experimental Design and Conditions

### Pain Reducing Methods

The MCAO procedure was performed under anesthesia with 4% isoflurane in a mixture of 70% N2O and 30% O2 and maintained with 1.5-2% isoflurane. Meloxicam at 2 mg/kg was administered subcutaneously (SC) before and after the surgery and once a day for the next four days. Thereafter, only if there were signs of pain and discomfort.

### Duration of the Experimental Period

Seven days following the end of acclimation. First dosing day was assigned “Day 1 and that termination was “Day 8”.

### Study Design and Timeline

The study (sponsored by the Applicant) was performed at Pharmaseed Ltd (Ness-Ziona, Israel).

Part I: This part of the study was set to evaluate the neuroprotective effect of Amodiaquine administered at four doses orally 20 minutes before reperfusion and twice a day compared to Vehicle control.

Part II: Test Items (Exenatide, Canrenoate and Glibenclamide) were evaluated in combination with Amodiaquine compared to Vehicle control. The first two were also compared to their performance alone. The study was performed in cycles, each one containing at least nine rats. Test compounds were administrated 20 minutes before reperfusion and twice a day thereafter. On Day 1, stroke was induced by the t-MCAO procedure. Neurological score (NSS), was performed before surgery, 24 hours and 7 days after t-MCAO. At study termination brains were harvested, sliced into five 2 mm thick coronal sections and stained with TTC for infarct size measurement. The experimental design and timeline for the two parts of the study are presented below.

### Study Design

### Part I: Monotherapy Study Design

Dosing regimen: 20 minutes prior to initiation of reperfusion and twice a day (every 12 hours) thereafter.

TABLE 3A

| Group | N | Treatment | Termination |
|---|---|---|---|
| 1M | 10 | Vehicle control | Day 7 after |
| 2M | 8 | Amodiaquine (20 mg/kg) | MCAO. |
| 3M | 9 | Amodiaquine (7 mg/kg) | |
| 4M | 10 | Amodiaquine (2 mg/kg) | |
| 5M | 11 | Amodiaquine (0.5 mg/kg) | |

### Part II: Combination Therapy Study Design

Dosing regimen: 20 minutes prior to initiation of reperfusion and twice a day (every 12 hours) thereafter.

TABLE 3B

| Group | N | Treatment | Termination |
|---|---|---|---|
| 6M | 9 | Amodiaquine (0.5 mg/kg) + | Day 7 after |
| | | Potassium canrenoate (0.33 mg/kg) + | MCAO. |
| | | Glibenclamide (1 μg/kg) | |
| 7M | 10 | Exenatide (0.05 μg/kg) | |
| 8M | 9 | Potassium canrenoate (0.33 mg/kg) | |
| 9M | 9 | Amodiaquine (0.5 mg/kg + | |
| | | Exenatide (0.05 μg/kg) | |
| 10M | 12 | Amodiaquine (0.5 mg/kg + | |
| | | Potassium canrenoate (0.33 mg/kg) | |
| 11M | 10 | Amodiaquine (0.5 mg/kg + | |
| | | Potassium canrenoate (0.33 mg/kg) | |
| | | Exenatide (0.05 μg/kg) | |

TABLE 4
Study timeline for both parts

| | | Clinical | | | | |
|---|---|---|---|---|---|---|
| Study Day | t-MCAO | observation | NSS | BW | Treatment | Termination |
| Before surgery | | | ✓ | ✓ | 20 minutes | |
| 1 | ✓ | ✓ | | | before reperfusion | |
| 2 | | ✓ | ✓ | ✓ | and twice a | |
| 3 | | ✓ | | ✓ | day thereafter | |
| 4 | | ✓ | | ✓ | | |
| 5 | | ✓ | | ✓ | | |
| 6 | | ✓ | | ✓ | | |
| 7 | | ✓ | ✓ | ✓ | | |
| 8 | | | | ✓ | | ✓ |

### Procedures and Evaluations

### Surgical Procedure

Transient middle cerebral artery occlusion (t-MCAO) was performed according to the method described by R. Schmid-Elsaesser et al. (Stroke; 1998; 29(10): 2162-70). The right Common Carotid artery (CCA) was exposed through a midline neck incision and carefully dissected free from surrounding nerves and fascia—from its bifurcation to the base of the skull. The Occipital artery branches of the External Carotid artery (ECA) were isolated, and these branches were dissected and coagulated. The ECA was dissected further distally and coagulated along with the terminal lingual and maxillary artery branches, just before their bifurcation. The Internal Carotid artery (ICA) was isolated and carefully separated from the adjacent Vagus nerve, and the Pterygopalatine artery was ligated close to its origin with a 5-0 nylon suture. Next a 4-0 silk suture was tied loosely around the mobilized ECA stump, and a 4 cm length of 4-0 monofilament nylon suture (the tip of the suture blunted by using a flame, and the suture was coated with polylysine, prior to insertion) was inserted through the proximal ECA into the ICA and thence into the circle of Willis, effectively occluding the MCA. The surgical wound was closed and the animals were returned to their cages to recover from anesthesia. One hour and half after occlusion rats were re-anesthetized and monofilament was withdrawn to allow reperfusion. The surgical wound was closed and rats were returned to their cages. Following surgery, animals were placed on a heating pad until their recovery from the anesthesia. All animals received subcutaneous Meloxicam at 2 mg/kg before and after surgery, daily during the next two days. They were observed frequently on the day of t-MCAO surgery and at least once daily thereafter.

### Administration of Combination

Treatment was start 20 minutes before reperfusion and then at the end of the same day, via IP administration of the test compounds. Treatment continued twice a day for six consecutive days thereafter.

The Modified Neurological Score (mNSS)

Animals were subjected to the NSS test (Schsbitz W. R., Berger C., Kollmar R., Seitz M., Tanay E., Kiessling M., Schwab S., Sommer C. Effect of brain-derived neurotrophic factor treatment and forced arm use on functional motor recovery after small cortical ischemia. Stroke. 2004; 35(4):992-997) before surgery as baseline, 24 hours and 7 days after MCAO surgery.

### Termination

On Day 8 after MCAO surgery animals were sacrificed by CO₂ exposure. Brains were collected, sliced into five 2 mm thick sections and stained with TTC and subjected to histological evaluation for infarct volume determination. After infarct volume evaluation slices was stored in PFA for future histological analysis.

### Results

### Neuroscore Evaluation During the Study

Some clinical observations that were recorded after the surgery are common following stroke induction and appeared in all groups. Neurological score (NSS) included a set of clinical-neurological tests (composite of motor, sensory, reflex and balance tests) that were used to assess the effect of Amodiaquine monotherapy and its combination with exenatide, glibenclamide and potassium canrenoate. Neuroscore was graded on a scale of 0 to 18 (in which normal score is 0 and maximal deficit score is represented by 18). Before the surgery, all animals showed normal behavior with score of zero. A sharp decline in neurological functions (increase in NSS) was recorded in all groups of rats twenty-four hours after t-MCAO. Similar and statistically significant NSS improvement was observed in Groups 2M, 3M and 5M compared to the vehicle treated (see FIG. 4). This improvement appeared already during the first test on Day 2 and continued up to Day 8. Combination of Amodiaquine with exenatide, glibenclamide and potassium canrenoate also caused significant NSS improvement in the treated groups compared to the vehicle treated control, from the first test on Day 2 up to Day 8 (see FIG. 5).

Infarct Size Infarct size was measured by image analysis using ImageJ program. As shown in FIG. 6 and FIG. 7 brain infarct size was 38% of the total hemisphere volume in the vehicle treated t-MCAO animals. In the animals treated by Amodiaquine brain infarct size decreased compared to the vehicle treated control, mostly in those treated with the higher doses. Combination of amodiaquine with exenatide, glibenclamide and potassium canrenoate also caused significant almost two-fold decrease in infarct size compared to the vehicle treated control. Less prominent decrease in infarct volume was observed in the animals treated with potassium canrenoate and amodiaquine+exenatide given 20 minutes before reperfusion.

T-tests were performed obtained for the infarct size data in order to determine statistical significance between the treatment groups. Group 6M performed significantly better than Group 10M (***p<0.001), Group 5M (**p<0.01) and Group 8M (*p<0.05). Group 11M performed significantly better than Group 10 (**p<0.01), and Groups 5M and 7M (*p<0.05). Group 10M performed significantly better than Group 8M (*p<0.05).

The performance of each group expressed as percentage of the control values is summarised in FIG. 8.

### Discussion and Conclusions

In the first part of the study, the dose-response efficacy of amodiaquine was evaluated at four doses compared to Vehicle control, using the t-MCAO stroke model in rats. In the second part, three other active agents (Exenatide, Canrenoate and Glibenclamide) were evaluated in combination with Amodiaquine compared to vehicle control, the first two also compared to their performance alone. In the first part of the study, clear differences were demonstrated between the groups treated with amodiaquine when compared to the animals in the vehicle control group, primarily at the high doses that were tested. A more pronounced effect was observed for the combined therapy.

The main observations can be summarized as follows:
None of the treatments affected the animals' body weight, well-being or mortality.
Amodiaquine monotherapy at different doses improved significantly and dose dependently the neurological score following stroke compared to the vehicle treatment.
Amodiaquine combination therapy also improved significantly the neurological score compared to the vehicle treatment.
Amodiaquine mono-therapy at different doses significantly reducing the infarct size compared to the vehicle treatment.
Amodiaquine combination therapy also reducing significantly the infarct size compared to the vehicle treatment.
The two triple combinations “Amodiaquine+Canrenoate+Glibenclamide” and
Amodiaquine+Canrenoate+Exenatide” are particularly preferred. The double combination “Amodiaquine+Exenatide” also performed well.

In view of these findings, it may be concluded that under the conditions of the present study, treatment with amodiaquine mono-therapy and amodiaquine combination therapies, when administered 20 minutes before reperfusion and twice a day after words, clearly improved sensory motor functions and decreased infarct size in the rat transient t-MCAO model.

### Example 3: Efficacy Study in the Rat Model of Transient Middle Cerebral Artery Occlusion During Continuous 28-Day Treatment

### Objective

The purpose of the current study was to evaluate the efficacy of Amodiaquine, Exenatide and Canrenoate, and their combinations, as well as one combination containing Glibenclamide to improve functional recovery following 28 days of the rat stroke induction.

### Materials

Exenatide acetate salt was obtained from Bachem AG, Switzerland. Potassium canrenoate was obtained from Pfizer, Switzerland. Glibenclamide was obtained from Tocris Bioscience. Amodiaquine was obtained from Sigma. The vehicle was saline obtained from Biological Industries.

### Animals

Male Sprague Dawley (SD) rats were used in the study, weighing 295-330 g at study initiation.

### Animal Management

### Housing

Animals' handling was performed according to guidelines of the National Institute of Health (NIH) and the Association for Assessment and Accreditation of Laboratory Animal Care (AAALAC). Animals were housed in polyethylene cages (3/cage) measuring 42.5×26.5×18.5 cm, with stainless steel top grill facilitating pelleted food and drinking water in plastic bottle; bedding: steam sterilized clean paddy husk (Envigo, Sani-chip, Cat #: 7090C) was used and bedding material was changed along with the cage at least twice a week.

### Diet

Animals were fed ad libitum a commercial rodent diet (Teklad Certified Global 18% Protein Diet, Envigo cat #2018SC). Animals had free access to standard tap drinking water obtained from the municipality supply and treated according to Pharmaseed's SOP No. 214: “Water system”. Animal feed arrived with a certificate of analysis and the water was autoclaved prior to use.

### Environment Conditions

Animals were fed ad libitum by a commercial rodent diet (Teklad Certified Global 18% Protein Diet cat #: 2018SC). Animals had free access to autoclaved and acidified drinking water (pH between 2.5 and 3.5) obtained from the municipality supply.

### Randomization

Animals were allocated into cages upon arrival according to Pharmaseed's SOP #027 “Random allocation of animals”.

### Experimental Design and Conditions

### Pain Reducing Methods

The MCAO procedure was performed under anesthesia with 4% isoflurane in a mixture of 70% N2O and 30% O2 and maintained with 1.5-2% isoflurane. Meloxicam at 2 mg/kg was administered subcutaneously (SC) before and after the surgery and once a day for the next four days. Buprenorphine at 0.01 mg/kg was administered after the surgery and at the end of the working day. Thereafter, only if there were signs of pain and discomfort.

### Duration of the Experimental Period

First dosing day was assigned “Day 1 and termination was twenty-eight days following surgery on “Day 29”.

### Study Design and Timeline

Test Items—Amodiaquine, Canrenoate, Glibenclamide and Exenatide were evaluated in various combinations, compared to Vehicle control treatment or SHAM operated rats. The study was performed in cycles, each one containing ten rats. Test compounds were administrated immediately after reperfusion and twice a day thereafter. On Day 1, stroke was induced by t-MCAO procedure. Neurological score (NSS), was performed before surgery for baseline, 24 hours after t-MCAO and every week thereafter. Animals with score 10 and above were included in the study. Baseline evaluation was performed for Stepping Test, Forelimb Placement Test and then on Weeks 2 and 4 of the study. Elevated Plus Maze Test was performed on Weeks 2 and 4 of the study and Object Recognition Test on Week 4. At study termination, animals were anesthetized and submitted to trans-cardiac perfusion with 0.9% saline followed by 2.5% buffered PFA. Brains were harvested and fixed with the same fixative and stored at 4° C. Brain samples were embedded in paraffin, sectioned and stained with H&E and with other staining for histological markers detailed previously. The experimental design and timeline are presented below:

Study design: The study (sponsored by the Applicant) was performed at Pharmaseed Ltd (Ness-Ziona, Israel).

| | | | Dosing | Route of | |
|---|---|---|---|---|---|
| Group | N | Treatment* | Regimen | Admin | Termination |
| 1M | 6 | SHAM operated | NA | NA | Day 29 after |
| 2M | 12* | Vehicle control | Immediately | IP | t-MCAO. ~7-8 |
| | | | after reperfusion | | animals/group after |
| 3M | 12* | Amodiaquine (0.5 mg/kg) + | and twice daily | | exclusion/inclusion |
| | | Canrenoate (0.33 mg/kg) + | (Every 12 hours) | | criteria |
| | | Glibenclamide (1 μg/kg) | thereafter for | | |
| 4M | 12* | Amodiaquine (0.5 mg/kg) + | 28 days | | |
| | | Exenatide (0.05 μg/kg) + | | | |
| | | Glibenclamide (1 μg/kg) | | | |
| 5M | 12* | Amodiaquine (0.5 mg/kg) + | | | |
| | | Canrenoate (0.33 mg/kg) + | | | |
| | | Exenatide (0.05 μg/kg) | | | |
| 6M | 12* | Exenatide (0.05 μg/kg) + | | | |
| | | Canrenoate (0.33 mg/kg) + | | | |
| | | Glibenclamide (1 μg/kg) | | | |

*An average yield of around 80% was anticipated following MCAO surgery resulting in 7-9 animals/group at the end of the study.

### Study Timeline:

| Study | | | | Stepping | | | | | |
|---|---|---|---|---|---|---|---|---|---|
| Day/Week | | t-MCAO | NSS | Test | FPT | EPMT | ORT | Treatment | Termination |
| Before | | | ✓ | ✓ | ✓ | | | Immediately | |
| surgery | | | | | | | | after reperfusion | |
| Day | 1 | ✓ | ✓ | | | | | and twice daily | |
| Week | 1 | | ✓ | | | | | (Every 12 hours) | |
| Week | 2 | | ✓ | ✓ | ✓ | ✓ | | thereafter | |
| Week | 3 | | ✓ | | | | | | |
| Week | 4 | | ✓ | ✓ | ✓ | ✓ | ✓ | | |
| Day | 29 | | | | | | | | ✓ |

NSS = Neurological Score;
FPT = Forelimb Placement Test;
EPMT = Elevated Plus Maze Test;
ORT = Object Recognition Test

### Procedures and Evaluations

### Surgical Procedure-t-MCA

Transient middle cerebral artery occlusion (t-MCAO) was performed according to the method described by R. Schmid-Elsaesser et al. The right Common Carotid artery (CCA) was exposed through a midline neck incision and carefully dissected free from surrounding nerves and fascia—from its bifurcation to the base of the skull. The Occipital artery branches of the External Carotid artery (ECA) were isolated, and these branches were dissected and coagulated. The ECA was dissected further distally and coagulated along with the terminal lingual and maxillary artery branches, just before their bifurcation. The Internal Carotid artery (ICA) was isolated and carefully separated from the adjacent Vagus nerve, and the Pterygopalatine artery was ligated close to its origin with a 5-0 nylon suture. Next a 4-0 silk suture was tied loosely around the mobilized ECA stump, and a 4 cm length of 4-0 monofilament nylon suture (the tip of the suture blunted by using a flame, and the suture was coated with polylysine, prior to insertion) was inserted through the proximal ECA into the ICA and thence into the circle of Willis, effectively occluding the MCA. The surgical wound was closed and the animals were returned to their cages to recover from anesthesia. One hour and half after occlusion rats were re-anesthetized and monofilament was withdrawn to allow reperfusion. The surgical wound was closed and rats were returned to their cages. Following surgery, animals were placed on a heating pad until their recovery from the anesthesia. All animals received subcutaneous Meloxicam at 2 mg/kg before and after surgery, daily during the next two days. They were observed frequently on the day of t-MCAO surgery and at least once daily thereafter.

### Test Items Administration

Treatment was start immediately after reperfusion and then at the end of the same day, via IP administration of the test compounds at dose volume of 1 mL/kg. Treatment continued twice a day for four consecutive weeks thereafter.

Animals were subjected to the NSS test before surgery as baseline, 24 hours and every week thereafter for four weeks.

### Forelimb Placement Test

Forelimb Placement Test (FPT) was performed before MCAO surgery (for baseline), on Week 2 and on Week 4. The animals were held closely to a tabletop and scores of the rat's abilities to place the forelimbs on the table surface in response to whisker, visual, tactile, or proprioceptive stimulation was recorded. Separate sub-scores were obtained for each mode of sensory input and added to give total scores. The Forelimb Placement Test scores range from 0=normal to 12=maximally impaired. Typically, there is a slow and steady recovery of the scores during the first month after stroke.

### Stepping Test

Animals were tested for forelimb akinesia in a Stepping Test (ST), according to Pharmaseed's SOP #111 (“Rats Stepping Test”). ST was performed before MCAO surgery, on Week 2 and on Week 4. The animal was held with its hind limbs fixed in one hand and the forelimb, not to be monitored, in the other, while the unrestrained fore-paw touches the table. The number of adjusting steps was counted while the animal was moved sideways along the table surface (85 cm during approximately five seconds), in the forehand & backhand direction for both forelimbs.

### Elevated Plus Maze Test (Pharmaseed SOP #120)

Animals were tested for the levels of anxiety in the Elevated Plus Maze Test on Week 2 and on Week 4. The normal behavior for rats in the elevated plus maze includes exploratory activity and they spend equal time in open and closed arms of the maze. Monitoring the behavior in this task (i.e., visits in the open versus closed arms) reflects the conflict between the rodent's preference for protected areas (e.g., closed arms) and their innate motivation to explore novel environments.

### Object Recognition Test (Pharmaseed SOP #116)

Animals were tested on Week 4 in the novel Object Recognition task for cognitive memory testing. Rats were introduced to a new object compared to a familiar object. The number of visits and time spent near either novel or familiar objects was recorded. Under normal conditions, novel objects are visited more frequently and for longer periods of time than familiar objects.

### Termination

On Day 29 after MCAO surgery animals were anesthetized and submitted to transcardiac perfusion with 0.9% saline followed by 2.5% buffered PFA. Brains were harvested, fixed with the same fixative and stored at 4° C.
Macroscopic examination of the brain
Histological analyses included:
   Tissue preparation and trimming (affected hemisphere) for all surviving animals, X8 accurate cross sections of the stroke lesion with an interval of 100 microns. Arears in interest: α-penumbra, Striatum and Dorsal hippocampus.
   Paraffin block preparation.
   H&E staining, infarct volume was analyzed for all surviving animals.
   The following analysis was performed in all the three areas of interest.
   TUNEL—method for apoptosis (n=46).
   IHC: Double Cortin for neuro-regeneration in brain ventricles and NeuN (for mature neurons) (n=46).
   Myeline analysis: IHC for MBP, Olig-2 and NG2, were analyzed using digital image analysis (n=46).
   Inflammation analysis: IHC for Iba-1 and GFAP, were analyzed using digital image analysis (n=46).
   Morphometric analysis of neuro death count, MBP, Olig-2, NG2, GFAP, and Iba-2 (n=46).

### Results

### Neuro-Score Evaluation During the Study

Neurological score (NSS) included a set of clinical-neurological tests (composite of motor, sensory, reflex and balance tests) that were used to assess the effect of Amodiaquine in combination with exenatide, glibenclamide and potassium canrenoate. Neuroscore was graded on a scale of 0 to 18 (in which normal score is 0 and maximal deficit score is represented by 18). Before the surgery, all animals showed normal behavior with score of zero. As shown in FIG. 9, in all groups of rats that had t-MCAO surgery, a sharp decline in neurological functions (increase in NSS) was observed twenty-four hours after t-MCAO with some improvement over time thereafter. For group 6M improvement became statistically significant only starting on Week 2.

Statistically significant differences, according to two-way ANOVA followed by Bonferroni post-hoc comparisons, were found between Group 2M (Vehicle control) and the drug treated groups: 3M Amodiaquine (0.5 mg/kg)+Canrenoate (0.33 mg/kg)+Glibenclamide (1 μg/kg), 4M Amodiaquine (0.5 mg/kg)+Exenatide (0.05 μg/kg)+Glibenclamide (1 μg/kg), 5M Amodiaquine (0.5 mg/kg)+Canrenoate (0.33 mg/kg)+Exenatide (0.05 μg/kg) on Day 2 and on Weeks 1-4. For group 6M—Exenatide (0.05 μg/kg)+Canrenoate (0.33 mg/kg)+Glibenclamide (1 μg/kg)—statistically significant difference started to show only from Week 2 onward. The figure shows averages±SEM; ** indicates p<0.01, *** indicates p<0.001.

### Stepping Test (ST)

Animals were tested for forelimb akinesia in the Stepping Test, commonly used for measurement of neuromuscular function, as an index for motoric function of the animals. On the day before surgery all animals behaved normally and similar to the SHAM operated group. As shown in FIG. 10, all t-MCAO groups exhibited statistically significant impaired performance (lower scores) compared to the SHAM operated group 1M. Statistically significant improvement in motor function over time (higher scores) was observed in all the treated animal groups compared to the Vehicle treated group 2M (at p<0.001).

### Forelimb Placement Test (FPT)

Forelimb Placement Test was used to assess somatosensory and sensory motor deficits. On the day before surgery, all animals behaved normally. Functional improvement was observed all the treated groups compared to the Vehicle treated group, which was statistically significant at p<0.001 (according to two-way ANOVA followed by Bonferroni post-hoc comparisons). The results are presented in FIG. 11.

### Elevated Plus Maze Test (Pharmaseed SOP #120)

Animals were tested for the levels of anxiety in the Elevated Plus Maze Test on Week 2 and on Week 4. As shown in FIG. 13 all animals spent more time in closed arm of the maze. Statistically significant difference was found on Week 2 and 4 between Vehicle treated group 2M and group 3M, treated with Amodiaquine+Canrenoate+Glibenclamide. The figure shows averages±SEM; *** indicates p<0.001 according to two-way ANOVA followed by Bonferroni post-hoc comparisons. No statistically significant differences were found between all other groups during the study.

### Object Recognition Test (Pharmaseed SOP #116)

Animals were tested on Week 4 in the Object Recognition Test for cognitive memory testing. Rats were introduced to a new object compared to a familiar object. The time spent near either novel or familiar objects was recorded. Vehicle treated animals exhibited impaired function compared to SHAM operated rats (at p<0.05), spending more time exploring the familiar object. Statistically significant improvement compared to Vehicle treatment was found for group 3M (treated with the combination of Amodiaquine+Canrenoate+Glibenclamide), and for group 4M (treated by the combination of Amodiaquine+Exenatide+Glibenclamide). Test results are presented in FIG. 12. The figure shows averages±SEM; * indicates p<0.05; ** indicates p<0.01, using two-way ANOVA followed by Bonferroni post-hoc comparisons.

### Infarct Size

Infarct size was measured via image analysis using ImageJ program. As shown in FIG. 14 below, treatment in all the groups containing Amodiaquine—3M, 4M and 5M (but not 6M)—exhibited statistically significant decreased brain infarct size (more than two folds smaller), compared to the Vehicle treated control. The two groups 3M and 5M showed statistical improvement over group 6M.

### Histology Results

### Apoptosis as Measured Via TUNEL

The important specific parameter for neuroprotection—apoptosis as measured via TUNEL staining, revealed that all t-MCAO groups presented significantly higher number of apoptotic cells compared to the SHAM operated group. The triple combination (Group 3M) significantly reduced the apoptotic number of cells compared to the Vehicle treated control (Group 2M) (*p<0.05). The combination without Amodiaquine (Group 6M) at this stage exhibited higher percentage of apoptotic cells than Group 3M treatment (**p<0.01). See FIG. 17.

### Myelin Basic Protein (MBP) Density Measured by IHC

MBP density represented the damage to myelin. Control Vehicle treated animals had significant decrease in myelin density compared to SHAM operated animals. Group 6M treated without Amodiaquine had also significant decrease in myelin density compared to SHAM operated animals. However, all combination groups that included Amodiaquine were not significantly different from the SHAM operated animals suggesting a protective effect of these combinations (see FIG. 24).

### Olig-2 Mean Number of Cells Measured by Morphometric Analysis

The triple combination group 4M significantly increased Oligo-2 density compared to triple combination group 3M (Group 1M), (*p<0.05) according to one-way ANOVA. No statistically significant differences were found between SHAM operated group and all other groups (FIG. 18).

### NG2 Oligodendrocyte Progenitor Cells

NG2 oligodendrocyte progenitor cells increased statistically significant in Vehicle treated group (Group 2M) compared to SHAM operated group (1M) and were also high in two of the treated combinations (4M and 5M) (P<0.05 according to t-test). The triple combination groups (3M and 6M) at this stage significantly reduced the activated microglia area compared to Vehicle control group (Group 2M), (*p<0.05; **p<0.01) according to one-way ANOVA. For further details see FIG. 19.

### NeuN Neurons

The Vehicle control group (Group 2M) significantly reduced the NeuN density compared to SHAM control (Group 1M), (*p<0.05). The triple combination group (3M) at this stage also reduced significantly the NeuN density compared to SHAM operated control group (Group 1M), (*p<0.05) according to one-way ANOVA. For details see FIG. 20. Three other parameters that were evaluated via morphometric analysis at the end of Week 4 were GFAP, 11a-1 and Doublecortin, in the brain regions as depicted in FIG. 21, FIG. 22 and FIG. 23.

### GFAP

Morphometric analysis for Glial Fibrillary Acidic Protein (GFAP), representing reactive astrocytes, exhibited statistically significant GFAP increase in the Vehicle treated control group 2M compared to SHAM operated group 1M (at p<0.001) and in 6M (at p<0.01). The triple combinations treated groups 3M, 4M and 5M reduced GFAP markers compared to the vehicle treated group 2M (at p<0.001, p<0.001 and p<0.05 respectively), according to one-way ANOVA. See FIG. 21.

Iba-1 staining

The results of the morphometric analysis for Iba-1 staining (representing microglia cellsin the ischemic brain) exhibited the same trend as shown for GFAP, namely the highest value for group 2M (Vehicle treated) and the lowest value for triple combination groups (3M, 4M and 5M and SHAM operated group 1M), (**p<0.01; ***p<0.001) according to one-way ANOVA. Group 6M was statistically significant (at p<0.001) only compared to the SHAM operated 1M group. See FIG. 22.

### Doublecortin Staining

The results of the morphometric analysis for Doublecortin staining (representing the neurogenesis process) exhibited higher values for the triple combinations groups 3M, 4M and 5M compared to the group 2M (Vehicle treated), (at p<0.05) according to t-test. See FIG. 23.

### Conclusions

The results of Example 3 are consistent with previous stroke model studies (placebo group, infarct size, NSS test). Progressive improvement of NSS is observed over time (Day 28 vs Day 8). Furthermore, relatively better improvement in infarct size with triple combinations is observed vs control at 28 days compared to the results at 8 days (from 12% to 8% and from 15% to 10%, vs from 38% to 27%). Significant improvement in memory tests is also observed.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

Abdallah D M, et al. Glibenclamide ameliorates ischemia-reperfusion injury via modulating oxidative stress and inflammatory mediators in the rat hippocampus. Brain Res. 2011; 1385: 257-62.
Atri A. The Alzheimer's Disease Clinical Spectrum: Diagnosis and Management. Med Clin North Am. 2019; 103: 263-293.
Bai J, Lyden P D. Revisiting cerebral post-ischemic reperfusion injury: new insights in understanding reperfusion failure, hemorrhage, and edema. Int J Stroke. 2015; 10: 143-52.
Brandeis R, Brandys Y and Yehuda S, “The use of the Morris Water Maze in the study of memory and learning”, Int J Neurosci. 1989; 48(1-2):29-69.
Berge S M, et al. Pharmaceutical salts. J Pharm Sci. 1977; 66: 1-19.
Beygui F, et al. High plasma aldosterone levels on admission are associated with death in patients presenting with acute ST-elevation myocardial infarction. Circulation. 2006; 114: 2604-10.
Bovelli D, et al. Cardiotoxicity of chemotherapeutic agents and radiotherapy-related heart disease: ESMO Clinical Practice Guidelines. Ann Oncol. 2010; 21 (Suppl 5): v277-82.
Bullock B P, et al. Tissue distribution of messenger ribonucleic acid encoding the rat glucagon-like peptide-1 receptor. Endocrinology. 1996; 137: 2968-78.
Caffes N, et al. Glibenclamide for the treatment of ischemic and hemorrhagic stroke. Int J Mol Sci. 2015; 16: 4973-84.
Chaudhuri K R, et al. Unmet needs in Parkinson's disease: New horizons in a changing landscape. Parkinsonism Relat Disord. 2016; 33 (Suppl 1): S2-S8.
Chen et al. Role of neuroinflammation in neurodegenerative diseases (Review), Molecular Medicine Reports 13: 3391-3396, 2016.
Chu Y et al. (2002) Age-related decreases in Nurr1 immunoreactivity in the human substantia nigra. J Comp Neurol 450(3):203-214.
Chu Y, et al. (2006) Nurr1 in Parkinson's disease and related disorders. J Comp Neurol 494(3):495-514).
Cordiner R L M, Pearson E R. Reflections on the sulfonylurea story: A drug class at risk of extinction or a drug class worth reviving? Diabetes Obes Metab. 2019; 21: 761-771
Dae M W, et al. Effect of endovascular cooling on myocardial temperature, infarct size, and cardiac output in human-sized pigs. Am J Physiol Heart Circ Physiol. 2002; 282: H1584-91.
Dinh Q N, et al. Cell-specific mineralocorticoid receptors: future therapeutic targets for stroke? Neural Regen Res. 2016; 11(8):1230-1.
Ebert S E, Jensen P, Ozenne B, Armand S, Svarer C, Stenbaek D S et al. Molecular imaging of neuroinflammation in patients after mild traumatic brain injury: a longitudinal 123 I-CLINDE SPECT study. Eur J Neurol 2019. doi:10.1111/ene.13971.
Garber A J, et al. CONSENSUS STATEMENT BY THE AMERICAN ASSOCIATION OF CLINICAL ENDOCRINOLOGISTS AND AMERICAN COLLEGE OF ENDOCRINOLOGY ON THE COMPREHENSIVE TYPE 2 DIABETES MANAGEMENT ALGORITHM—2019 EXECUTIVE SUMMARY. Endocr Pract. 2019; 25: 69-100.
Garden G A, October 2013, “Epigenetics and the modulation of neuroinflammation”. Neurotherapeutics. 10 (4): 782-8.
Gendelman H E, December 2002, “Neural immunity: Friend or foe?”. Journal of Neurovirology. 8 (6): 474-9.
Glass C K, et al. (2010) Mechanisms underlying inflammation in neurodegeneration. Cell 140(6):918-934).
Göke R, et al. Exendin-4 is a high potency agonist and truncated exendin-(9-39)-amide an antagonist at the glucagon-like peptide 1-(7-36)-amide receptor of insulin-secreting beta-cells. J. Biol. Chem. 268: 19650-19655.
Götberg M, et al. Mild hypothermia reduces acute mortality and improves hemodynamic outcome in a cardiogenic shock pig model. Resuscitation. 2010; 81: 1190-6.
Gouzoulis-Mayfrank E, Daumann J. Neurotoxicity of drugs of abuse—the case of methylenedioxyamphetamines (MDMA, ecstasy), and amphetamines. Dialogues Clin Neurosci. 2009; 11: 305-17.
Gribble F M, Reimann F. Sulfonylurea action revisited: the post-cloning era. Diabetologia. 2003; 46: 875-891.
Hambrock A, et al. Resveratrol binds to the sulfonylurea receptor (SUR) and induces apoptosis in a SUR subtype-specific manner. J Biol Chem. 2007; 282: 3347-56.
Hemphill J C 3rd, et al. Guidelines for the management of spontaneous intracerebral hemorrhage: a guideline for healthcare professionals from the American Heart Association/American Stroke Association. Stroke. 2015; 46: 2032-2060.
Hochman J S, et al. Early revascularization in acute myocardial infarction complicated by cardiogenic shock. SHOCK Investigators. Should We Emergently Revascularize Occluded Coronaries for Cardiogenic Shock. N Engl J Med. 1999; 341: 625-34.
Holmes D R Jr, et al. Contemporary reperfusion therapy for cardiogenic shock: the GUSTO-1 trial experience. The GUSTO-1 Investigators. Global Utilization of Streptokinase and Tissue Plasminogen Activator for Occluded Coronary Arteries. J Am Coll Cardiol. 1995; 26: 668-74.
Huang K, et al. Exploratory Analysis of Oral Glibenclamide in Acute Ischemic Stroke. Acta Neurol Scand. 2019 May 29; doi: 10.1111/ane.13134.
Inzucchi S E, et al. Management of hyperglycemia in type 2 diabetes, 2015: a patient-centered approach: update to a position statement of the American Diabetes Association and the European Association for the Study of Diabetes. Diabetes Care. 2015; 38: 140-9.
Jeon et al. Aging Dis., 2020; 11(3):705-724); Jiang C, et al. (2005) Age-dependent dopaminergic dysfunction in Nurr1 knockout mice. Exp Neurol 191(1):154-162.
Jiang B, et al. Role of Glibenclamide in Brain Injury After Intracerebral Hemorrhage. Transl Stroke Res. 2017; 8: 183-193.
Jorm A F, Korten A E, Henderson A S (November 1987). “The prevalence of dementia: a quantitative integration of the literature”. Acta Psychiatrica Scandinavica. 76 (5): 465-79. doi:10.1111/j.1600-0447.1987.tb02906.x. PMID 3324647.
Kern K B. Usefulness of cardiac arrest centers—extending lifesaving post-resuscitation therapies: the Arizona experience. Circ J. 2015; 79(6): 1156-63.
Kim J Y, et al. Multilayer nanoparticles for sustained delivery of exenatide to treat type 2 diabetes mellitus. Biomaterials. 2013; 34: 8444-9.
Kim et al. PNAS; Jul. 14, 2015; vol. 112; no. 28; 8756-8761; Nuclear receptor Nurr1 agonists enhance its dual functions and improve behavioral deficits in an animal model of Parkinson's disease.
Kim et al. Stem Cell Research 17 (2016) 534-543; The pharmacological stimulation of Nurr1 improves cognitive functions via enhancement of adult hippocampal neurogenesis.
King Z A, et al. Profile of intravenous glyburide for the prevention of cerebral edema following large hemispheric infarction: evidence to date. Drug Des Devel Ther. 2018; 12: 2539-2552.
Kinoshita et al, Journal of Neuroimmunology 330 (2019) 48-54, A Nurr1 agonist amodiaquine attenuates inflammatory events and neurological deficits in a mouse model of intracerebral haemorrhage.
Kronick S L, et al. Part 4: Systems of Care and Continuous Quality Improvement: 2015 American Heart Association Guidelines Update for Cardiopulmonary Resuscitation and Emergency Cardiovascular Care. Circulation. 2015; 132 (18 Suppl 2): S397-413.
Kunte H, et al. Hemorrhagic transformation of ischemic stroke in diabetics on sulfonylureas. Ann Neurol. 2012; 72: 799-806.
Kurson et al. The Journal of Headache and Pain (2021) 22:55, Migraine and neuroinflammation: the inflammasome perspective.
Le W D, et al. 2003 Mutations in NR4A2 associated with familial Parkinson disease. Nat Genet 33(1):85-89.
Hyun Joon Lee et al (Citicoline Protects Against Cognitive Impairment in a Rat Model of Chronic Cerebral Hypoperfusion, J Clin Neurol. 2009; 5(1):33-38.
Liddelow et al, Nature, 2017, January 26; 541(7638): 481-487.
Lopez M S, et al. Resveratrol neuroprotection in stroke and traumatic CNS injury. Neurochem Int. 2015; 89: 75-82.
Mayer C L, Huber B R, Peskind E (October 2013). “Traumatic brain injury, neuroinflammation, and post-traumatic headaches”. Headache. 53 (9): 1523-30.
Mehta R I et al. Sur1-Trpm4 Cation Channel Expression in Human Cerebral Infarcts. J Neuropathol Exp Neurol. 2015; 74: 835-49.
Nishida H et al. Glimepiride treatment upon reperfusion limits infarct size via the phosphatidylinositol 3-kinase/Akt pathway in rabbit hearts. J Pharmacol Sci. 2009; 109: 251-6.
Nyström T, et al. Effects of glucagon-like peptide-1 on endothelial function in type 2 diabetes patients with stable coronary artery disease. Am J Physiol Endocrinol Metab. 2004; 287: E1209-15.
Okuda M. A multidisciplinary overview of cardiogenic shock. Shock. 2006; 25: 557-70. Oluwatosin et al. 2010, Acta Poloniae Pharmaceutica n Drug Research, Vol. 67 No. 5 pp. 529n536, 2010, Vasorelaxation induced by amodiaquine in rat superior mesenteric arteries: In Vivo And in Vitro Studies.
Onyango et al., Biomedicines 2021, 9, 524. Neuroinflammation in Alzheimer's Disease Patel A D, et al. Glibenclamide reduces hippocampal injury and preserves rapid spatial learning in a model of traumatic brain injury. J Neuropathol Exp Neurol. 2010; 69: 1177-90.
Patel R A G, et al. Neuroprotection in the Treatment of Acute Ischemic Stroke. Prog Cardiovasc Dis. 2017; 59: 542-548.
Patel R D, Saver J L. Evolution of reperfusion therapies for acute brain and acute myocardial ischemia: a systematic, comparative analysis. Stroke. 2013; 44: 94-8.
Powers W J, et al. 2018 Guidelines for the early management of patients with acute ischemic stroke: a guideline for healthcare professionals from the American Heart Association/American Stroke Association. Stroke. 2018; 49: e46-e99; updated 2019; 50: e344-e418.
Proks P, et al. Sulfonylurea stimulation of insulin secretion. Diabetes. 2002; 51 (Suppl 3): S368-76.
Sacchetti P, et al (2006); “Multiple signaling pathways regulate the transcriptional activity of the orphan nuclear receptor NURR1”. Nucleic Acids Research. 34 (19): 5515-27. doi:10.1093/nar/gkl712. PMC 1636490. PMID 17020917
Saijo K, Winner B, Carson C T, Collier J G, Boyer L, Rosenfeld M G, Gage F H, Glass C K (April 2009). “A Nurr1/CoREST pathway in microglia and astrocytes protects dopaminergic neurons from inflammation-induced death”. Cell. 137 (1): 47-59. doi:10.1016/j.cell.2009.01.038. PMC 2754279. PMID 19345186.
Savitz S I, et al. Reconsidering Neuroprotection in the Reperfusion Era. Stroke. 2017; 48: 3413-3419.
Schmid-Elsaesser R, et al. A critical reevaluation of the intraluminal thread model of focal cerebral ischemia: evidence of inadvertent premature reperfusion and subarachnoid hemorrhage in rats by laser-Doppler flowmetry. Stroke. 1998; 29: 2162-70.
Simard J M, et al. Newly expressed SUR1-regulated NC(Ca-ATP) channel mediates cerebral edema after ischemic stroke. Nat Med. 2006; 12: 433-40.
Simard, J M, et al. Glibenclamide reduces inflammation, vasogenic edema, and caspase-3 activation after subarachnoid hemorrhage. Journal of Cerebral Blood Flow and Metabolism. 2009; 29; 317-330.
Simard J M, et al. Glibenclamide-10-h Treatment Window in a Clinically Relevant Model of Stroke. Transl Stroke Res. 2012; 3: 286-95.
Simard, J M, et al. Does inhibiting Sur1 complement rt-PA in cerebral ischemia? Ann. N. Y. Acad. Sci. 2012, 1268, 95-107.
Sola D, et al. Sulfonylureas and their use in clinical practice. Arch Med Sci. 2015; 11: 840-8.
Streit W J, Mrak R E, Griffin W S (July 2004). “Microglia and neuroinflammation: a pathological perspective”. Journal of Neuroinflammation. 1 (1): 14.
Struthers A D. Aldosterone: cardiovascular assault. Am Heart J. 2002; 144 (5 Suppl): S2-7.
Tan F, et al. Protective effect of treatment with low-dose gliclazide in a model of middle cerebral artery occlusion and reperfusion in rats. Brain Res. 2014; 1560: 83-90.
Tosun C, et al. Inhibition of the Sur1-Trpm4 channel reduces neuroinflammation and cognitive impairment in subarachnoid hemorrhage. Stroke. 2013; 44: 3522-8.
Valsecchi et al. Dis. Model Mech. 2020; 13(5):dmm043513; Wilkinson C M, et al. Glibenclamide, a Sur1-Trpm4 antagonist, does not improve outcome after collagenase-induced intracerebral hemorrhage. PLoS One. 2019; 14: e0215952.
Woo S K, et al. The sulfonylurea receptor 1 (Sur1)-transient receptor potential melastatin 4 (Trpm4) channel. J Biol Chem. 2013; 288: 3655-67.
Xu Z M, et al. Glibenclamide Attenuates Blood-Brain Barrier Disruption in Adult Mice after Traumatic Brain Injury. J Neurotrauma. 2017; 34: 925-933.
Xu F, et al. Glibenclamide ameliorates the disrupted blood-brain barrier in experimental intracerebral hemorrhage by inhibiting the activation of NLRP3 inflammasome. Brain Behav. 2019; 9: e01254.
Zannad F, Radauceanu A. Effect of MR blockade on collagen formation and cardiovascular disease with a specific emphasis on heart failure. Heart Fail Rev. 2005; 10: 71-8.
Zhang G, et al. A Protective Role of Glibenclamide in Inflammation-Associated Injury. Mediators Inflamm. 2017; 2017: 3578702.
Zhu S, et al. Glibenclamide Enhances the Therapeutic Benefits of Early Hypothermia after Severe Stroke in Rats. Aging Dis. 2018; 9: 685-695.

1. A combination comprising:
(a) a first component which is a Nurr1 agonist; and
(b) at least one additional component selected from:
(i) an aldosterone antagonist;
(ii) an insulin modulator; and
(iii) a sulfonylurea.

2. A combination according to claim 1 wherein the Nurr1 agonist is selected from amodiaquine, chloroquine, hydroxychloroquine and glafenine, and pharmaceutically acceptable salts thereof, and is more preferably amodiaquine, or a pharmaceutically acceptable salt thereof.

3. A combination according to claim 1 or claim 2 comprising:
amodiaquine, or a pharmaceutically acceptable salt thereof;
an aldosterone antagonist; and
at least one of the following components: an insulin modulator, and a sulfonylurea.

4. A combination according to any one of claims 1 to 3 wherein the aldosterone antagonist is potassium canrenoate, or a structural or functional analogue thereof.

5. A combination according to any one of claims 1 to 4 which comprises amodiaquine, or a pharmaceutically acceptable salt thereof; an aldosterone antagonist; and an insulin modulator.

6. A combination according to claim 5 wherein the insulin modulator is selected from exenatide, and structural and functional analogues thereof, and pharmaceutically acceptable salts thereof.

7. A combination according to claim 6 wherein the exenatide structural or functional analogue is selected from lixisenatide, albiglutide, semaglutide, liraglutide, taspoglutide and dulaglutide (LY2189265).

8. A combination according to any one of claims 1 to 4 which comprises amodiaquine, or a pharmaceutically acceptable salt thereof; an aldosterone antagonist; and a sulfonylurea.

9. A combination according to claim 8 wherein the sulfonylurea is selected from glibenclamide, and pharmaceutically acceptable salts thereof, and structural and functional analogues thereof.

10. A combination according to claim 9 wherein the glibenclamide structural or functional analogue is selected from acylhydrazone, sulfonamide and sulfonylthiourea derivatives of glibenclamide, glimepiride, glipizide and gliclazide, preferably gliclazide.

11. A combination according to claim 1 which comprises:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

12. A combination according to claim 1 which comprises:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

13. A combination according to claim 1 which comprises:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof.

14. A combination according to claim 1 which comprises:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof; and
glibenclamide, or a structural or functional analogue thereof, or a pharmaceutically acceptable salt thereof.

15. A combination according to claim 1 comprising:
amodiaquine, or a pharmaceutically acceptable salt thereof; and
exenatide, or a structural or functional analogue thereof, or a pharmaceutically acceptable salt thereof.

16. A combination according to any one of claims 2 to 15 which comprises a hydrochloride salt of amodiaquine.

17. A combination according to any preceding claim which comprises at least one further active pharmaceutical ingredient (API) selected from a beta blocker, a renin-angiotensin inhibitor, a statin (HMG-CoA reductase inhibitor), an inhibitor of platelet activation or aggregation, a phosphodiesterase-3 inhibitor, a calcium sensitizer, an antioxidant, and an anti-inflammatory agent.

18. A pharmaceutical composition comprising a combination according to any preceding claim and a pharmaceutically acceptable carrier, diluent or excipient.

19. A pharmaceutical product comprising:
(a) a first component which is a Nurr1 agonist; and
(b) at least one additional component selected from:
(i) an aldosterone antagonist;
(ii) an insulin modulator; and
(iii) a sulfonylurea

20. A pharmaceutical product according to claim 19 comprising:
amodiaquine, or a pharmaceutically acceptable salt thereof;
an aldosterone antagonist; and
at least one of the following components: an insulin modulator, and a sulfonylurea.

21. A pharmaceutical product according to claim 20 comprising:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

22. A pharmaceutical product according to claim 20 comprising:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

23. A pharmaceutical product according to claim 20 comprising:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof.

24. A pharmaceutical product according to claim 20 comprising:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof; and
glibenclamide, or a structural or functional analogue thereof, or a pharmaceutically acceptable salt thereof.

25. A pharmaceutical product according to claim 20 comprising:
amodiaquine, or a pharmaceutically acceptable salt thereof; and
exenatide, or a structural or functional analogue thereof, or a pharmaceutically acceptable salt thereof.

26. A combination according to any one of claims 1 to 17, or a pharmaceutical composition according to claim 18, or a pharmaceutical product according to any one of claims 19 to 25, for use in the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for use in providing cardioprotection against cardiotoxic drugs, or for use in providing neuroprotection.

27. A combination according to any one of claims 1 to 17, or a pharmaceutical composition according to claim 18, or a pharmaceutical product according to any one of claims 19 to 25, for use according to claim 26, wherein the ischemia and/or reperfusion injury is ischemia and/or reperfusion injury of the brain, heart, lung, kidney, preferably cerebral ischemia, cerebral reperfusion injury or stroke.

28. A combination according to any one of claims 1 to 17, or a pharmaceutical composition according to claim 18, or a pharmaceutical product according to any one of claims 19 to 25, for use according to claim 26, for use in treating or preventing ischemic stroke.

29. A combination according to any one of claims 1 to 17, or a pharmaceutical composition according to claim 18, or a pharmaceutical product according to any one of claims 19 to 25, for use according to claim 26, for use in treating or preventing neuroinflammation or a neuroinflammatory disorder.

30. A combination according to any one of claims 1 to 17, or a pharmaceutical composition according to claim 18, or a pharmaceutical product according to any one of claims 19 to 25, for use according to claim 29, wherein the neuroinflammatory disorder is selected from Alzheimer's disease, Parkinson's disease, multiple sclerosis, Acute disseminated encephalomyelitis (ADEM), Accute Optic Neuritis (AON), Transverse Myelitis and Neuromyelitis Optica (NMO).

31. A combination according to any one of claims 1 to 17, or a pharmaceutical composition according to claim 18, or a pharmaceutical product according to any one of claims 19 to 25, for use according to claim 29, wherein the neuroinflammation is associated with traumatic brain injury, spinal cord injury, aging, schizophrenia, depression, migraine, epilepsy, neuropathic pain, Down Syndrome, autism, preterm infant, glaucoma or a viral infection.

32. A combination according to any one of claims 1 to 17, or a pharmaceutical composition according to claim 18, or a pharmaceutical product according to any one of claims 19 to 25, for use according to claim 26, for use in treating or preventing a neurodegenerative disease, preferably selected from Parkinson's disease, Alzheimer's disease, Amyotrophic lateral sclerosis, Huntington's disease, amyotrophic lateral sclerosis (ALS) and vascular dementia.

33. A combination according to any one of claims 1 to 17, or a pharmaceutical composition according to claim 18, or a pharmaceutical product according to any one of claims 19 to 25, for use according to claim 26, for providing neuroprotection.

34. A combination according to any one of claims 1 to 17, or a pharmaceutical composition according to claim 18, or a pharmaceutical product according to any one of claims 19 to 25, which is formulated for intravenous administration.

35. A combination according to any one of claims 1 to 17, or a pharmaceutical composition according to claim 18, or a pharmaceutical product according to any one of claims 19 to 25, which is formulated for subcutaneous administration.

36. A combination according to any one of claims 1 to 17, or a pharmaceutical composition according to claim 18, or a pharmaceutical product according to any one of claims 19 to 25, which is formulated for oral administration.

37. A combination according to any one of claims 1 to 17, or a pharmaceutical composition according to claim 18, or a pharmaceutical product according to any one of claims 19 to 25, which is formulated for intranasal administration.

38. A method of treating and/or preventing one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection, said method comprising simultaneously, sequentially or separately administering to a subject in need thereof:
(a) a first component which is a Nurr1 agonist; and
(b) at least one additional component selected from:
(i) an aldosterone antagonist;
(ii) an insulin modulator, and
(iii) a sulfonylurea.

39. A method according to claim 38 which comprises simultaneously, sequentially or separately administering to a subject in need thereof:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

40. A method according to claim 38 which comprises simultaneously, sequentially or separately administering to a subject in need thereof:
amodiaquine, or a pharmaceutically acceptable salt thereof;
potassium canrenoate, or a structural or functional analogue thereof; and
glibenclamide, or a structural or functional analogue thereof or a pharmaceutically acceptable salt thereof.

41. A method according to claim 38 which comprises simultaneously, sequentially or separately administering to a subject in need thereof:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

42. A method according to claim 38 which comprises simultaneously, sequentially or separately administering to a subject in need thereof:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof; and
glibenclamide, or a structural or functional analogue thereof, or a pharmaceutically acceptable salt thereof.

43. A method according to claim 38 which comprises simultaneously, sequentially or separately administering to a subject in need thereof:
amodiaquine, or a pharmaceutically acceptable salt thereof; and
exenatide, or a structural or functional analogue thereof, or a pharmaceutically acceptable salt thereof.

44. A method according to any one claims 38 to 43 wherein the ischemia and/or reperfusion injury is ischemia and/or reperfusion injury of the brain, heart, lung, kidney, preferably cerebral ischemia, cerebral reperfusion injury or stroke.

45. A method according to any one claims 38 to 44 for treating or preventing ischemic stroke.

46. A method according to any one claims 38 to 43 for treating or preventing a neurodegenerative disease, preferably selected from Parkinson's disease, Alzheimer's disease, Huntington's disease, amyotrophic lateral sclerosis and vascular dementia.

47. A method according to any one claims 38 to 43 for providing neuroprotection.

48. A method according to any one claims 38 to 43 for treating or preventing neuroinflammation or a neuroinflammatory disorder.

49. A method according to any one of claims 38 to 48 wherein the components are administered parenterally, preferably intravenously.

50. A method according to any one of claims 38 to 48 wherein the components are administered orally.

51. A method according to any one of claims 38 to 48 wherein the components are administered subcutaneously.

52. A method according to any one of claims 38 to 48 wherein the components are administered intranasally.

53. A method according to any one of claims 38 to 48 wherein the components are administered in a first administration phase and at least one second administration phase, wherein the components in said first administration phase are administered intravenously, and the components in said at least one second administration phase are administered orally.

54. A method according to any one of claims 38 to 48 wherein the components are administered in a first administration phase and at least one second administration phase, wherein the components in said first administration phase are administered intravenously, and the components in said at least one second administration phase are administered subcutaneously.

55. A method according to any one of claims 40 to 42 which comprises administering glibenclamide at a dosage of about 0.001 to about 30 μg/kg body weight of the subject, more preferably about 0.01 to about 5 μg/kg.

56. A method according to any one of claims 39, 41, 42 or 43 which comprises administering exenatide, or pharmaceutically acceptable salt thereof, at a dosage of about 0.001 to about 1.5 μg/kg body weight of the subject, preferably about 0.01 to about 0.1 μg/kg.

57. A method according to any one of claims 39, 40 or 42 which comprises administering potassium canrenoate at a dosage of about 0.03 to about 10 mg/kg body weight of the subject, preferably about 0.3 to about 5 mg/kg.

58. A method according to any one of claims 39 to 43 which comprises administering amodiaquine, or pharmaceutically acceptable salt thereof, at a dosage of about 0.01 to about 20 mg/kg body weight of the subject, preferably about 0.1 to about 10 mg/kg, more preferably about 0.1 to about 1 mg/kg, even more preferably about 0.5 or 0.1 mg/kg.

59. A method according to any one of claims 38 to 58 which comprises simultaneously administering the components to said subject.

60. Use of:
(a) a first component which is a Nurr1 agonist; and
(b) at least one additional component selected from:
(i) an aldosterone antagonist;
(ii) an insulin modulator, and
(iii) a sulfonylurea;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

61. Use according to claim 60 which comprises the use of:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

62. Use according to claim 60 which comprises the use of:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

63. Use according to claim 60 which comprises the use of:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

64. Use according to claim 60 which comprises the use of:
amodiaquine, or a pharmaceutically acceptable salt thereof; and
exenatide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

65. Use according to claim 60 which comprises the use of:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof; and
glibenclamide, or a structural or functional analogue thereof, or a pharmaceutically acceptable salt thereof;
in the manufacture of a medicament for the treatment and/or prevention of one or more of ischemia and/or reperfusion injury, neuroinflammation, a neuroinflammatory disorder, stroke, a neurodegenerative disease, neonatal asphyxia, cardiac arrest, cardiogenic shock and acute myocardial infarction, or for providing cardioprotection against cardiotoxic drugs, or for providing neuroprotection.

66. Use of a combination comprising:
(a) a first component which is a Nurr1 agonist; and
(b) at least one additional component selected from:
(i) an aldosterone antagonist;
(ii) an insulin modulator, and
(iii) a sulfonylurea;
for treating and/or preventing ischemia and/or reperfusion injury in an ex vivo organ prior to or during transplantation.

67. Use according to claim 66 of a combination comprising:
amodiaquine, or a pharmaceutically acceptable salt thereof;
potassium canrenoate, or a structural or functional analogue thereof; and
exenatide, or a structural or functional analogue thereof, or a pharmaceutically acceptable salt thereof.

68. Use according to claim 66 of a combination comprising:
amodiaquine, or a pharmaceutically acceptable salt thereof;
exenatide, or a structural or functional analogue thereof, or a pharmaceutically acceptable salt thereof; and
glibenclamide, or a structural or functional analogue thereof, or a pharmaceutically acceptable salt thereof.

69. Use according to claim 66 of a combination comprising:
amodiaquine, or a pharmaceutically acceptable salt thereof;
potassium canrenoate, or a structural or functional analogue thereof; and
glibenclamide, or a structural or functional analogue thereof, or a pharmaceutically acceptable salt thereof.

70. Use according to claim 66 of a combination comprising:
amodiaquine, or a pharmaceutically acceptable salt thereof;
potassium canrenoate, or a structural or functional analogue thereof;
exenatide, or a structural or functional analogue thereof, or a pharmaceutically acceptable salt thereof; and
glibenclamide, or a structural or functional analogue thereof, or a pharmaceutically acceptable salt thereof.

71. Use according to claim 66 of a combination comprising:
amodiaquine, or a pharmaceutically acceptable salt thereof; and
exenatide, or a structural or functional analogue thereof, or a pharmaceutically acceptable salt thereof;
for treating and/or preventing ischemia and/or reperfusion injury in an ex vivo organ prior to or during transplantation.
